(19) United States
(12) Patent Application Publication
(10) Pub. No.: US 2022/0386875 A1
(43) Pub. Date: Dec. 8, 2022

(54) MEASURING APPARATUS AND BIOLOGICAL INFORMATION MEASURING APPARATUS

(71) Applicants: Yoshihiro OBA, Miyagi (JP); Ryosuke KASAHARA, Kanagawa (JP); Yoshio WADA, Miyagi (JP); Toshihide SASAKI, Miyagi (JP)

(72) Inventors: Yoshihiro OBA, Miyagi (JP); Ryosuke KASAHARA, Kanagawa (JP); Yoshio WADA, Miyagi (JP); Toshihide SASAKI, Miyagi (JP)

(21) Appl. No.: 17/771,026

(22) PCT Filed: Oct. 23, 2020
(86) PCT No.: PCT/JP2020/039987

§ 371 Date: Apr. 22, 2022

(30) Foreign Application Priority Data
Oct. 28, 2019 (JP) 2019-195633
Oct. 28, 2019 (JP) 2019-195636
Nov. 6, 2019 (JP) 2019-201307
Nov. 6, 2019 (JP) 2019-201786

(51) Int. Cl. A61B 5/00

(52) U.S. Cl. CPC A61B 5/0075; A61B 5/682; A61B 2562/0238; A61B 2562/16

(57) ABSTRACT

A measuring apparatus (100a, 1a) includes a light source (110) configured to emit probe light; a total reflection member (16) in contact with a to-be-measured object and configured to cause total reflection of the probe light that is incident; a light intensity detector (17) configured to detect light intensity of the probe light exiting from the total reflection member (16); an output unit (2) configured to output a measurement value obtained on the basis of the light intensity; a first support (31) supporting the light source (110) and the light intensity detector (17); and a second support (32) provided to the first support (31), detachable from the first support (31), and supporting the total reflection member (16).

### TECHNICAL FIELD

The present application relates to a measuring apparatus and a biological information measuring apparatus.

### BACKGROUND ART

In recent years, the number of patients with diabetes has increased worldwide, and noninvasive blood glucose level measurement without requiring blood sampling is desired. A variety of methods have been proposed for measuring biological information such as a blood glucose level using light, such as near-infrared, mid-infrared, or Raman spectroscopy. In particular, the mid-infrared region is the fingerprint region where glucose absorption is high, and the sensitivity of the measurement can be increased in comparison to the near-infrared region.

A light emitting device such as a quantum cascade laser (QCL) is available as a light source in the mid-infrared region, but the number of necessary light sources corresponds to the number of wavelengths used. From the viewpoint of miniaturization of the apparatus, it is desirable to reduce the number of wavelengths of the mid-infrared region to a few wavelengths.

A method using glucose absorption peak wavelengths (1035 cm⁻¹, 1080 cm⁻¹, and 1110 cm⁻¹) has been proposed (see, for example, PTL 1) in order to accurately measure glucose concentration using an attenuated total reflection (ATR) method at a specific wavelength region such as the mid-infrared region.

In such a measuring apparatus, when an optical part, such as a total reflection member contained in the apparatus, is in contact with a lip or the like of a to-be-measured person, it may be undesirable to use the same measuring apparatus for a different person in terms of safety and hygiene. In addition, measurement accuracy may deteriorate if dust or residue adheres to the measuring apparatus or if the measuring apparatus is scratched. Therefore, it is desirable to enable a part of the measuring apparatus to be detached, and then, maintained, i.e., cleaned, replaced with a new part, or the like.

A measuring apparatus in which a part of the measuring apparatus can be detachably mounted is disclosed (see, for example, PTL 2). In the measuring apparatus, a light source, such as a light emitting device, an optical part, such as a light waveguide, and a photodetector, such as a light receiver, are formed on a substrate and replaceable.

### SUMMARY OF INVENTION

### Technical Problem

However, in the measuring apparatus of PTL 2, the cost of the measuring apparatus may become higher as a result of the light source, the optical part, and the photodetector being replaced together.

An object of the present invention is to provide a measuring apparatus that ensures safety while reducing the cost of the measuring apparatus.

### Solution to Problem

A measuring apparatus according to one aspect of the present invention includes a light source configured to emit probe light; a total reflection member configured to, in contact with a to-be-measured object, cause total reflection of the probe light that is incident; a light intensity detector configured to detect light intensity of the probe light exiting from the total reflection member; an output unit configured to output a measurement value obtained on the basis of the light intensity; a first support supporting the light source and the light intensity detector; and a second support detachably provided to the first support and supporting the total reflection member.

### Effects of Invention

According to the aspect of the present invention, it is possible to provide a measuring apparatus that ensures safety while reducing the cost of the measuring apparatus Other objects, features and advantages of the present invention will become more apparent from the following detailed description when read in conjunction with the accompanying drawings.

### BRIEF DESCRIPTION OF DRAWINGS

FIG. 1 is a diagram illustrating an overall configuration of a blood glucose level measuring apparatus according to a first embodiment.

FIG. 2 depicts a function of an ATR prism.

FIG. 3 is a perspective view depicting a structure of the ATR prism.

FIG. 4 is a perspective view depicting a structure of a hollow fiber.

FIG. 5 is a block diagram of an exemplary hardware configuration of a processing unit according to the first embodiment.

FIG. 6 is a block diagram illustrating an example of a functional configuration of a processing unit according to the first embodiment.

FIG. 7A is a diagram illustrating a case in which first probe light is used in an example of a probe light switching operation.

FIG. 7B is a diagram illustrating a case in which second probe light is used in the example of the probe light switching operation.

FIG. 7C is a diagram illustrating a case in which third probe light is used in the example of the probe light switching operation.

FIG. 8 is a flowchart illustrating an example of an operation of the blood glucose level measuring apparatus according to the first embodiment.

FIG. 9A depicts probe light intensity in a comparative example.

FIG. 9B depicts probe light intensity changed in three or more levels.

FIG. 10A depicts a cross-sectional light intensity distribution of probe light, with respect to probe light positional shift correction.

FIG. 10B depicts a cross-sectional light intensity distribution of probe light having a positional shift, with respect to probe light positional shift correction.

FIG. 10C depicts a cross-sectional light intensity distribution of probe light with a speckle, with respect to probe light positional shift correction.

FIG. 10D depicts a cross-sectional light intensity distribution of probe light with a speckle having a positional shift, with respect to probe light positional shift correction.

FIG. 11A depicts a function of an incidence face of the ATR prism with respect to total reflection of probe light in a case of a smooth incidence face.

FIG. 11B depicts a function of an incidence face of the ATR prism with respect to total reflection of probe light in a case of a diffusing incidence face.

FIG. 11C depicts the diffusing incidence face.

FIG. 11D depicts a hollow incidence face.

FIG. 11E depicts a protruding incidence face.

FIG. 12A depicts a positioning error between first and second hollow optical fibers and the ATR prism, where the ATR prism is not in contact with a living body.

FIG. 12B depicts a positioning error between the first and second hollow optical fibers and the ATR prism, where a living body is in contact with a first total reflection face of the ATR prism.

FIG. 12C depicts a positioning error between the first and second hollow optical fibers and the ATR prism, where a living body is in contact with a second total reflection face of the ATR prism.

FIG. 13 depicts supports of the first and second hollow optical fibers and the ATR prism.

FIG. 14A depicts a comparative example of a light source driving current.

FIG. 14B depicts an example of a high-frequency-modulated light source driving current.

FIG. 15A illustrates a top view of an example of a configuration of a blood glucose level measuring apparatus according to a second embodiment.

FIG. 15B illustrates a front view of the example of the configuration of the blood glucose level measuring apparatus according to the second embodiment.

FIG. 15C illustrates a side view of the example of the configuration of the blood glucose level measuring apparatus according to the second embodiment.

FIG. 16A illustrates a front view of an example of a configuration of a blood glucose level measuring apparatus according to a third embodiment.

FIG. 16B illustrates a side view of the example of the configuration of the blood glucose level measuring apparatus according to the third embodiment.

FIG. 16C illustrates a detailed view of a part A of FIG. 16A.

FIG. 17A illustrates a first variant of the part A of FIG. 16A.

FIG. 17B illustrates a second variant of the part A of FIG. 16A.

FIG. 17C illustrates a third variant of the part A of FIG. 16A.

FIG. 18A illustrates a front view of a variant of a light guide.

FIG. 18B illustrates a side view of the variant of the light guide.

FIG. 19A illustrates a front view of another variant of the light guide.

FIG. 19B illustrates a side view of the other variant of the light guide.

FIG. 20A illustrates a front view of an example of a configuration of a blood glucose level measuring apparatus according to a fourth embodiment.

FIG. 20B illustrates a B-B cross-sectional view of FIG. 20A.

FIG. 21A is a view illustrating a structure of an optical member provided in a blood glucose level measuring apparatus in a comparative example.

FIG. 21B is a view illustrating a structure of an optical member provided in a blood glucose level measuring apparatus according to a fifth embodiment.

FIG. 22 is an enlarged view illustrating a slope surface depicted in FIG. 21B.

FIG. 23 is a view illustrating a structure of an optical member according to a first variant of the fifth embodiment.

FIG. 24 is a view illustrating a structure of an optical member according to a second variant of the fifth embodiment.

FIG. 25 is a view illustrating a structure of an optical member according to a third variant of the fifth embodiment.

FIG. 26A is a diagram illustrating an example of a manufacturing process of the optical member, in particular, depicting a structure of the optical member.

FIG. 26B is a diagram illustrating the example of the manufacturing process of the optical member, in particular, depicting the optical member during the manufacturing process.

FIG. 26C is a diagram illustrating the example of the manufacturing process of the optical member, in particular, depicting the optical member during the manufacturing process.

FIG. 26D is a diagram illustrating the example of the manufacturing process of the optical member, in particular, depicting the optical member during the manufacturing process.

FIG. 26E is a diagram illustrating the example of the manufacturing process of the optical member, in particular, depicting the optical member during the manufacturing process.

FIG. 27 depicts an example of incident probe light at a Brewster angle.

FIG. 28 is a timing chart depicting an example of switching timing of probe light, (a) depicting a state of a first shutter, (b) depicting a state of a second shutter, (c) depicting a state of a third shutter, and (d) depicting an output signal of a photodetector.

FIG. 29 is a flowchart illustrating an example of an operation of a blood glucose level measuring apparatus according to a seventh embodiment.

FIG. 30 is a diagram depicting an example of a method of visually recognizing a contact between an ATR prism and a lip.

FIG. 31 depicts a diagram illustrating an overall configuration of a blood glucose level measuring apparatus according to an eighth embodiment.

FIG. 32 is an enlarged view illustrating a contact position between a piezoelectric drive unit and a first hollow optical fiber.

FIG. 33A depicts a function of the piezoelectric drive unit, in particular, a probe light image according to a comparative example.

FIG. 33B is a view of an A-A cross-sectional light intensity distribution of FIG. 33A.

FIG. 33C is a probe light image according to the eighth embodiment.

FIG. 33D is a view of a B-B cross-sectional light intensity distribution of FIG. 33C.

FIG. 34 is a diagram illustrating an overall configuration example of a blood glucose level measuring apparatus according to a first variant.

FIG. 35 is a view illustrating an example of driving of a lens.

FIG. 36 is a diagram illustrating an overall configuration example of a blood glucose level measuring apparatus according to a second variant.

FIG. 37A depicts a mirror driving example in which the mirror is vibrated by the piezoelectric drive unit.

FIG. 37B depicts another example where the mirror is vibrated by a motor.

FIG. 37C depicts yet another example where the mirror is oscillated by a MEMS mirror.

FIG. 38A is a view illustrating an ATR prism according to a ninth embodiment, where measurement sensitivity areas are at both first and second total reflection faces.

FIG. 38B depicts another example where only one measurement sensitivity area is at the center of the second total reflection face.

FIG. 38C depicts yet another example where a plurality of measurement sensitivity areas are provided at the second total reflection face.

FIG. 39A is a diagram illustrating an example of a configuration of a pressure detector according to a tenth embodiment, where the single pressure detector is provided.

FIG. 39B depicts another example where two pressure detectors are provided at both ends of the ATR prism.

FIG. 39C depicts yet another example where a plurality of pressure detectors are provided.

FIG. 40A depicts a state of the ATR prism according to the tenth embodiment with respect to a lip of a living body, and, in particular, a state before the ATR prism comes into contact with the lip.

FIG. 40B depicts a state where the living body puts the ATR prism in the mouth.

FIG. 41 is a block diagram illustrating an example of a functional configuration of a processing unit according to the tenth embodiment.

FIG. 42 is a diagram depicting relationships between a contact pressure of the ATR prism to a lip and absorbance.

FIG. 43A depicts an example arrangement of a pressure sensor with respect to a support, where the single pressure sensor is provided.

FIG. 43B depicts another example where the pressure sensor is provided at one end of the ATR prism.

FIG. 43C depicts yet another example where a plurality of pressure sensors are provided.

FIG. 44 is a diagram illustrating an example of positional relationships between a pressure sensor, a support, and an ATR prism in a thickness direction.

FIG. 45A depicts another example of positional relationships between the pressure sensor, support, and ATR prism in the thickness direction, where the pressure sensor is placed on a second total reflection face.

FIG. 45B depicts yet another example where the pressure sensors are placed on both sides of the first total reflection face and the second total reflection face.

FIG. 46 is a block diagram illustrating an example of a functional configuration of a processing unit according to an eleventh embodiment.

FIG. 47 is a diagram illustrating an example of a temperature detection result and a result of obtaining blood glucose level data before correction.

FIG. 48 is a diagram depicting correlations between a sublingual temperature and a blood glucose level.

FIG. 49 is a diagram illustrating an example of a temperature detection result and a corrected blood glucose level data obtaining result.

FIG. 50 is a block diagram illustrating an example of a functional configuration of a processing unit according to a twelfth embodiment.

FIG. 51 is a diagram depicting correlations of reference absorbance with second absorbance and third absorbance.

FIG. 52 is a diagram depicting absorbance at a single absorbance measurement.

FIG. 53 is a diagram depicting correlations of reference absorbance with second absorbance and third absorbance at a single absorbance measurement.

### DESCRIPTION OF EMBODIMENTS

Hereinafter, the embodiments of the present invention will be described with reference to the drawings. In each drawing, the same elements are provided with the same reference numerals, and duplicate descriptions may be omitted.

<Description of Terms of First Embodiment>

(Mid-Infrared Region)

A mid-infrared region refers to a wavelength region of the range between 2 and 14 μm, which is an example of a specific wavelength region.

(Probe Light)

Probe light refers to light used for absorbance measurement and biological information measurement. In the first embodiment, total reflection of probe light occurs on a total reflection member, the probe light is attenuated by a living body, and then the probe light is detected by a light intensity detector.

(ATR Method)

An attenuated total reflection (ATR) method is a method of obtaining an absorption spectrum of a to-be-measured object by using a penetrating field (evanescent waves) generated, from a total reflection face of a total reflection member such as an ATR prism in contact with the to-be-measured object, upon total reflection from the total reflection member.

(Absorbance)

Absorbance is a dimensionless amount that indicates the degree of reduction in light intensity when light passes through an object. In the first embodiment, an attenuation caused as a result of a penetrating field generated from a total reflection face into a living body is measured as absorbance by the ATR method.

(Blood Glucose Level)

A blood glucose level refers to the concentration of glucose (glucose) in blood.

(Detection Value)

In the first embodiment, a detection value refers to a value detected by a light intensity detector.

(Wavenumber)

The relationship between a wavelength λ(μm) and a wavenumber k(cm⁻¹) is “k=10000/λ”.

Hereinafter, the first embodiment will be described with reference to examples of a blood glucose level measuring apparatus (an example of a biological information measuring apparatus) for measuring a blood glucose level (an example of biological information) on the basis of absorbance measured using the ATR prism (an example of a total reflection member).

### First Embodiment

First, a blood glucose level measuring apparatus 100 according to a first embodiment will now be described.

In the first embodiment, a plurality of probe lights having different wavelengths in the mid-infrared region are used to irradiate a total reflection member provided in contact with a living body, and absorbance with respect to each of the plurality of probe lights is obtained on the basis of the ATR method, and a blood glucose level is obtained on the basis of the absorbance obtained.

<Overall Configuration Example of Blood Glucose Level Measuring Apparatus 100>

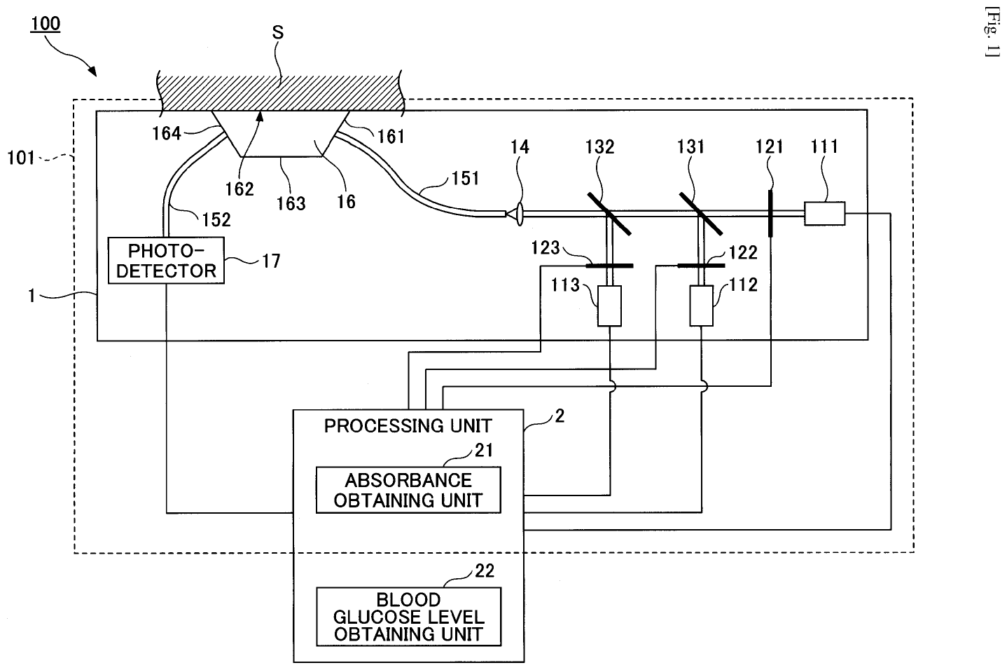

FIG. 1 is a diagram illustrating an example of the overall configuration of the blood glucose level measuring apparatus 100. As depicted in FIG. 1, the blood glucose level measuring apparatus 100 includes a measuring unit 1 and a processing unit 2.

The measuring unit 1 is an optical head for implementing the ATR method and outputs a detection signal of probe light attenuated by a living body to the processing unit 2. The processing unit 2 obtains absorbance data on the basis of the detection signal, obtains a blood glucose level on the basis of the absorbance data, and outputs the blood glucose level.

The measuring unit 1 includes a first light source 111, a second light source 112, a third light source 113, a first shutter 121, a second shutter 122, and a third shutter 123. The measuring unit 1 further includes a first half mirror 131, a second half mirror 132, a coupling lens 14, a first hollow optical fiber 151, an ATR prism 16, a second hollow optical fiber 152, and a photodetector 17.

The processing unit 2 includes an absorbance obtaining unit 21 and a blood glucose level obtaining unit 22. An absorbance measuring apparatus 101 includes the measuring unit 1 and the absorbance obtaining unit 21 as being enclosed by a broken line in FIG. 1.

The first light source 111, the second light source 112, and the third light source 113 in the measuring unit 1 are respectively quantum cascade lasers electrically connected to the processing unit 2 and each emitting laser light in the mid-infrared region in response to a control signal from the processing unit 2.

In the first embodiment, the first light source 111 emits laser light having a wavenumber of 1050 cm⁻¹ as first probe light, the second light source 112 emits laser light having a wavenumber of 1070 cm⁻¹ as second probe light, and the third light source 113 emits laser light having a wavenumber of 1100 cm⁻¹ as third probe light.

These types of laser light with wavenumbers of 1050 cm⁻¹, 1070 cm⁻¹, and 1100 cm⁻¹ correspond to the wavenumbers of absorption peaks of glucose, respectively, and the absorbances can be measured using these wavenumbers to accurately measure glucose concentrations on the basis of absorbances.

The first shutter 121, the second shutter 122, and the third shutter 123 are electromagnetic shutters electrically connected to the processing unit 2, respectively, and each controlled to open/close in accordance with a control signal from the processing unit 2.

When the first shutter 121 is opened, the first probe light from the first light source 111 passes through the first shutter 121 to the first half mirror 131. On the other hand, when the first shutter 121 is closed, the first probe light is blocked by the first shutter 121 and does not reach the first half mirror 131.

When the second shutter 122 is opened, the second probe light from the second light source 112 passes through the second shutter 122 to the first half mirror 131. On the other hand, when the second shutter 122 is closed, the second probe light is blocked by the second shutter 122 and does not reach the first half mirror 131.

Similarly, when the third shutter 123 is opened, the third probe light from the third light source 113 passes through the third shutter 123 to the second half mirror 132. On the other hand, when the third shutter 123 is closed, the third probe light is blocked by the third shutter 123 and does not reach the second half mirror 132.

The first half mirror 131 and the second half mirror 132 are optical elements for transmitting a portion of the incident light and reflecting the rest. Such an optical element can be obtained by placing an optical thin film which transmits a portion of the incident light and reflects the rest on a substrate that is transparent to the incident light.

However, each of these half mirrors is not limited to a half mirror using an optical thin film, and may be obtained by forming a diffractive structure by which a portion of the incident light is transmitted and the rest is reflected (diffracted) on a substrate that is transparent to the incident light. The use of such a diffractive structure is suitable for reducing light absorption.

The first half mirror 131 transmits first probe light that has passed through the first shutter 121 and reflects second probe light that has passed through the second shutter 122. The second half mirror 132 transmits first probe light and second probe light, respectively, and reflects third probe light that has passed through the third shutter 123.

It is desirable that light intensity ratio between transmitted light and reflected light in each of the first and second half mirrors 131 and 132 be approximately 1:1, but the light intensity ratio may be adjusted according to probe light intensity emitted by each light source or the like.

Any one of first through third probe lights having passed through the first half mirror 131 or the second half mirror 132 is guided to the first hollow optical fiber 151 via the coupling lens 14 and propagates in the first hollow optical fiber 151 to be guided to the ATR prism 16 via an incidence face 161 of the ATR prism 16.

The ATR prism 16 is an optical prism that propagates, while causing total reflection of, any one of first through third probe lights incident on the incidence face 161 and exiting from the outgoing face 164. As depicted in FIG. 1, a first total reflection face 162 of the ATR prism 16 is in contact with a living body S (an example of a to-be-measured object).

First through third probe lights guided to the ATR prism 16 repeat undergoing total reflection by each of the first total reflection face 162 and a second total reflection face 163 opposite the first total reflection face 162 and is guided to the second hollow optical fiber 152 through the outgoing face 164.

The first through third probe lights guided by the second hollow optical fiber 152 reach the photodetector 17. The photodetector 17 is a detector capable of detecting light of a wavelength in the mid-infrared region. The photodetector 17 converts any one of received first through third probe lights into an electrical signal corresponding to the light intensity and outputs an electrical signal to the processing unit 2 as a detection signal. The photodetector 17 is a photo diode (PD) for infrared rays, a mercury cadmium telluride (MCT) detection element, a bolometer, or the like. The photodetector 17 is an example of a light intensity detector. Hereinafter, when the first through third probe lights are not distinguished, the term “probe light” may be used to simply refer to as any one of the first through third probe lights.

The processing unit 2 is an information processing apparatus such as a personal computer (PC). The absorbance obtaining unit 21 of the processing unit 2 obtains absorbance data with respect to each probe light on the basis of a detection signal of the photodetector 17 and outputs the obtained absorbance data to the blood glucose level obtaining unit 22. The blood glucose level obtaining unit 22 obtains blood glucose level data (blood glucose level information) of a living body on the basis of the absorbance data with respect to each probe light.

In FIG. 1, the measuring unit 1 is enclosed by a solid line and the absorbance measuring apparatus 101 is enclosed by the broken line in order for easily understanding the configuration of the measuring unit 1 and the elements included in the absorbance measuring apparatus 101. However, these lines do not represent housings or the like. The ATR prism 16 is not provided in a housing and can come into contact with any portion of a living body with at least one of the first total reflection face 162 or the second total reflection face 163.

### <Function and Configuration of ATR Prism 16>

Next, the function of the ATR prism 16 will be described with reference to FIG. 2. As depicted in FIG. 2, the ATR prism 16 of the measuring unit 1 is in contact with a living body S. Each probe light incident on the ATR prism 16 is attenuated correspondingly to an infrared absorption spectrum a particular living body S has. The attenuated probe light is received by the photodetector 17 and the light intensity is detected for each probe light. The detection signals are input to the processing unit 2, and the processing unit 2 obtains and outputs absorbance data and blood glucose level data on the basis of the detection signals.

The ATR method is useful for spectroscopic detection with respect to the mid-infrared region where absorption intensity of glucose is obtained. An infrared ATR method utilizes a high refractive index ATR prism 16 to be irradiated with probe light, which is infrared light, and “penetration” of a field occurs when total reflection occurs at the interface between the ATR prism 16 and an external environment (e.g., a living body S). As a result of measurement being performed with the ATR prism 16 in contact with a living body S to be measured, the penetrating field is absorbed by the living body S.

As a result of using infrared light of a wide wavelength range from 2 μm through 12 μm as probe light, light of a wavelength generated due to molecular vibrational energy of a living body S is absorbed, and the light absorption appears in a form of a dip at the corresponding wavelength of the probe light transmitted through the ATR prism 16. This technology is particularly advantageous for infrared spectroscopy using weak power probe light because a large amount of detected light can be caused to pass through the ATR prism 16.

When infrared light is used, the depth of light penetrating from the ATR prism 16 to a living body S is only a few microns, and the light does not reach a capillary that is several hundred microns deep. However, it is known that blood plasma and another ingredient penetrate into a skin or a mucosal cell as a tissue fluid (interstitial fluid). A blood glucose level can be measured by detecting a glucose ingredient present in the tissue fluid.

The concentration of a glucose ingredient in a tissue fluid is thought to increase as the glucose ingredient approaches a capillary, and the ATR prism is constantly pressed at a constant pressure during measurement. In order for being advantageous with respect to such a pressing manner, in the first embodiment, a multiple reflecting ATR prism with a trapezoidal cross-section is employed.

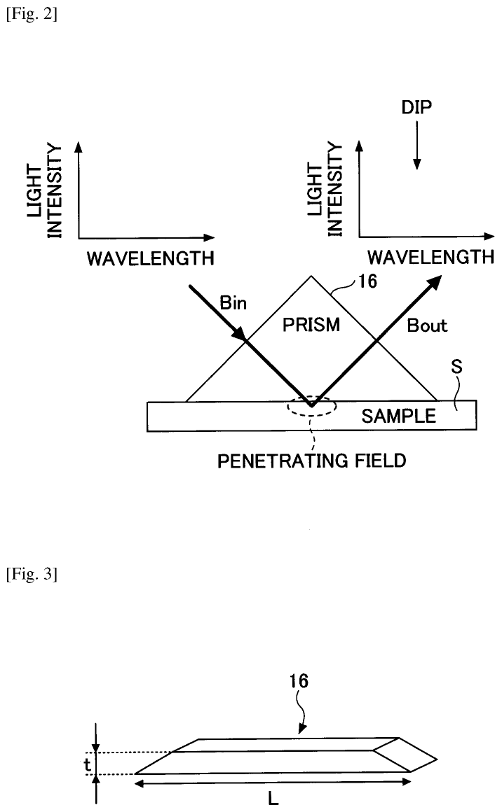

FIG. 3 is a perspective view depicting the structure of the ATR prism according to the first embodiment. As depicted in FIG. 3, the ATR prism 16 is a trapezoidal prism. The greater the number of multiple reflections in the ATR prism 16 becomes, the more sensitive the detection of glucose becomes. In addition, because a large contact area with a living body S can be achieved in this structure, a variation in a detection value due to a change in a pressure of pressing the ATR prism 16 to a living body S can be minimized. The length L of the bottom of the ATR prism 16 is, for example, 24 mm. The thickness t is set to cause multiple reflections, such as 1.6 mm or 2.4 mm.

As a material of the ATR prism 16, a material that is not toxic to a human body and exhibits a high transmission characteristic at a wavelength of about 10 μm, which is an absorption band of glucose, is a candidate. As an example, a ZnS (zinc sulfide) prism with a refractive index of 2.2 can be used, having great light penetration and being able to detect light deeply, from among the materials satisfying these conditions. ZnS, unlike ZnSe (zinc selenide), which is commonly used as an infrared material, is proved to be noncarcinogenic and used also as a non-toxic dye (lithopone) for a dental material.

In a typical ATR measuring apparatus, an ATR prism is fixed to a relatively large apparatus, so that a body part that is a to-be-measured object is limited to a surface of the body, such as a fingertip or a forearm. However, a skin at such an area is covered with a stratum corneum, about 20 μm thick, reducing the concentration of glucose detected. In addition, a stratum corneum is affected by a secretion of sweat or sebum, limiting the reproducibility of measurement. Therefore, in the blood glucose level measuring apparatus 100, the first hollow optical fiber 151 and the second hollow optical fiber 152 capable of transmitting probe light that is infrared light at low loss are used such that the respective ends are in contact with the ATR prism 16.

The first hollow optical fiber 151 is optically connected to the incidence face 161 of the ATR prism 16 at the one end in contact with the ATR prism 16 so that outgoing light from the first hollow optical fiber 151 is incident on the incidence face 161 of the ATR prism 16.

The second hollow optical fiber 152 is optically connected to the outgoing face 164 of the ATR prism 16 at the one end in contact with the ATR prism 16 so that outgoing light from the outgoing face 164 of the ATR prism 16 is guided to the second hollow optical fiber 152.

The ATR prism 16 allows for measurement of an earlobe where a blood capillary exists relatively near a skin surface and is less affected by sweat or sebum, as well as an oral mucosa that does not include keratin.

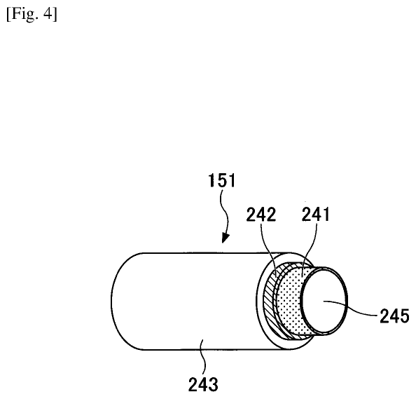

FIG. 4 is a perspective view illustrating an example of the structure of the hollow optical fiber used in the blood glucose level measuring apparatus 100. Mid-infrared light, which has a relatively long wavelength, for measuring glucose, is absorbed by glass in a quartz glass optical fiber and cannot be transmitted. Various types of optical fibers for infrared transmission using special materials have been developed, but problems of toxicity, hygroscopicity, and chemical durability of materials make these materials difficult to use in the medical field.

Each of the first hollow optical fiber 151 and the second hollow optical fiber 152 is such that, on an inner surface of a tube 243 formed of a non-harmful material such as glass, plastic, or the like, a metal thin film 242 and a dielectric thin film 241 are provided in the stated order. The metal thin film 242 is formed of a less toxic material, such as silver, and is coated with the dielectric thin film 241 to provide chemical and mechanical durability. In addition, because a core 245 is air that does not absorb mid-infrared light, low-loss transmission of mid-infrared light is possible over a wide wavelength range.

<Configuration of Processing Unit 2>

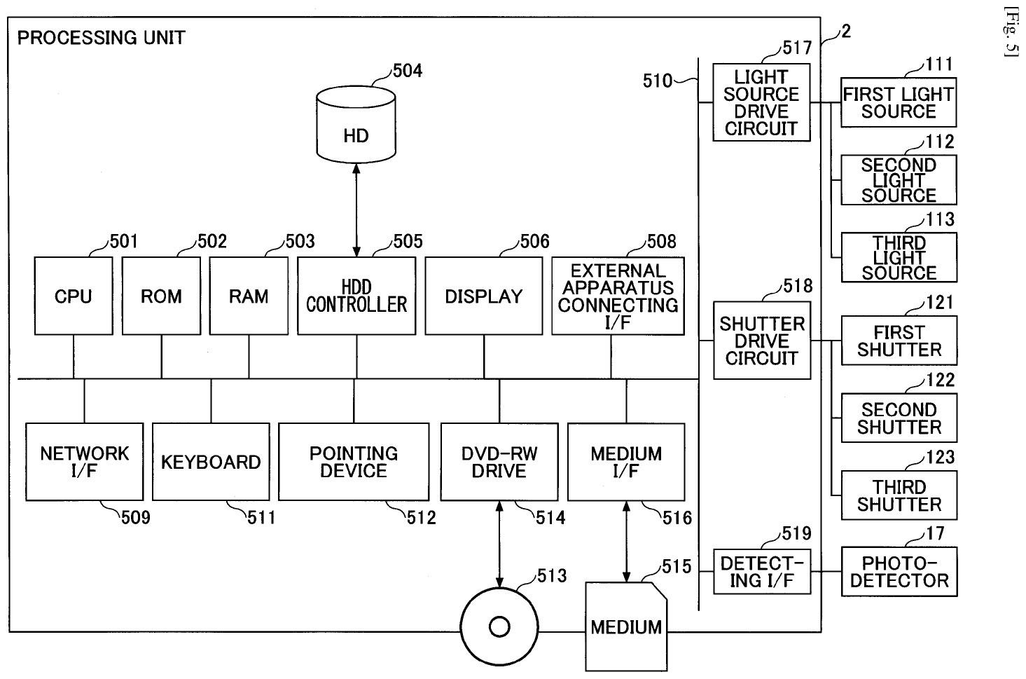

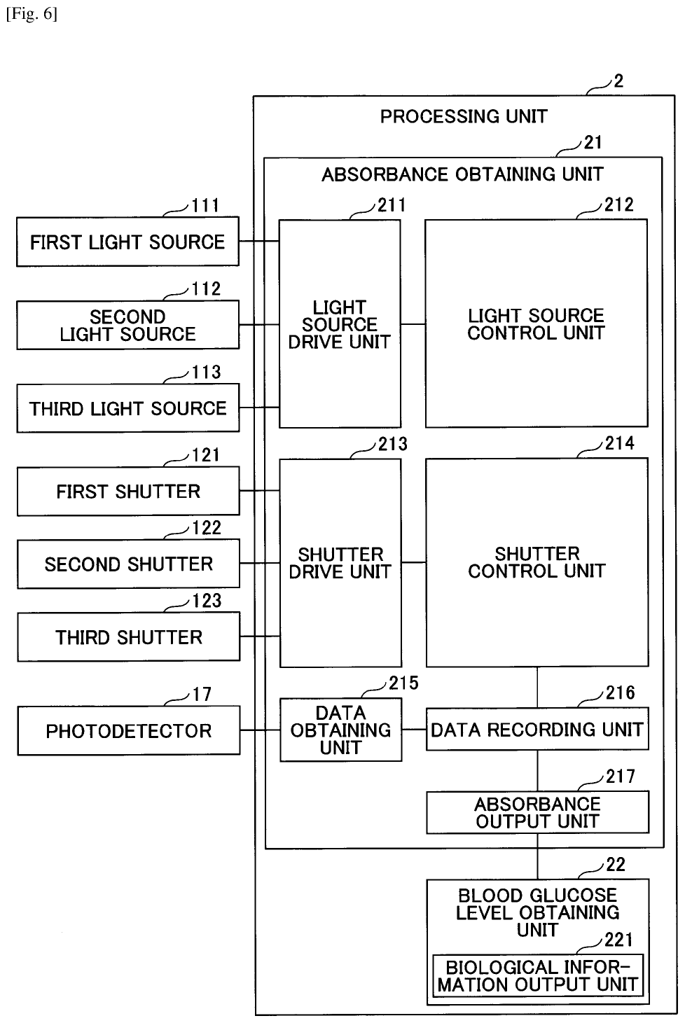

Next, the configuration of the processing unit 2 will be described with reference to FIGS. 5 and 6.

FIG. 5 is a block diagram illustrating an example of a hardware configuration of the processing unit 2 according to the first embodiment. As depicted in FIG. 5, the processing unit 2 includes a central processing unit (CPU) 501, a read-only memory (ROM) 502, a random access memory (RAM) 503, a hard disk (HD) 504, a hard disk drive (HDD), a HDD controller 505, and a display 506. The processing unit 2 also includes an external apparatus connecting interface (I/F) 508, a network I/F 509, a data bus 510, a keyboard 511, a pointing device 512, a digital versatile disk rewritable (DVD-RW) drive 514, a medium I/F 516, a light source drive circuit 517, a shutter drive circuit 518, and a detecting I/F 519.

The CPU 501 controls operation of the entire processing unit 2. The ROM 502 stores a program used to drive the CPU 501, such as an initial program loader (IPL). The RAM 503 is used as a work area of the CPU 501.

The HD 504 stores various data such as a program. The HDD controller 505 controls reading and writing of various data with respect to the HD 504 under the control of the CPU 501. The display 506 displays various information such as a cursor, a menu, a window, characters, and an image.

The external apparatus connecting I/F 508 is an interface for connecting with various external apparatuses. The external apparatuses may include, for example, a USB (Universal Serial Bus) memory and a printer. The network I/F 509 is an interface for performing data communication using a communication network. The bus line 510 includes an address bus, a data bus, and so forth for electrically connecting each element such as the CPU 501 depicted in FIG. 5.

The keyboard 511 is a type of an input unit with a plurality of keys for inputting of characters, numbers, various instructions, and the like. The pointing device 512 is a type of input unit for selecting and executing various instructions, selecting a processing target, moving a cursor, and the like. The DVD-RW drive 514 controls reading and writing of various data with respect to the DVD-RW 513 as an example of a removable recording medium. Instead of the DVD-RW, a DVD-R, or the like may be used. The medium I/F 516 controls reading and writing (storing) data with respect to the recording medium 515, such as a flash memory.

The light source drive circuit 517 is an electrical circuit, electrically connected to each of the first light source 111, the second light source 112, and the third light source 113 and, in response to a control signal, outputs a driving voltage to cause any light source to emit infrared light. The shutter drive circuit 518 is an electrical circuit, electrically connected to each of the first shutter 121, the second shutter 122, and the third shutter 123, and outputs a driving voltage that drives each shutter to open or close in response to a control signal.

The detecting I/F 519 is an electrical circuit such as an analog to digital (A/D) conversion circuit that serves as an interface for obtaining a detection signal of the photodetector 17. The detecting I/F 519 functions to obtain a detection signal not only from the photodetector 17, but also from various sensors, such as a pressure sensor or a temperature sensor, not depicted in FIG. 5.

FIG. 6 is a block diagram illustrating an example of a functional configuration of the processing unit 2 according to the first embodiment. As depicted in FIG. 6, the processing unit 2 includes an absorbance obtaining unit 21 and a blood glucose level obtaining unit 22.

The absorbance obtaining unit 21 includes a light source drive unit 211, a light source control unit 212, a shutter drive unit 213, a shutter control unit 214, a data obtaining unit 215, a data recording unit 216, and an absorbance output unit 217.

The functions of the light source drive unit 211 are implemented by the light source drive circuit 517, and the like, the functions of the shutter drive unit 213 are implemented by the shutter drive circuit 518, and the like, the functions of the data obtaining unit 215 are implemented by the detecting I/F 519, and the like, and the functions of the data recording unit 216 are implemented by the HD 504, and the like. The functions of the light source control unit 212, the shutter control unit 214, and the absorbance output unit 217 are implemented through execution of a predetermined program by the CPU 501, and the like.

The light source drive unit 211 outputs a driving voltage, on the basis of a control signal input from the light source control unit 212, to each of the first light source 111, the second light source 112, and the third light source 113, to emit infrared light. The light source control unit 212 controls timing and intensity of infrared light emission using the control signals.

The shutter drive unit 213 outputs a driving voltage on the basis of a control signal input from the shutter control unit 214 to open or close each of the first shutter 121, the second shutter 122, and the third shutter 123. The shutter control unit 214 controls timings and durations of opening the shutters by the control signals. The shutter control unit is an example of an incidence control unit.

The data obtaining unit 215 outputs, to the data recording unit 216, a detection value of light intensity obtained by sampling of a detection signal continuously output by the photodetector 17 at a predetermined sampling cycle. The data recording unit 216 stores the detection values input from the data obtaining unit 215.

The absorbance output unit 217 performs a predetermined calculation process on the basis of detection values read from the data recording unit 216 to obtain absorbance data and outputs the obtained absorbance data to the blood glucose level obtaining unit 22.

However, the absorbance output unit 217 may output obtained absorbance data to an external apparatus such as a PC through the external apparatus connecting I/F 508 or may output obtained absorbance data to an external server through the network I/F 509 and a network. Alternatively, obtained absorbance data may be output to the display 506 (see FIG. 5) for being displayed by the display 506.

The blood glucose level obtaining unit 22 includes a biological information output unit 221 as an example of an output unit. The biological information output unit 221 performs a predetermined calculation process on the basis of absorbance data input from the absorbance obtaining unit 21 to obtain the blood glucose level data, and outputs the obtained blood glucose level data to the display 506 for display.

However, the biological information output unit 221 may output blood glucose level data to an external apparatus such as a PC through the external apparatus connecting I/F 508 or may output blood glucose level data to an external server through the network I/F 509 and the network. The biological information output unit 221 may be configured to further output the reliability of blood glucose level measurement.

Because technology disclosed in Japanese Patent Application Laid-Open No. 2019-037752 or the like can be applied to the process of obtaining blood glucose level data from absorbance data, further details will be omitted.

<Example of Operation of Blood Glucose Level Measuring Apparatus 100>

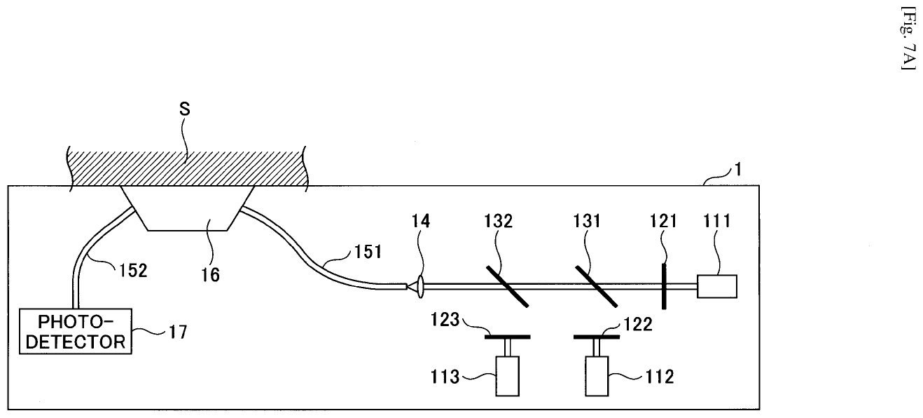

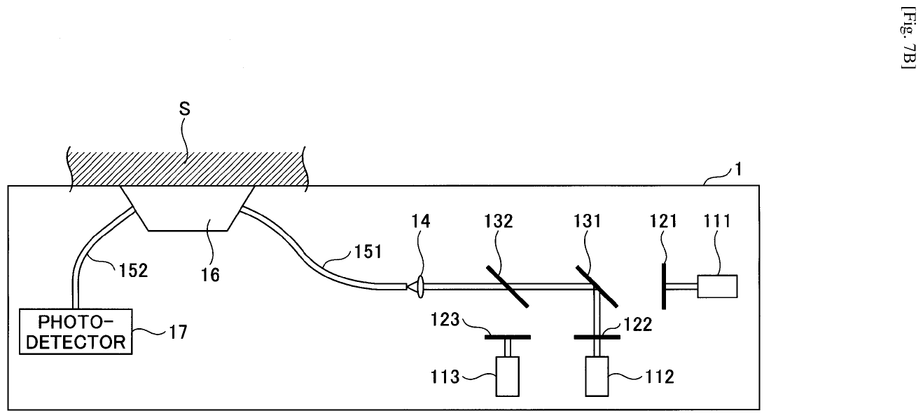

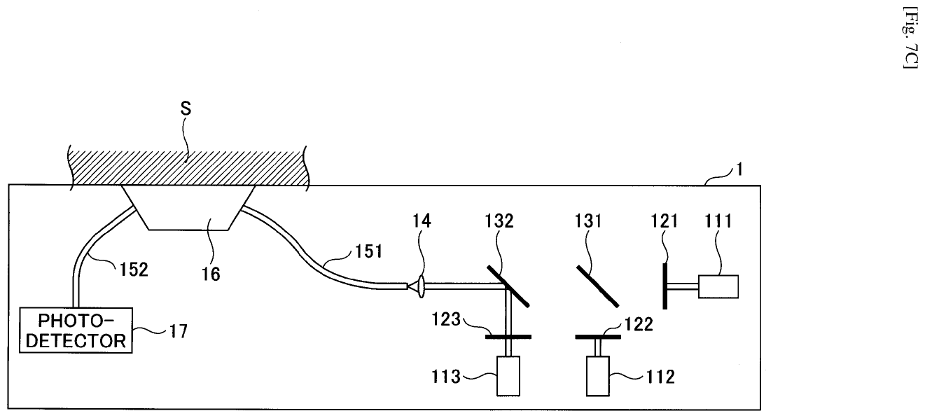

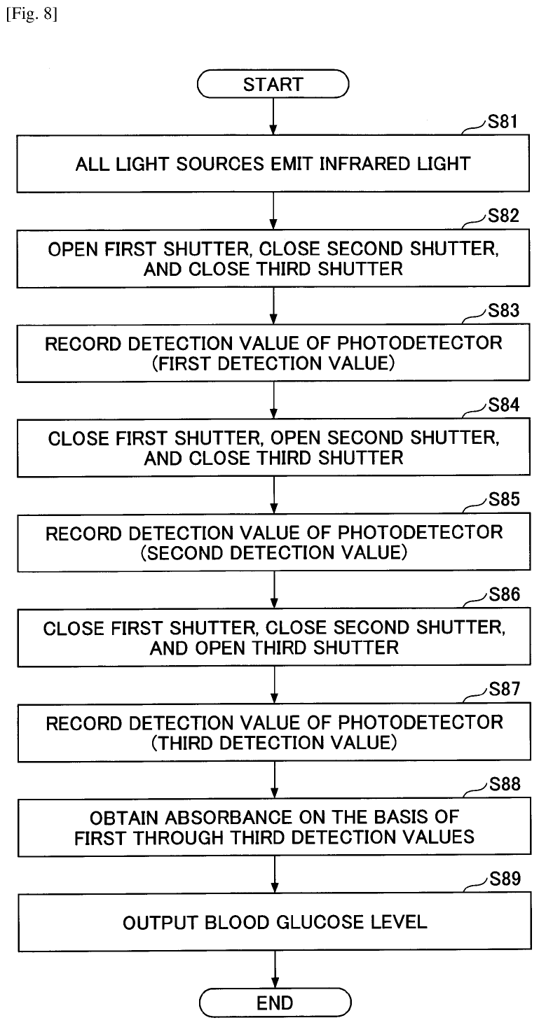

Next, operation of the blood glucose level measuring apparatus 100 will be described with reference to FIGS. 7A through 8.

(Example of Probe Light Switching Operation)

FIGS. 7A and 7B are diagrams for illustrating an example of a probe light switching operation. FIG. 7A depicts a state of the measuring unit 1 where first probe light is used. FIG. 7B depicts a state where second probe light is used. FIG. 7C depicts a state where third probe light is used.

In the first embodiment, the first light source 111, the second light source 112, and the third light source 113 emit infrared light at all times upon measuring absorbance and blood glucose levels, because incidence of probe light on the ATR prism 16 from each light source is controlled through opening and closing of the shutters.

As depicted in FIG. 7A, the first shutter 121 is open in response to a control signal. First probe light emitted by the first light source 111 passes through the first shutter 121 and is transmitted through each of the first and second half mirrors 131 and 132 to be guided to the first hollow optical fiber 151 via the coupling lens 14. Thereafter, after propagating through the first hollow optical fiber 151, the first probe light is incident on the ATR prism 16.

Because the second shutter 122 and the third shutter 123 are each closed, second probe light and third probe light are not incident on the ATR prism 16. Thus, in this state, absorbance with respect to the first probe light subject to attenuation at the ATR prism 16 is measured.

As depicted in FIG. 7B, the second shutter 122 is open in response to a control signal. Second probe light emitted by the second light source 112 passes through the second shutter 122, is reflected by the first half mirror 131, is transmitted through the second half mirror 132, and is guided to the first hollow optical fiber 151 via the coupling lens 14. Thereafter, after propagating through the first hollow optical fiber 151, the second probe light is incident on the ATR prism 16.

Because the first shutter 121 and the third shutter 123 are each closed, first probe light and third probe light are not incident on the ATR prism 16. Thus, in this state, absorbance with respect to the second probe light subject to attenuation at the ATR prism 16 is measured.

As depicted in FIG. 7C, the third shutter 123 is open in response to a control signal. Third probe light emitted by the third light source 113 passes through the third shutter 123, is reflected by the second half mirror 132, and is guided to the first hollow optical fiber 151 via the coupling lens 14. Thereafter, after propagating through the first hollow optical fiber 151, the third probe light is incident on the ATR prism 16.

Because the first shutter 121 and the second shutter 122 are each closed, first probe light and second probe light are not incident on the ATR prism 16. Thus, in this state, absorbance with respect to the third probe light subject to attenuation at the ATR prism 16 is measured.

When all of the first shutter 121, the second shutter 122, and the third shutter 123 are closed, none of first probe light, second probe light, and third probe light are incident on the ATR prism 16 and reach the photodetector 17.

In this manner, the shutter control unit 214 (see FIG. 6) as an incidence control unit can control opening and closing of each shutter to switch between a state in which first through third probe lights are sequentially incident on the ATR prism 16 and a state in which all of first through third probe lights are not incident on the ATR prism 16.

(Example of Operation of Blood Glucose Measuring Apparatus 100)

FIG. 8 is a flowchart depicting an example of operation of the blood glucose level measuring apparatus 100.

First, in step S81, in response to a control signal of the light source control unit 212, all of the first light source 111, the second light source 112, and the third light source 113 emit infrared light. However, in this initial state, the first shutter 121, the second shutter 122, and the third shutter 123 are all closed.

Subsequently, in step S82, the shutter control unit 214 opens the first shutter 121 and keeps the closed states of the second shutter 122 and the third shutter 123.

Subsequently, in step S83, the data recording unit 216 stores a detection value (a first detection value) of the photodetector 17 obtained by the data obtaining unit 215.

Subsequently, in step S84, the shutter control unit 214 opens the second shutter 122, closes the first shutter 121, and keeps the closed state of the third shutter 123.

Subsequently, in step S85, the data recording unit 216 stores a detection value (a second detection value) of the photodetector 17 obtained by the data obtaining unit 215.

Subsequently, in step S86, the shutter control unit 214 opens the third shutter 123, and keeps the closed state of the first shutter 121, and closes the second shutter 122.

Subsequently, in step S87, the data recording unit 216 stores a detection value (a third detection value) of the photodetector 17 obtained by the data obtaining unit 215.

Subsequently, in step S88, the absorbance output unit 217 obtains absorbance data with respect to the first through third probe lights on the basis of the first through third detection values and outputs the absorbance data to the biological information output unit 221.

Subsequently, in step S89, the biological information output unit 221 performs a predetermined calculation process on the basis of the absorbance data with respect to the first through third probe lights and obtains blood glucose level data. The obtained blood glucose level data is output to the display 506 (see FIG. 5) for display.

Thus, the blood glucose level measuring apparatus 100 can obtain and output blood glucose level data.

In the first embodiment, an example in which the first shutter 121, the second shutter 122, and the third shutter 123, which are electromagnetic shutters, are controlled to switch incident probe light on the ATR prism 16 is depicted, but incident light switching control is not limited to such a control manner. Incidence of probe light on the ATR prism 16 may be instead switched between turning on (emission) and turning off (not emission) of each of the plurality of light sources. In addition, a single light source that emits light of multiple wavelengths may be used to switch between incident light turning on and turning off for each wavelength.

In the first embodiment, the first half mirror and the second half mirror are used as elements that transmit a portion of probe light and reflect the rest. However, instead, also a beam splitter, a polarizing beam splitter, or the like may be used for the same purpose.

In addition, high refractive index materials, such as germanium, that transmit probe light have high surface reflectivity due to material characteristics. For example, when light (s-polarized) polarized in a vertical direction with respect to a direction of a surface of the substrate enters the substrate at an angle of incidence of 45 degrees, the ratio of transmission to reflection is approximately 1:1. Such characteristics may be used and a germanium plate may be installed in such a manner of implementing an angle of incidence of 45 degrees to replace the half mirror. In this regard, because also the back side has a 50% reflective component, an anti-reflection coating is applied to the back side.

<Variants of First Embodiment>

Hereinafter, variants of the first embodiment with respect to elements will be described.

(Control of Influence of Linearity Error of Photodetector 17)

The photodetector 17 used in the blood glucose level measuring apparatus 100 may include a linearity error, and the linearity error of the photodetector 17 may cause a blood glucose level measurement error. Therefore, probe light intensity can be changed to three or more predetermined levels to reduce the influence of linearity error by comparing probe light intensity with a detection value of the photodetector 17.

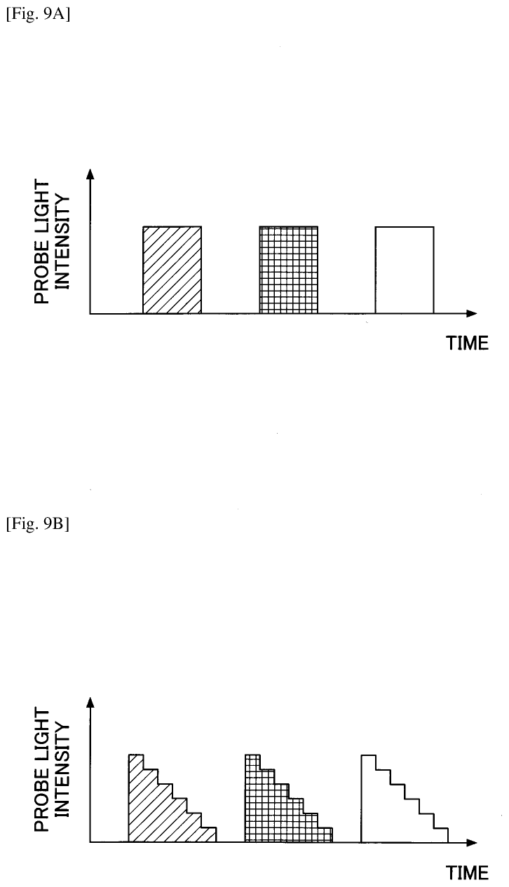

FIGS. 9A-9B are diagrams illustrating an example of probe light intensity changed in three or more levels as described above. FIG. 9A depicts probe light intensity in a comparative example. FIG. 9B depicts probe light intensity changed in three or more levels. In FIGS. 9A-9B, the portion indicated with diagonal hatching represents first probe light intensity, the portion indicated with lattice hatching represents second probe light intensity, and the portion indicated with no hatching represents third probe light intensity.

In FIG. 9A, light intensity of each probe light is constant, whereas, in FIG. 9B, light intensity of each probe light is gradually reduced in three or more levels. By changing a driving voltage or a driving current of the light source in three or more predetermined levels (six levels in FIG. 9B), emitted probe light intensity can be changed in three or more levels. It should be noted that light intensity of probe light in this case changes at a cycle shorter than the switching control cycle of probe light with respect to the shutter control unit 214 (for example, the cycle from step S82 through step S84 in FIG. 8).

In a case where the photodetector 17 does not have a linearity error, a detection value of the photodetector 17 varies linearly with a change in probe light intensity. In a case where the photodetector 17 has a linearity error, a detection value of the photodetector 17 varies non-linearly with a change in probe light intensity.

Therefore, probe light is emitted with a change in light intensity in three or more levels, a detection value of the photodetector 17 is obtained at each level, and the emitted probe light intensity data is compared with the detection value of the photodetector 17 to determine a light intensity range, in which linearity is ensured, from the detected light intensity varying in the three or more levels. Absorbance and blood glucose levels are measured using only the determined light intensity range in which linearity is ensured. Thus, it is possible to reduce the influence of the linearity error of the photodetector 17 to measure absorbance and blood glucose levels.

An operation to determine the light intensity range in which linearity is ensured may be performed prior to blood glucose level measurement or in a real-time manner during blood glucose level measurement.

Further, because there the plurality of (i.e., first through third) probe lights and the single photodetector 17 are used, the process of reducing the influence of linearity error of the photodetector 17 may be performed not using all of the plurality of probe lights, but may be performed using at least one of the plurality of probe lights.

(Detection of Probe Light by Image Sensor)

The photodetector 17 is not limited to a photodetector having a single pixel (a light receiving element), and may have a line-shaped image sensor in which pixels are arranged in line or an area-shaped image sensor in which pixels are arranged two-dimensionally.

Because a detection signal of the photodetector 17 is an integral value of received probe light intensity, if the optical path of incident light on or outgoing light from the ATR prism 16 is changed in response to a living body S touching the ATR prism 16, probe light intensity before and after the change is integrated, resulting in a detection error, and it may be impossible to obtain accurate absorbance data.

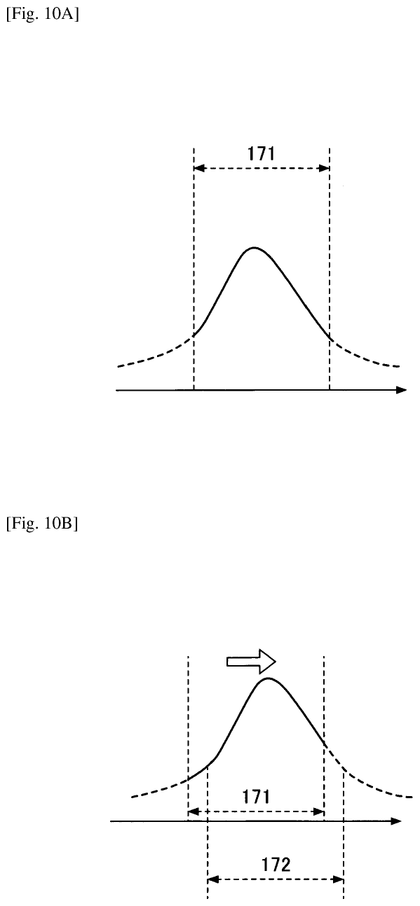

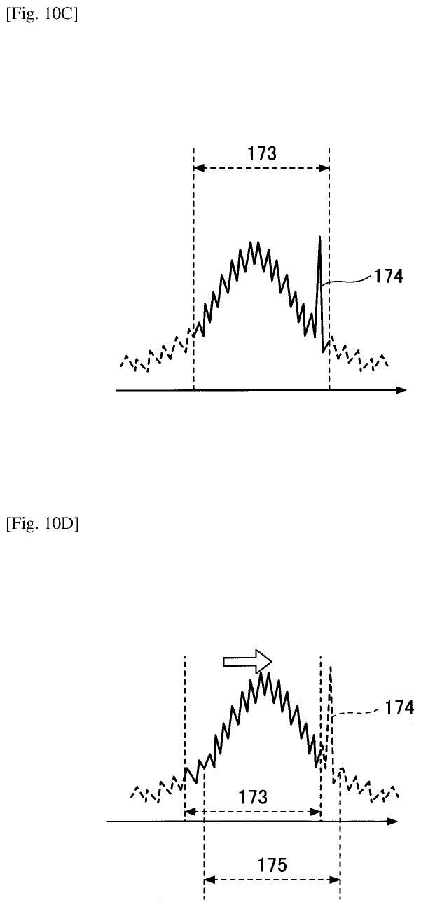

FIGS. 10A-10B depict such a probe light positional shift, and an area 171 is a light receiving area for probe light at the photodetector 17. As probe light shifts in a direction of an outlined arrow of FIG. 10B, the probe light intensity distribution in the area 171 changes, and the detection signal by photodetector 17 changes.

As a result of using an image sensor as the photodetector 17, a positional shift amount of probe light can be determined from a probe light image captured by the image sensor. Therefore, by using the integrated value of the probe light intensity distribution obtained after the shift as a detection signal, it is possible to correct the influence of positional shift of probe light. The area 172 of FIG. 10B depicts an area from which the integrated value of the probe light intensity distribution obtained after the positional shift is to be obtained.

When coherent light, such as laser light, is used as probe light, probe light may include a patchy light intensity distribution called a speckle. FIG. 10C depicts an example of a cross-sectional light intensity distribution of probe light including a speckle. FIG. 10C depicts an example of a singular point 174 of light intensity that may be included in a speckle image where the singular point 174 is included in an area 173.

FIG. 10D depicts a case where the probe light of FIG. 10C is shifted in the direction of the outlined arrow. Under the condition, the singular point 174 is no longer included in the area 173, and the change in the detection signal before and after the shift becomes significant. By using the integrated value of the probe light intensity distribution in the area 175 as a detection signal appropriately depending on the probe light positional shift amount that can be determined from the probe light image, it is possible to more desirably reduce the influence of the probe light positional shift.

In addition, it is possible to reduce a measurement variation error by estimating the contact area between a living body S and the ATR prism 16 on the basis of a probe light intensity distribution obtained by the image sensor, and correcting the detection value on the basis of the detection signal of the image sensor using an in-plane sensitivity distribution of the ATR prism 16 previously obtained and stored before the start of measurement.

(Incidence Face to Total Reflection Member)

In the first embodiment described above, the incidence face 161 of the ATR prism 16 is planar, but is not limited to be planar, and may have any one of various shapes, such as a surface having a diffusing surface or a surface having a curvature.

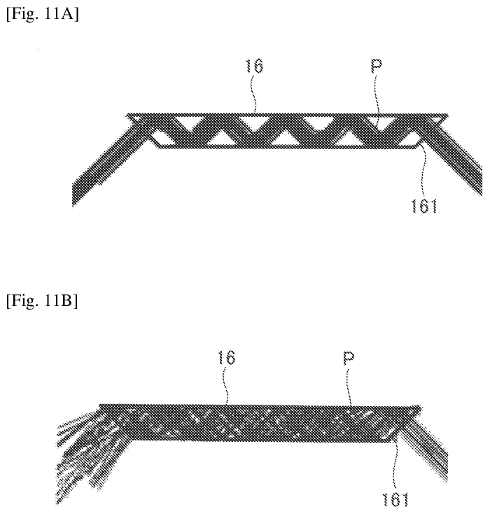

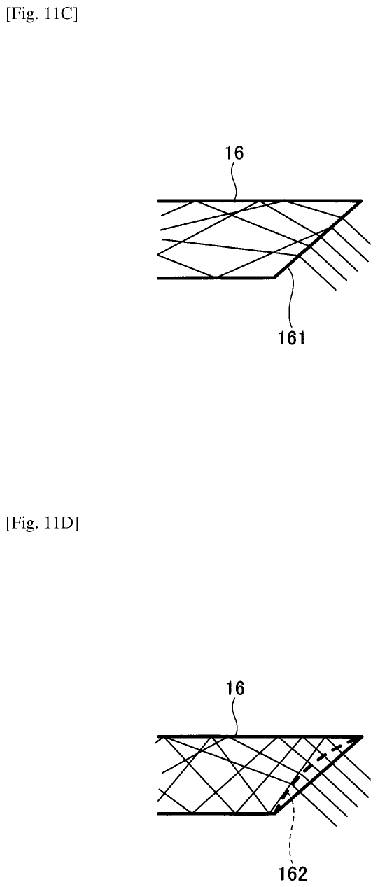

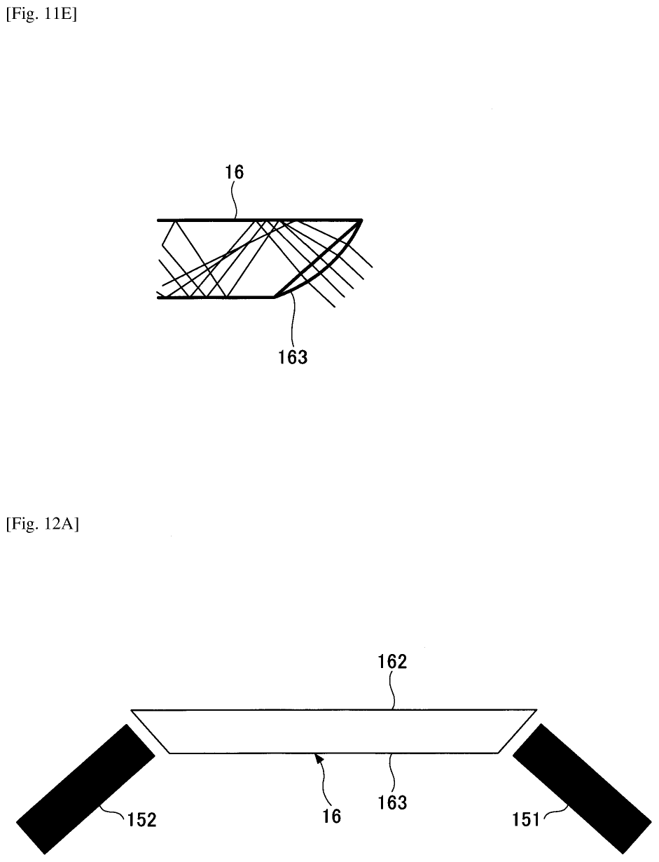

As depicted in FIG. 11A, when the incidence face 161 is planar, the directions of propagation of probe light in the ATR prism 16 are uniform in accordance with the angle of incidence on the incidence face 161. For this reason, there may be an area dependence (there may be a different measurement sensitivity for each area) in the total reflection face of the ATR prism 16 in contact with a living body S.

A detection signal of the photodetector 17 depends on a contact state, such as the size of a contact area of a living body S in contact with the ATR prism 16. In particular, when a living body S, such as a lip or a finger, is a to-be-measured object, the reproducibility of a contact state tends to be low, and a measurement variation may increase due to the area dependence of measurement sensitivity.

On the other hand, by changing the directions of propagation of probe light in the ATR prism 16 randomly by using the incidence face 161 having a diffusing surface, the area dependence of measurement sensitivity can be reduced and the measurement variation can be reduced, as depicted in FIG. 11B.

Other than a diffusing surface illustrated in FIG. 11C, the incidence face 161 may have a concave surface or a protruded surface as illustrated in FIG. 11D or a convex surface or a hollow surface as illustrated in FIG. 11E. The concave or protruded surface in FIG. 11D and the convex or hollow surface in FIG. 11E are examples of an incidence face having curvature. In this case, the optical paths of probe light can be changed as in the above-described case of using the diffusing surface, and a measurement variation can be reduced by reducing the area dependence of measurement sensitivity.

The same effect can be obtained by placing a diffusing plate or a lens on the optical path before probe light is incident on the ATR prism 16. However, in this case, the increase in the number of elements of the blood glucose level measuring apparatus may lead to a difference (apparatus difference) in a measurement value depending on each apparatus due to an assembly error or lead to an increase in the cost. Using a diffusing surface or a curved surface as the incidence face 161 of the ATR prism 16 is more suitable because such an apparatus dependence or a cost increase can be avoided.

(Supports of light guide and total reflection member)

When the first hollow optical fiber 151 and the second hollow optical fiber 152 are shifted relative to the ATR prism 16 in response to a living body S touching the ATR prism 16, the incident and outgoing efficiency of probe light with respect to the ATR prism 16 may vary, and a measurement variation may increase.

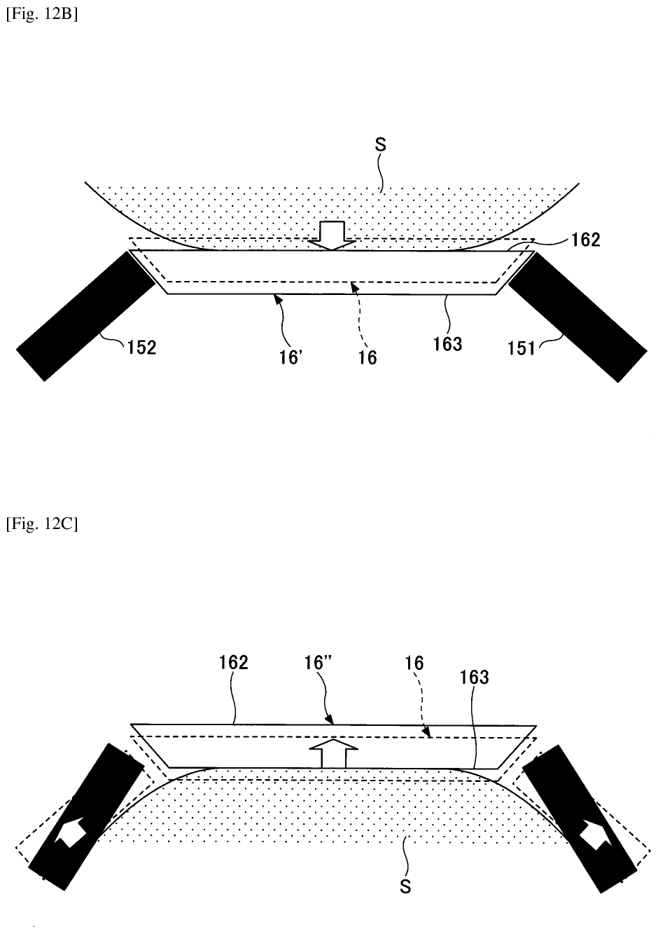

FIGS. 12A-12C are diagrams illustrating such a relative shift of the first hollow optical fiber 151 and the second hollow optical fiber 152 with respect to the ATR prism 16. FIG. 12A depicts a case where the ATR prism 16 is not in contact with a living body S. FIG. 12B depicts a case where a living body S is in contact with the first total reflection face 162 of the ATR prism 16. FIG. 12C depicts a case where a living body S is in contact with the second total reflection face 163 of the ATR prism 16.

As depicted in FIG. 12B, when a living body S contacts the first total reflection face 162 of the ATR prism 16, a pressure is applied downward as indicated by an outline arrow, causing the ATR prism 16 to shift downward. As a result, the ATR prism 16 enters the state of the ATR prism 16′, and the positions of the first hollow optical fiber 151 and the second hollow optical fiber 152 relative to the ATR prism 16′ change accordingly.

As depicted in FIG. 12C, when a living body S contacts the second total reflection face 163 of the ATR prism 16, a pressure is applied upward as indicated by an outline arrow, causing the ATR prism 16 to shift upward. As a result, the ATR prism 16 enters the state of the ATR prism 16″, and the positions of the first hollow optical fiber 151 and the second hollow optical fiber 152 relative to the ATR prism 16″ change accordingly.

Such a relative shift causes a variation in the incident and outgoing efficiency of probe light with respect to the ATR prism 16. Especially, when a to-be-measured object is a living body, because it is not easy to maintain the constant contact pressure, a measurement variation due to a relative shift is particularly likely to increase.

Accordingly, the first hollow optical fiber 151, the second hollow optical fiber 152, and the ATR prism 16 are desirably supported by the same support in order to avoid a relative shift.

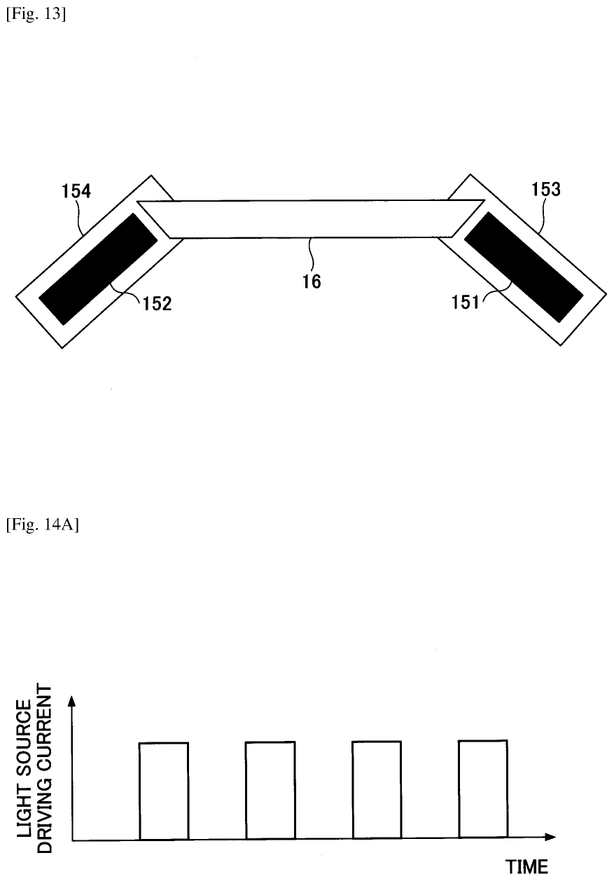

FIG. 13 is a diagram illustrating an example of a configuration of a member supporting the first hollow optical fiber 151, the second hollow optical fiber 152, and the ATR prism 16. A light guide support 153 of FIG. 13 is a member that integrally supports the first hollow optical fiber 151 and the ATR prism 16. An outgoing support 154 is a member that integrally supports the second hollow optical fiber 152 and the ATR prism 16.

As a result of the first hollow optical fiber 151 and the ATR prism 16 being thus integrally supported, when a living body S comes into contact with the ATR prism 16, these two elements move together, so that a relative shift does not occur between these elements. In addition, as a result of the second hollow optical fiber 152 and the ATR prism 16 being thus integrally supported, when a living body S comes into contact with the ATR prism 16, these elements move together, so that a relative shift does not occur between these elements. Therefore, a variation in the incident efficiency or the outgoing efficiency of probe light caused by contact of a living body S with the ATR prism 16 can be reduced, and the measurement variation can be reduced.

With regard to the above example, the light guide support 153 and the outgoing support 154 are described as being separate members. However, the first hollow optical fiber 151, the second hollow optical fiber 152, and the ATR prism 16 may be supported by a single support.

In addition, even in a case where the light guide is implemented by an optical element such as a mirror or a lens without using the first hollow optical fiber 151, the same advantageous effect as described above can be obtained by supporting the optical element and the ATR prism 16 together.

Further, not only the light guide but also the first light source 111, the second light source 112, the third light source 113, and the photodetector 17 may be integrally supported by the same support member, so that the measurement variation can be reduced.

(Radio Frequency Modulation of Light Source Driving Current)

If probe light includes a speckle, a detection value of the photodetector 17 may vary depending on the pattern of the speckle to increase the measurement variation. Because such a speckle is generated due to interference of scattered light of probe light or the like, generation of a speckle can be reduced by reducing the coherence of probe light. Therefore, in the first embodiment, by superimposing a high frequency modulation component with a current driving a light source, the coherence of the light source included in the blood glucose level measuring apparatus can be reduced, and the measurement variation in absorbance due to a speckle of probe light can be reduced.

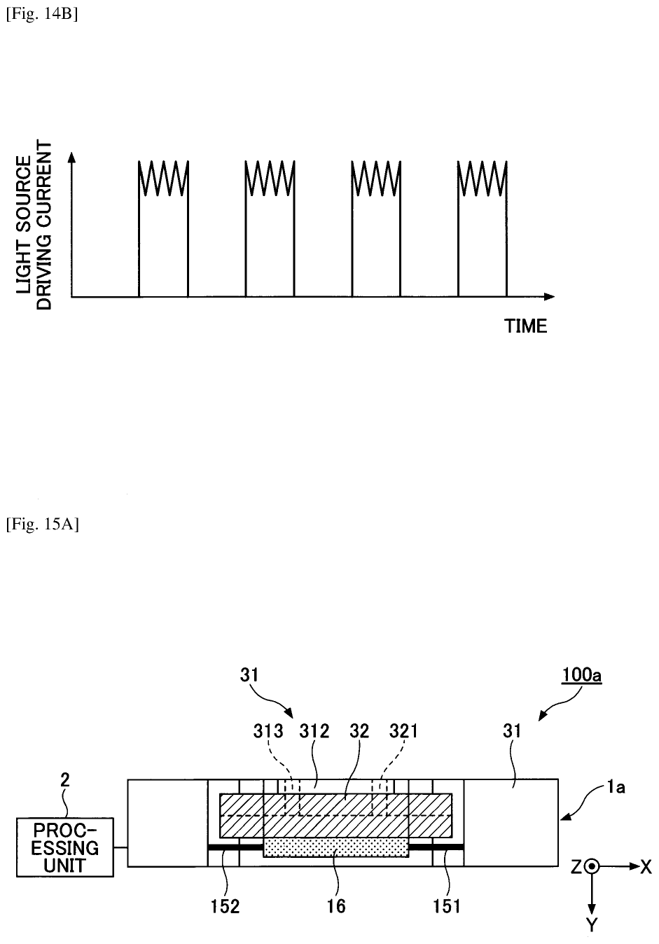

FIGS. 14A and 14B are diagrams illustrating an example of a light source driving current. FIG. 14A depicts a light source driving current according to a comparative example. FIG. 14B depicts a light source driving current with high frequency modulation.

The light source control unit 212 (see FIG. 6) periodically outputs a pulsed driving current as depicted in FIG. 14A to each of the first light source 111, the second light source 112, and the third light source 113 to cause the light source to emit pulsed probe light.

In the first embodiment, a high frequency modulation component is superimposed on the pulsed driving current of FIG. 14A to output to each of the first light source 111, the second light source 112, and the third light source 113. The waveform of the high frequency modulation component may be sinusoidal or rectangular. The modulation frequency can be any one selected from among the range from 1 MHz (megahertz) to several GHz (gigahertz).

By superimposing a high frequency modulation component, each of the first light source 111, the second light source 112, and the third light source 113 emits pseudo multimode laser light as probe light, to reduce the coherence of the probe light. This reduces generation of a speckle of probe light by reducing the coherence and reduces the measurement variation caused by a speckle.

### Second Embodiment

Next, a blood glucose level measuring apparatus according to a second embodiment will now be described. In this regard, the same reference numerals are given to elements identical or corresponding to elements of the first embodiment described above, and duplicate descriptions for these elements may be omitted.

In the second embodiment, a light source emitting probe light, a total reflection member in contact with a to-be-measured object and causing total reflection of incident probe light, a light intensity detector detecting the light intensity of the probe light exiting from the total reflection member, and an output unit outputting blood glucose level information obtained on the basis of the light intensity are provided. In addition, a first support is provided to support the light source and the light intensity detector, and a second support is detachably provided to the first support to support the total reflection member.

With the configuration, it is possible to provide a blood glucose level measuring apparatus which ensures safety while reducing the cost of the blood glucose level measuring apparatus by only exchanging the total reflection member that is to be in contact with a living body without exchanging the light source and the light intensity detector.

<Example of Configuration of Blood Glucose Level Measuring Apparatus 100a>

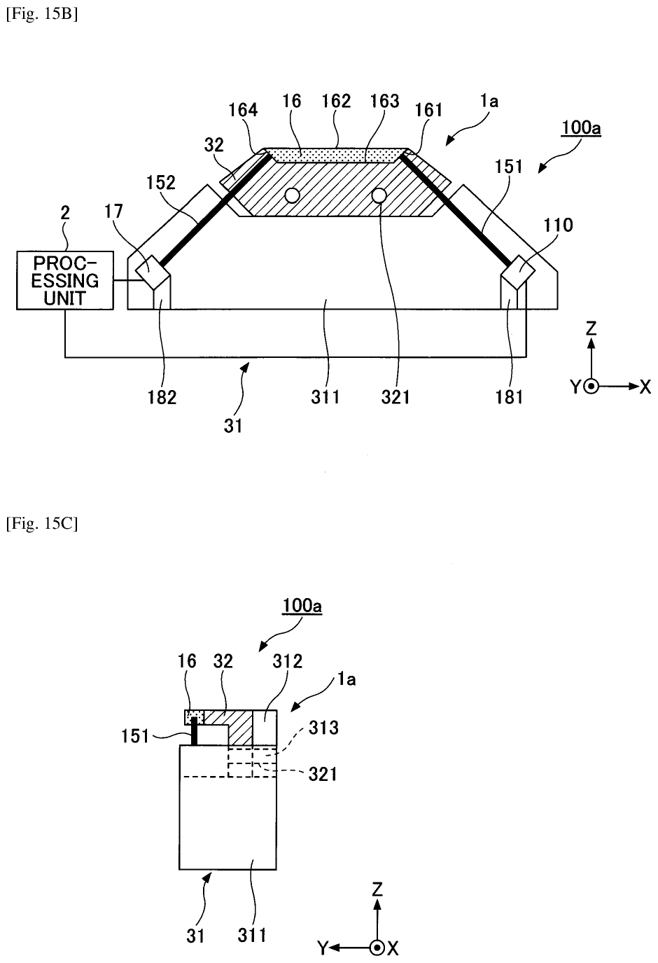

First, the configuration of the blood glucose level measuring apparatus 100a according to the second embodiment will be described. FIGS. 15A-15C are diagrams illustrating an example of the configuration of the blood glucose level measuring apparatus 100a. FIG. 15A is a top view of the blood glucose level measuring apparatus 100a. FIG. 15B is a front view of the blood glucose level measuring apparatus 100a. FIG. 15C is a side view of the blood glucose level measuring apparatus 100a.

As depicted in FIGS. 15A-15C, the blood glucose level measuring apparatus 100a includes a measuring unit la, and the measuring unit la includes a first support 31, a quantum cascade laser (QCL) 110, and a second support 32. The second support 32 is detachable from the first support 31. FIGS. 15A-15C depict a state where the second support 32 is mounted to the first support 31.

The first support 31 includes a box-shaped member 311 and a back plate 312. The box-shaped member 311 is a member that supports, in the inside, the QCL 110, first hollow optical fiber 151, second hollow optical fiber 152, and photodetector 17. The back plate 312 is fixed to the +Z side surface of the box-shaped member 311 and functions of connecting with the second support 32. The front view of FIG. 15B depicts the inside of the box-shaped member 311 in a see-through view.

In the box-shaped member 311, a light source support 181 and a photodetector support 182 are fixed at a +Z side of the bottom plate inside. On a slope of the light source support 181, the QCL 110 is fixed, and, on a slope of the photodetector support 182, the photodetector 17 is fixed. The fixing may be implemented by adhesive, screws, or the like. The same manner will apply to the following cases where the term “fix” is used with regard to the second through fourth embodiments.

The QCL 110 is a variable wavelength quantum cascade laser that emits laser light of 1050 cm⁻¹ as first probe light, emits laser light of 1070 cm⁻¹ as second probe light, and emits laser light of 1100 cm⁻¹ as third probe light.

Thus, in the second embodiment, the QCL 110 has the functions of the first light source 111, the second light source 112, and the third light source 113 described above (see FIG. 1) with regard to the first embodiment. In the second embodiment, because emission of first through third probe lights by the QCL 110 can be switched by a control signal, the configurations for switching the wavelengths such as the first shutter 121, the second shutter 122, the third shutter 123, the first half mirror 131, and the second half mirror 132 in FIG. 1 are omitted. Hereinafter, the first through third probe lights are generally referred to as probe light P.

The first hollow optical fiber 151 is supported by the QCL 110 in such a manner that one end is fixed to the QCL 110 to enable probe light P to be guided to the QCL 110. A portion of the first hollow optical fiber 151 at a side connected to the QCL 110 in the length direction is held inside the first support 31. The remaining portion of the first hollow optical fiber 151 protrudes from the first support 31 toward the ATR prism 16, and the protruding end is in contact with the incidence face 161 of the ATR prism 16. However, the protruding end is not fixed to the ATR prism 16, and the ATR prism 16 can be spaced from the first hollow optical fiber 151.

The second hollow optical fiber 152 is supported by the photodetector 17 in such a manner that one end is fixed to the photodetector 17 to enable probe light P to be guided to the photodetector 17. A portion of the second hollow optical fiber 152 at a side connected to the photodetector 17 in the length direction is held inside the first support 31. The remaining portion of the second hollow optical fiber 152 protrudes from the first support 31 toward the ATR prism 16, and the protruding end is in contact with the outgoing face 164 of the ATR prism 16. However, the protruding end is not fixed to the ATR prism 16, and the ATR prism 16 can be spaced from the second hollow optical fiber 152.

As depicted in FIG. 15C, the second support 32 is an L-shaped member viewed from the X direction side, and an end of the −Z direction side end of the L-shape is in contact with the upper face of the box-shaped member 311. Two through holes 321 extending in the Y direction and arranged in the X direction are provided in a planar section of the second support 32 extending along a XZ plane. Two tap holes 313 are provided in the back plate 312 of the first support 31 at positions corresponding to the through holes 321, respectively.

The +Y direction end of the L-shape of the second support 32 is in contact with the face of the ATR prism 16 at the −Y direction side, and the ATR prism 16 is fixed to the second support 32. The second support 32 is thus fixed to the lateral side face of the ATR prism 16 to support the ATR prism 16.

The +Y and −Y side faces of the ATR prism 16 are orthogonal to the first total reflection face 162 and the second total reflection face 163 of the ATR prism 16, respectively. The −Y side face of the ATR prism 16 corresponds to “a lateral side face orthogonal to a total reflection face of a total reflection member”.

The through holes 321 are formed at positions to have predetermined relationships with the ATR prism 16 supported by the second support 32. More specifically, when the position of the vertex formed by the incidence face 161 and the first total reflection face 162 of the ATR prism 16 is used as a positional reference, the through holes 321 are formed at positions to have predetermined relationships with the position of the vertex when the second support 32 supports the ATR prism 16.

Accordingly, when the second support 32 is mounted to the first support 31 so that the through holes 321 and the tap holes 313 are aligned, the ATR prism 16 is positioned at a predetermined position with respect to the first support 31, and the first total reflection face 162 and the second total reflection face 163 are positioned at predetermined positions with respect to the first support 31. Each of the two through holes 321 is an example of a to-be-coupled unit, and each of the two tap holes 313 is an example of a coupling unit.

When the second support 32 is to be mounted to the first support 31, the second support 32 is lowered in the −Z direction so that the −Z direction end of the L-shape of the second support 32 is caused to be in contact with the upper face of the box-shaped member 311. In addition, the face of the second support 32 at the −Y direction side is caused to be in contact with the face of the back plate 312 at the +Y direction side.

In this state, the second support 32 is more finely aligned so that the two tap holes 313 in the back plate 312 are aligned with the two through holes 321 in the second support 32. Then, in the thus aligned state, the second support 32 and the first support 31 are connected as a result of screws being inserted through the two through holes 321, respectively, and the thus inserted screws being then threaded through the tap holes 313, respectively. The second support 32 can be thus mounted to the first support 31.

The positions of the first hollow optical fiber 151 and the second hollow optical fiber 152 are predetermined in such a manner that, when the second support 32 is thus mounted to the first support 31, the end of the first hollow optical fiber 151 is in contact with the incidence face 161 of the ATR prism 16 and the end of the second hollow optical fiber 152 is in contact with the incidence face 161 of the ATR prism 16.

In FIG. 15, indication of the screws that are inserted through the through holes 321 and threaded through the tap hole 313 is omitted.

<Functions and Effects of Blood Glucose Level Measuring Apparatus 100a>

In measurement of biological information such as a blood glucose level using a total reflection member such as the ATR prism 16, a lip of a to-be-measured person as a to-be-measured object is caused to be in contact with at least one of the first total reflection face 162 and the second total reflection face 163 of the ATR prism 16 for measurement.

This contact may cause residue or dust to adhere to the first total reflection face 162 or the second total reflection face 163, or cause scratches, which may result in inability to accurately detect the attenuation of probe light by a to-be-measured person, and thus make it impossible to accurately measure a blood glucose level. In addition, it may be undesirable in terms of safety and hygiene to use a blood glucose level measuring apparatus in multiple to-be-measured persons because the lips or the like of the to-be-measured persons touch the total reflection faces.

Therefore, it is desirable to detach a part of a measuring apparatus and perform maintenance such as cleaning and replacement. In the related art, there is disclosed an apparatus in which a part of a measuring apparatus is detachable and a light source such as a light emitting element, an optical part such as a light waveguide, and a photodetector such as a light receiving element are formed on a substrate and interchangeable.

However, the related art may increase the cost of the blood glucose level measuring apparatus by replacing the light source, the optical part, and the photodetector together. The higher cost of the blood glucose level measuring apparatus is more remarkable because light sources and photodetectors corresponding to the mid-infrared region of probe light, particularly these devices suitable for blood glucose level measurement, are expensive.

In contrast, in the present embodiment, the first support 31 supports the QCL 110 that emits probe light P and the photodetector 17 that detects the light intensity of the probe light P exiting from the ATR prism 16, and the second support 31 is detachably mounted to the first support 31 to support the ATR prism 16.

This allows the ATR prism 16 to be replaced without replacing the QCL 110 and the photodetector 17. By not replacing the QCL 110 and the photodetector 17, the cost of the blood glucose level measuring apparatus can be reduced, and the ATR prism 16 can be replaced to ensure safe and hygienic conditions. Thus, the blood glucose level measuring apparatus that is safe while reducing the cost of the blood glucose level measuring apparatus can be provided.

Also in the present embodiment, the second support 32 supports the ATR prism 16 at the −Y direction side face which is one of the faces of the ATR prism 16 orthogonal to the first total reflection face 162 or the second total reflection face 163 of the ATR prism 16.

By thus supporting the ATR prism 16, when a to-be-measured person's lip is caused to be in contact with the first or second total reflection face 162 or 163 for a measurement, the to-be-measured person can cause the to-be-measured person's mouth to face the +Y direction side of the ATR prism 16 and cause the lip to be in contact with the first or second total reflection face 162 or 163. Also, the incidence face 161, first total reflection face 162, second total reflection face 163, and outgoing face 164 are not used to support the ATR prism 16. Accordingly, a blood glucose level can be accurately measured using the to-be-measured person's lip as a to-be-measured object without interfering with the functions of the ATR prism 16.

In the present embodiment, the through holes 321 are formed in predetermined positional relationships with the ATR prism 16 supported by the second support 32. Therefore, when the through holes 321 and the tap holes 313 are aligned and the second support 32 is mounted to the first support 31, the ATR prism 16 is positioned at a predetermined position with respect to the first support 31, and the first total reflection face 162 and the second total reflection face 163 are positioned at predetermined positions with respect to the first support 31. This ensures reproducibility of a blood glucose level measurement by placing the ATR prism 16 at the same position at any occasion although the second support 32 is replaced with respect to the first support 31.

Also in the present embodiment, the first support 31 supports, together, the QCL 110 and the photodetector 17 as well as the first hollow optical fiber 151 as a light guide. This allows probe light P emitted by the QCL 110 to be appropriately guided toward the ATR prism 16 to enable a proper measurement of a blood glucose level.

In the present embodiment, as a method of coupling the first support 31 with the second support 32, the through holes 321 and the tap holes 313 are connected through the screws, but a specific connection method is not limited to the above-described method. For example, knock pins (fitting units) may be provided in place of the tap holes 313 on the back plate 312 of the first support 31, and knock holes (to-be-fitted units) may be provided in place of the through holes 321 in the planar section of the second support 32 extending along the XZ plane. The knock pins may be fitted to the knock holes to couple the first support 31 with the second support 32.

### Third Embodiment

Next, a blood glucose level measuring apparatus 100b according to a third embodiment will now be described. In this regard, the same reference numerals are given to elements identical or corresponding to elements of the first embodiment described above, and duplicate descriptions for these elements may be omitted.

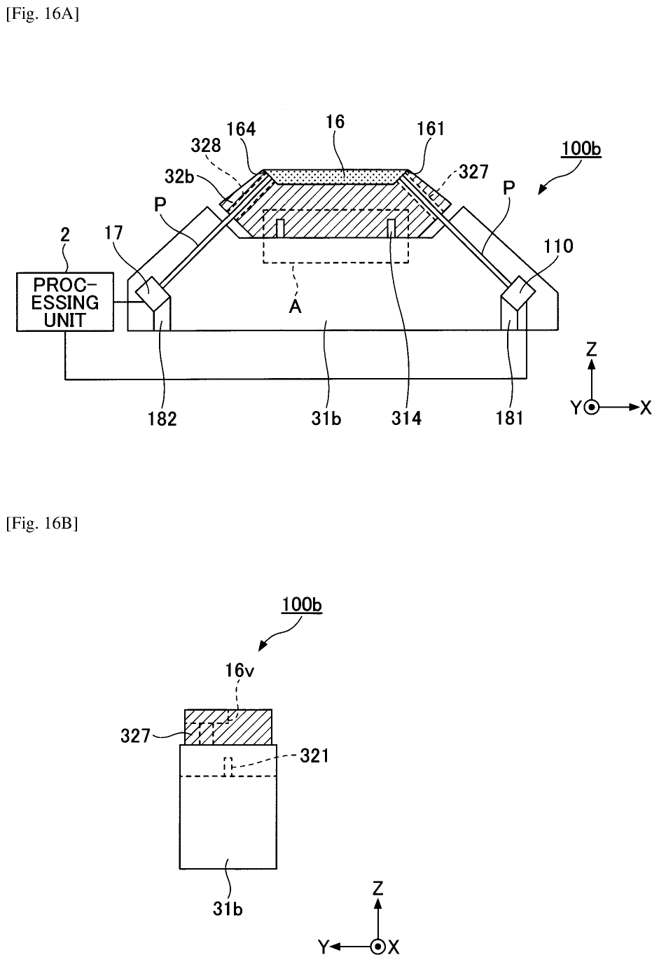

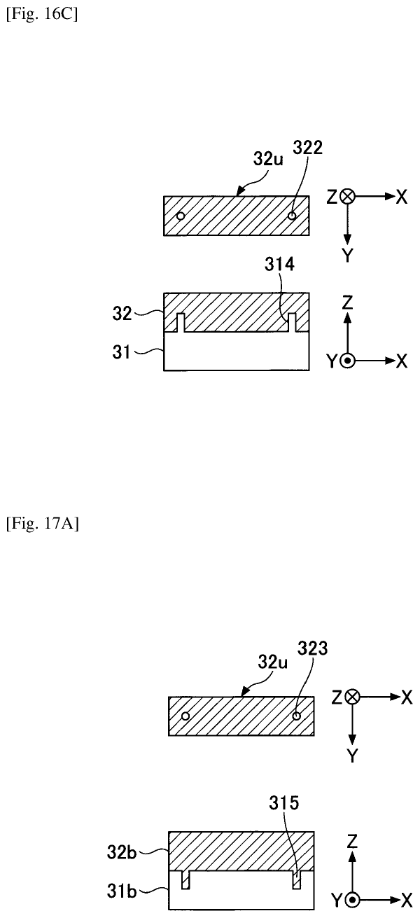

FIGS. 16A-16C are diagrams illustrating an example of a configuration of the blood glucose level measuring apparatus 100b. FIG. 16A is a front view, FIG. 16B is a side view, and FIG. 16C is a detailed view of the part A (a part enclosed by a broken line) in FIG. 16A. FIG. 16A depicts the blood glucose level measuring apparatus 100b in a see-through view. The same manner will apply to the front views of the following figures with regard to the third and fourth embodiments. In FIG. 16C, a view 32u depicts a second support 32b viewed from the −Z direction side.

As depicted in FIGS. 16A-16C, the blood glucose level measuring apparatus 100b includes a first support 31b and the second support 32b.

The first support 31b is a box-shaped member that supports the QCL 110, the first hollow optical fiber 151, the second hollow optical fiber 152, and the photodetector 17. In the first support 31b, a light source support 181 and a photodetector support 182 are fixed at the +Z side of the bottom plate inside. On a slope of the light source support 181, the QCL 110 is fixed, and on a slope of the photodetector support 182, the photodetector 17 is fixed. In addition, two knock pins 314 are provided on the +Z direction side face of the first support 31b.

The second support 32b is a block-shaped member having a hexagonal shape viewed from the Y direction side. The second support 32b is provided with a recess 16v for inserting and fixing the ATR prism 16, an incidence through hole 327 through which probe light P is incident on the ATR prism 16, and an outgoing through hole 328 through which probe light P exits from the ATR prism 16. In addition, two knock holes 322 corresponding to the two knock pins 314 of the first support 31b, respectively, are formed from the face of the second support 32b at the −Z direction side.

As depicted in FIG. 16A, the incidence through hole 327 is a diagonally passing through hole and is formed in such a manner that probe light P from the QCL 110 reaches the incidence face 161 of the ATR prism 16. The outgoing through hole 328 is also a diagonally passing through hole and is formed in such a manner that probe light P exiting from the ATR prism 16 reaches the photodetector 17.

The knock holes 322 in the second support 32b are formed in a predetermined positional relationship with the ATR prism 16 supported by the second support 32b. Therefore, when the second support 32b is mounted to the first support 31b so that the knock pins 314 and the knock holes 322 fit together, the ATR prism 16 is positioned in a predetermined position with respect to the first support 31b, and the first total reflection face 162 and the second total reflection face 163 are positioned in predetermined positions with respect to the first support 31b. Each of the two knock holes 322 is an example of a to-be-coupled unit, and each of the two knock pins 314 is an example of a coupling unit.

When the second support 32b is to be mounted to the first support 31b, the second support 32b is lowered in the −Z direction to implement fitting of the two knock holes 322 and the two knock pins 314 together. Thus, the second support 32b can be mounted to the first support 31b.

The configuration of the blood glucose level measuring apparatus 100b allows easy mounting of the second support 32b to the first support 31b. The other advantageous effects are the same as the corresponding advantageous effects described above for the second embodiment.

With regard to the present embodiment, variants will now be described for the configuration of coupling the second support 32b to the first support 31b.

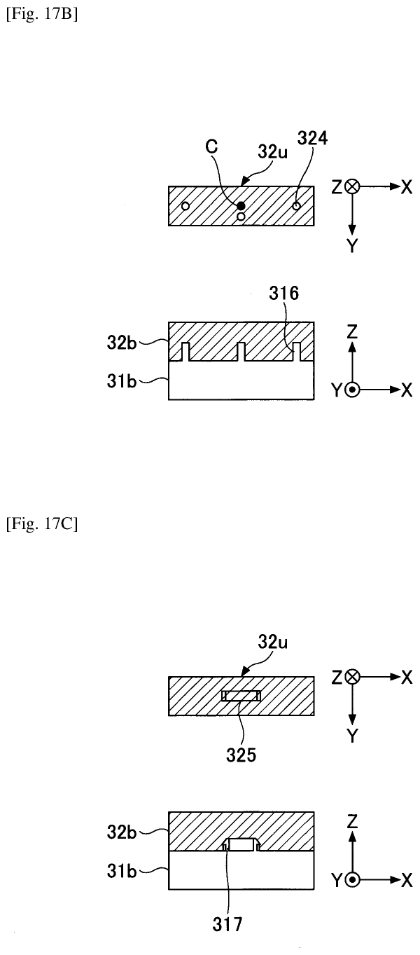

FIGS. 17A-17C are diagrams depicting variants of the structure of the part A of FIG. 16A. FIG. 17A depicts a first variant of the third embodiment, FIG. 17B depicts a second variant of the third embodiment, and FIG. 17C depicts a third variant of the third embodiment.

In the example of FIG. 17A, two knock holes 315 are provided in the first support 31b, and two knock pins 323 are provided on the second support 32b in positions corresponding to the two knock holes 315, respectively. The same advantageous effects as the advantageous effects of the third embodiment can be obtained with such a configuration. The knock holes 315 are examples of to-be-coupled units and the knock pins 323 are examples of coupling units.

In the example of FIG. 17B, three knock pins 316 are provided on the first support 31b. The three knock pins 316 are provided in asymmetric positions with respect to the center C of the face at the +Z direction side of the first support 31b. Specifically, the distance from the middle knock pin of the three knock pins to the center C is different from the distance from the left knock pin of the three knock pins to the center C. Thus, the positions of the middle knock pin and the left knock pin are asymmetric with respect to the center C.

Three knock holes 324 are provided in the second support 32b at positions corresponding to the three knock pins 316, respectively. The knock pins 316 are examples of coupling units and the knock holes 324 are examples of to-be-coupled units.

The same advantageous effects as the third embodiment can be obtained with such a configuration. At least two of the three knock pins 316 may be disposed in asymmetric positions with respect to the center C so that the first support 31b is not misoriented to be mounted to the second support 32b. The correct orientation of mounting allows the lateral side face of the ATR prism 16 supported by the second support 32b to be positioned opposite to the lateral side face faced by a living body S, thereby allowing appropriate measurement of a blood glucose level.

In the example of FIG. 17C, the first support 31b is provided with protrusions 317 with latches, and the second support 32b is provided a recess 325 with latches. When the recess 325 with the latches is aligned with respect to the protrusions 317 with the latches while the second support 32b is pressed against the first support 31b, the protrusions 317 with the latches and the recess 325 with the latches can be coupled together, and thus, the second support 32b can be mounted to the first support 31b. Latching between the protrusions 317 with the latches and the recess 325 with the latches can be released by pressing the protrusions 317 with the latches toward the inside.

The protrusions 317 with the latches are an example of a coupling unit, and the recess 325 with the latches is an example of a to-be-coupled unit. The same advantageous effects as the third embodiment can be obtained with such a configuration.

Variants will now be described for the arrangement of guiding probe light P from the QCL 110 to the ATR prism 16 and the arrangement of guiding probe light P exiting from the ATR prism 16 to the photodetector 17.

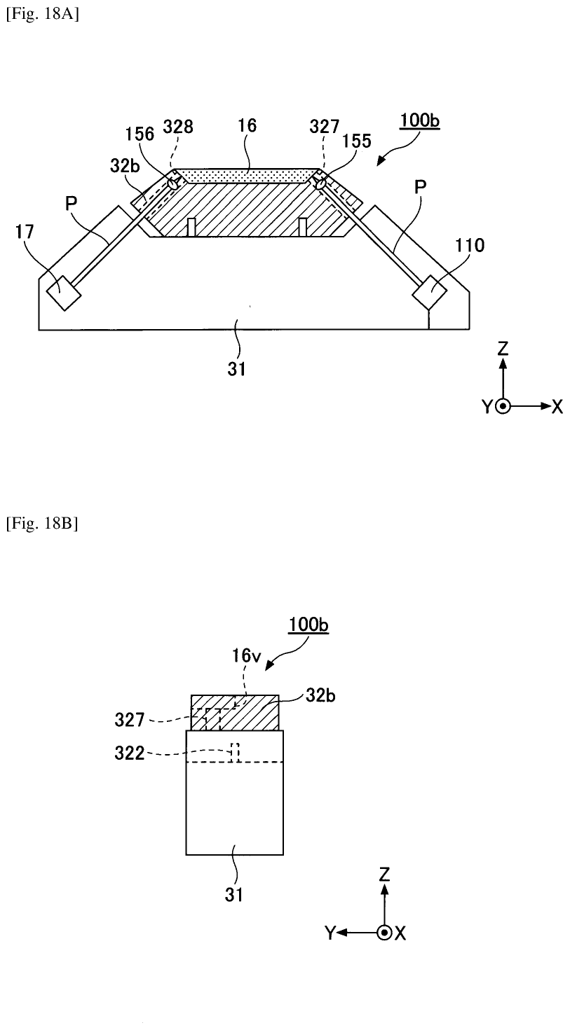

FIGS. 18A and 18B are diagrams illustrating a variant of the light guide. FIG. 18A is a front view and FIG. 18B is a side view. In the example of FIGS. 18A and 18B, a lens 155 is provided in the incidence through hole 327 in the second support 32b, and a lens 156 is provided in the outgoing through hole 328. This arrangement allows the efficiency of probe light P reaching the ATR prism 16 and the efficiency of probe light P reaching the photodetector 17 to be improved.

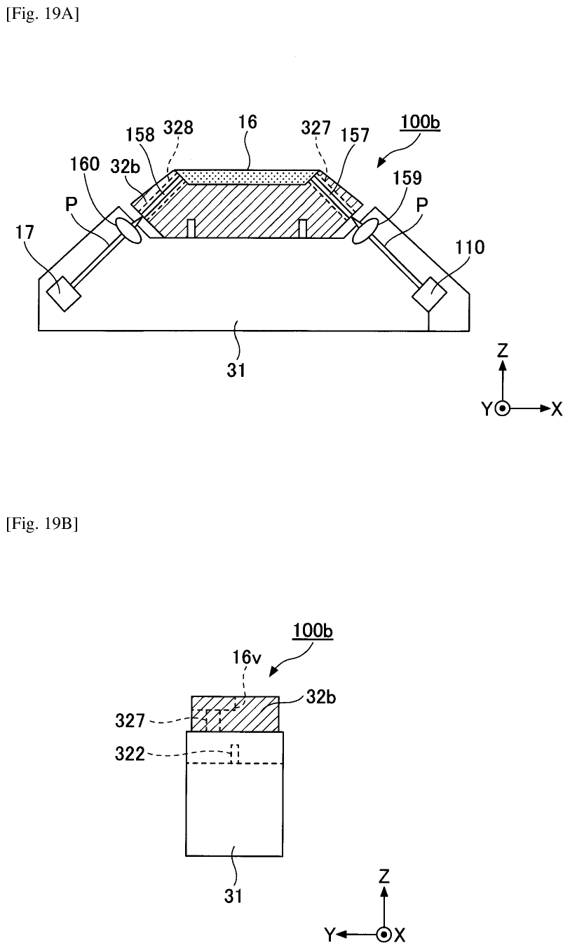

FIGS. 19A and 19B are diagrams illustrating another variant of the light guide. FIG. 19A depicts a front view and FIG. 19B depicts a side view. In the example of FIGS. 19A and 19B, a third hollow optical fiber 157 is provided in the incidence through hole 327 in the second support 32b, and a fourth hollow optical fiber 158 is provided in the outgoing through hole 328. A lens 159 is provided to the first support 31b at the side of probe light P being incident on the ATR prism 16, and a lens 160 is provided to the first support 31b at the side of probe light P exiting from the ATR prism 16. This configuration can also improve the efficiency of probe light P reaching the ATR prism 16 and the efficiency of probe light P reaching the photodetector 17.

### Fourth Embodiment

Next, the blood glucose level measuring apparatus 100c according to the fourth embodiment will be described. In this regard, the same reference numerals are given to elements identical or corresponding to elements of the first embodiment described above, and duplicate descriptions for these elements may be omitted.

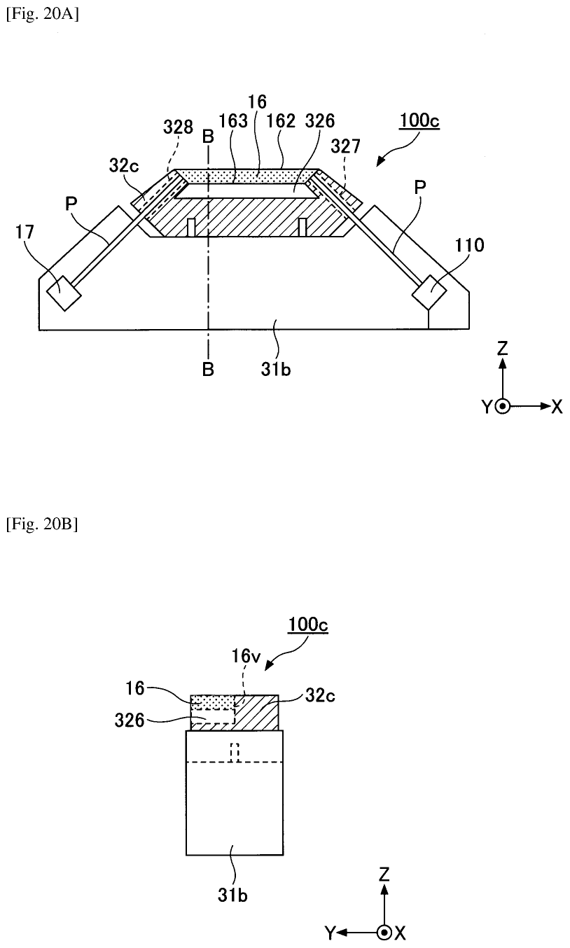

FIGS. 20A and 20B are diagrams illustrating an example of a configuration of the blood glucose level measuring apparatus 100c. FIG. 20A is a front view and FIG. 20B is a B-B cross-sectional view of FIG. 20A. As depicted in FIGS. 20A and 20B, the blood glucose level measuring apparatus 100c includes a second support 32c, which includes an open section 326.

The ATR prism 16 is fixed to the second support 32c in such a manner that the lateral side face at the −Y direction side is in contact with the +Y direction side wall of a recess 16v provided in the second support 32c. The open section 326 is a space provided below (on the −Z direction side of) the ATR prism 16 fixed to the second support 32c.

In a blood glucose level measurement in which to-be-measured person's lips are in contact with the ATR prism 16, the open section 326 allows the upper lip to contact the first total reflection face 162 and the lower lip to be inserted into the open section 326 to contact the second total reflection face 163. Accordingly, even when the block-like member is used as the second support 32c, a blood glucose level can be measured by contacting the lips on both the first total reflection face 162 and the second total reflection face 163. The other advantageous effects are the same as the corresponding advantageous effects of the second embodiment described above.

The measuring apparatuses and biological information measuring apparatuses have been described above with reference to the embodiments and variants, but the present invention is not limited to the above specifically disclosed embodiments and variants, and further variations and modifications are possible without departing from the scope of the claims.

With regard to the embodiments and variants, the example in which the functions of the absorbance obtaining unit 21, the blood glucose level obtaining unit 22, the drive control unit 23, and so forth are implemented by the single processing unit 2 has been described, but, instead, these functions may be implemented also by separate processing units, or the functions of the absorbance obtaining unit 21 and the blood glucose level obtaining unit 22 may be distributed among a plurality of processing units. In addition, the functions of the processing unit or the functions of the storage device such as the data recording unit 216 can be implemented by an external apparatus such as a cloud server.

With regard to the embodiments and variants, the example of measuring a blood glucose level as biological information has been described. However, as long as it is possible to perform measurement using the ATR method, also any other biological information can be measured with the use of any one of the embodiments and variants.

In addition, an optical element, such as a beam splitter, for branching a portion of probe light having been emitted by the light source or having exited from the hollow optical fiber, and a detection element for detecting the probe light intensity of the thus branched portion may be provided. Then, these elements may be used to implement feedback control of the driving voltage or the driving current of the light source so as to reduce the variation in the probe light intensity. This reduces the variation in output of the light source and allows for more accurate measurement of biological information.

The embodiments and variants can also be applied to a blood glucose level measuring apparatus where one wavelength of probe light is emitted by one light source for blood glucose level measurement.

The functions of each of the embodiments and variants described above may also be implemented by one or more processing circuits. The “processing circuit” used herein includes a processor programmed to perform each function by software, such as a processor implemented by electronic circuits, an application specific integrated circuit (ASIC), a digital signal processor (DSP), a field programmable gate array (FPGA), or a conventional circuit module designed to perform each function described above.

### Fifth Embodiment

In the apparatus disclosed in PTL 1, biological information is measured on the basis of a total reflection attenuation of probe light propagating inside an optical member such as an ATR prism that is in contact with a to-be-measured object.

However, in the apparatus disclosed in PTL 1, light intensity of probe light exiting from the optical member may be reduced as a result of the optical member absorbing the probe light propagating in the optical element.

It is an object of embodiments of the present invention to reduce absorption of probe light by an optical member.

In order to achieve the object, an optical member includes a total reflection member including a total reflection face configured to, in contact with an object, cause total reflection of incident probe light, and a hollow section inside the total reflection member.

In the optical member, absorption of probe light by an optical member can be reduced.

A blood glucose level measuring apparatus according to a fifth embodiment will now be described. In this regard, the same reference numerals are given to elements identical or corresponding to elements of the first embodiment described above, and duplicate descriptions for these elements may be omitted.

In the present embodiment, an optical member including a total reflection member that includes a total reflection face for, in contact with a living body S corresponding to an object, causing total reflection of incident probe light, and includes a hollow section inside is used as the total reflection member.

The term “hollow section” means a gap or a space inside the above-described total reflection member. There is a medium, with low light absorption to probe light as compared to the material of the total reflection member, inside the hollow section. An example of the medium is air, but other than air, a gas, a liquid or a solid may be inside the hollow section with less light absorption than the material of the total reflection member.

<Example Configuration of Optical Member 26>

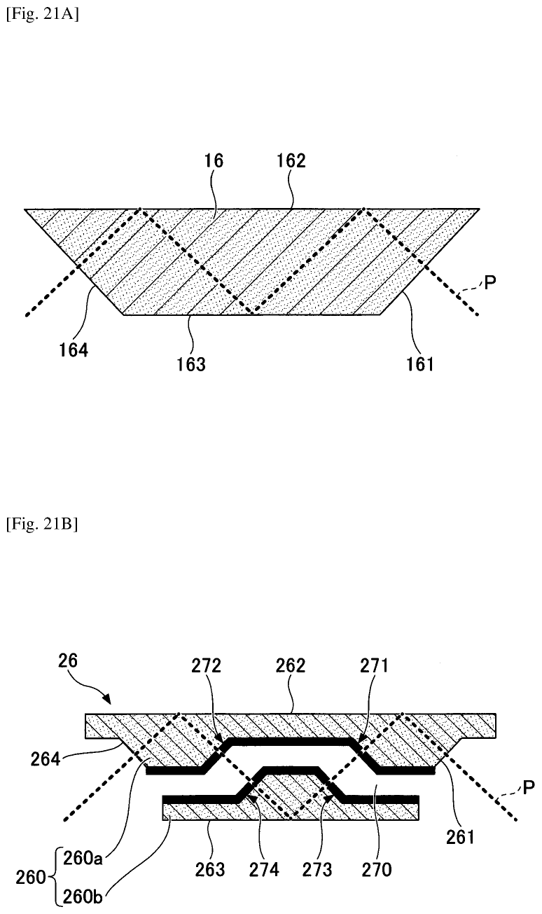

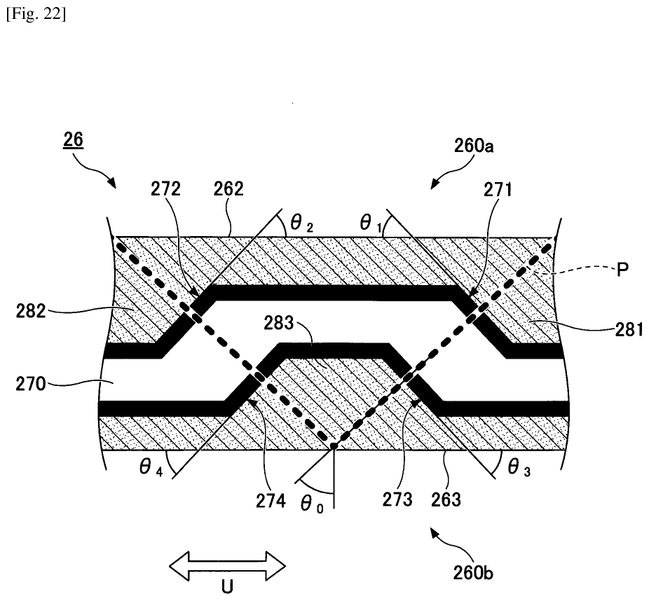

FIG. 21B is a diagram illustrating an example of a structure of an optical member 26 provided in the blood glucose level measuring apparatus according to the present embodiment. FIG. 21A depicts the ATR prism 16 as a comparative example.

In FIG. 21A, the ATR prism 16 includes the incidence face 161, the first total reflection face 162, the second total reflection face 163, and the outgoing face 164. Probe light P (broken line) emitted from the light source and incident on the ATR prism 16 at the incidence face 161 propagates through the inside of the ATR prism 16 to the first total reflection face 162 and undergoes total reflection by the first total reflection face 162. The probe light P having undergone total reflection then propagates through the inside of the ATR prism 16 to reach the second total reflection face 163 and undergoes total reflection by the second total reflection face 163. The probe light P then propagates through the inside of the ATR prism 16 to reach the first total reflection face 162, again undergoes total reflection by the first total reflection face 162, and then exits from the outgoing face 164. The light intensity of the probe light P exiting from the ATR prism 16 is detected by the photodetector 17 (see FIG. 1), and the absorbance is obtained on the basis of the detected light intensity. On the basis of the absorbance, a blood glucose level is obtained.

On the other hand, the optical member 26 according to the present embodiment includes a total reflection member 260 and a hollow section 270, as depicted in FIG. 21B. The total reflection member 260 includes a first optical block 260a and a second optical block 260b. The hollow section 270 is an air gap provided between the first optical block 260a and the second optical block 260b. The gap between the bold solid lines in FIG. 21B is the hollow section 270. The first optical block 260a is an example of a first plate-like member, and the second optical block 260b is an example of a second plate-like member.

The first optical block 260a includes an incidence face 261, a first total reflection face 262, an outgoing face 264, and inclined faces 271 and 272; and the second optical block 260b includes a second total reflection face 263 and inclined faces 273 and 274. The first optical block 260a and the second optical block 260b are each made of a silicon material that is transparent to probe light P.

The optical member 26 is positioned in place of the ATR prism 16 in FIG. 1 to function as a total reflection member that is in contact with a living body S and causes total reflection of incident probe light.

In FIG. 21B, probe light P is incident on the first optical block 260a at the incidence face 261 and propagates in the first optical block 260a to reach the first total reflection face 262. After then undergoing total reflection by the first total reflection face 262, the probe light P propagates toward the second total reflection face 263 and enters the hollow section 270 through the inclined face 271. After passing through the hollow section 270, the probe light P is incident on the second optical block 260b at the inclined face 273.

The probe light P incident on the second optical block 260b propagates in the second optical block 260b to reach the second total reflection face 263 and undergoes total reflection by the second total reflection face 263. The probe light P then propagates toward the first total reflection face 262, enters the hollow section 270 through the inclined face 274, passes through the hollow section 270, and is again incident on the first optical block 260a at the inclined face 272. Thereafter, the probe light P propagates in the first optical block 260a to reach the first total reflection face 262 and undergoes total reflection by the first total reflection face 262. After then propagating in the first optical block 260a, the probe light P exits through the outgoing face 264.

The light intensity of the probe light P thus exiting from the optical member 26 is detected by the photodetector 17 and the absorbance is obtained on the basis of the detected light intensity. On the basis of the absorbance, a blood glucose level is obtained.

FIG. 22 is an enlarged view of the inclined faces 271-274 of FIG. 21B for a more detailed description of the optical member 26.

As depicted in FIG. 22, protrusions 281 and 282 are formed from the first optical block 260a and a protrusion 283 is formed from the second optical block 260b. The protrusions 281-283 are formed to protrude alternately along the outline arrow U in the direction along each of the first and second total reflection faces 262 and 263. The inclined face 271 is formed on the protrusion 281 and the inclined face 272 is formed on the protrusion 282. The inclined faces 273 and 274 are formed on the protrusion 283.

When probe light P is incident on each of the first total reflection face 262 and the second total reflection face 263 at an angle equal to or greater than a critical angle θ_{C}, the probe light P undergoes total reflection by each of the first and second total reflection faces 262 and 263. Because the refractive index of silicon is 3.4, the critical angle θ_{C} is 39.6 degrees. Thus, probe light P undergoes total reflection when the probe light P is incident at an angle of 39.6 degrees or more on each of the first and second total reflection faces 262 and 263.

In the present embodiment, an angle of incidence of probe light P is determined in such a manner that probe light P is incident on each of the first total reflection face 262 and the second total reflection face 263 at an angle of 45 degrees with a margin to the critical angle θ_{C} in view of the spread angle of probe light P. An angle of incidence of probe light P on the first total reflection face 262 means the angle of probe light P from a normal of the first total reflection face 262; and an angle of incidence of probe light P on the second total reflection face 263 means the angle of probe light P from a normal of the second total reflection face 263.

In the present embodiment, the inclined angle θ₁ of the inclined face 271 from the first total reflection face 262 is determined to be the same as the angle of incidence θ₀, and the inclined angle θ₂ of the inclined face 272 from the first total reflection face 262 is determined to be the same as the angle of incidence θ₀. The inclined angle θ₃ of the inclined face 273 from the second total reflection face 263 is determined to be the same as the angle of incidence θ₀, and the inclined angle θ₄ of the inclined face 274 from the second total reflection face 263 is determined to be the same as the angle of incidence θ₀.

In this regard, probe light P is incident on each of the first total reflection face 262 and the second total reflection face 263 at an angle not less than the critical angle θ_{C}. That is, the inclined face 271 is inclined from the first total reflection face 262 at an angle not less than the critical angle θ_{C}, and the inclined face 272 is inclined from the second total reflection face 263 at an angle not less than the critical angle θ_{C}.

<Function and Effect of Optical Member 26>

Next, the function and effect of the optical member 26 will be described.

Zinc sulfide (ZnS) may be used as the material of the ATR prism 16 because zinc sulfide is safe for a human body and has high transmittance with respect to probe light in the mid-infrared region. However, zinc sulfide may not be superior in terms of mass-production because zinc sulfide is produced by a process such as chemical vapor deposition (CVD) or melt agglomeration, resulting in increase in the apparatus costs.

In addition, in a case of using zinc sulfide, a manufacturing process may cause a crystal lattice defect within the ATR prism 16. Such a crystal lattice defect may cause scattering of probe light P propagating in the ATR prism 16 resulting in reduction of the light intensity. As a result, the attenuation of probe light P in a living body S for measuring a blood glucose level may be unable to be accurately detected, and the accuracy of blood glucose level measurement may be reduced.

Silicon (Si) or germanium (Ge) may be considered as another material than zinc sulfide. These materials have low transmittance with respect to probe light in the mid-infrared region. Therefore, when probe light P propagates inside the ATR prism 16 made of such a material, the attenuation caused by light absorption may be increased. For example, upon propagating 10 mm inside silicon, probe light may attenuate to 10 through 20% of an incident light amount.

In addition, because the refractive index of silicon is 3.4 and is large relative to the refractive index of a living body 1.4, it is desirable that probe light P be incident on the total reflection face at an angle close to the critical angle θ_{C} in order to generate a penetrating field upon total reflection to deeply penetrate, in blood glucose level measurement. In this case, the total number of reflections occurring from probe light P being incident on the ATR prism 16 through exiting from the ATR prism 16 increases. Thus, the propagation distance of the probe light P in the ATR prism 16 increases, and the probe light P greatly attenuates depending on the amount of the propagation distance. As a result, the accuracy of blood glucose level measurement may be reduced because it may be impossible to accurately detect the attenuation of the probe light in the living body S.

According to the present embodiment, the hollow section 270 is provided inside the total reflection member 260 in the optical member 26. The hollow section 270 is filled with a medium, such as air, which absorbs less light than a silicon material. Therefore, compared to the case where probe light P propagates inside a member, such as an ATR prism, made of a silicon material, without a hollow section inside, it is possible to reduce the attenuation of the probe light P propagating in the hollow section 270. Accordingly, the attenuation of probe light passing through the optical member 26 as the total reflection member is reduced, and the attenuation of the probe light in the living body S is accurately detected, thereby ensuring the accuracy of blood glucose level measurement.

In the present embodiment, the total reflection member 260 is made of silicon. This reduces the cost of the optical member 26 and reduces the cost of the blood glucose level measuring apparatus 100 compared to the case where the total reflection member 260 is made of germanium or the like. However, the material is not limited to a silicon material, and the total reflection member 260 may be made of another material as long as the material has transparent with respect to probe light P.

In the present embodiment, portions of the hollow section 270 facing the first total reflection face 262 are provided with the inclined faces 271 and 272 that are inclined with respect to the first total reflection face 262 at the same angle as the angle of incidence θ₀ of probe light P with respect to the first total reflection face 262. In the same way, portions of the hollow section 270 facing the second total reflection face 263 are provided with the inclined faces 273 and 274 inclined with respect to the second total reflection face 263 at the same angle as the angle of incidence θ₀ of probe light P with respect to the second total reflection face 263. In other words, the inclined face 271 is inclined from the first total reflection face 262 at an angle equal to or greater than the critical angle θ_{C}, and the inclined face 272 is inclined from the second total reflection face 263 at an angle equal to or greater than the critical angle θ_{C}.

Thus, probe light P propagating in the optical member 26 can be caused to be incident perpendicular to each of the inclined faces 271-274. This reduces reflection of probe light P from the inclined faces 271-274 and reduces noise light other than probe light P undergoing total reflection inside the optical member 26, thereby improving the use efficiency of the probe light. Then, the attenuation of probe light in a living body S can be accurately detected to ensure the accuracy of blood glucose level measurement.

More suitable is antireflective coating on each of the inclined faces 271-274 preventing reflection of probe light P, to further reduce the aforementioned noise light.

In the present embodiment, the hollow section 270 is provided in a form of a gap or a space between the two optical blocks, i.e., the first optical block 260a and the second optical block 260b, but the hollow section 270 is not limited to such a structure. The two optical blocks having the hollow section 270 between these blocks may be partially connected with one another, or the hollow section 270 may be provided in a form of a gap or space inside a single optical block.

### <Variants of Optical Member 26>

The optical member 26 is not limited to having the structure depicted in FIG. 21B, and various variants are possible.

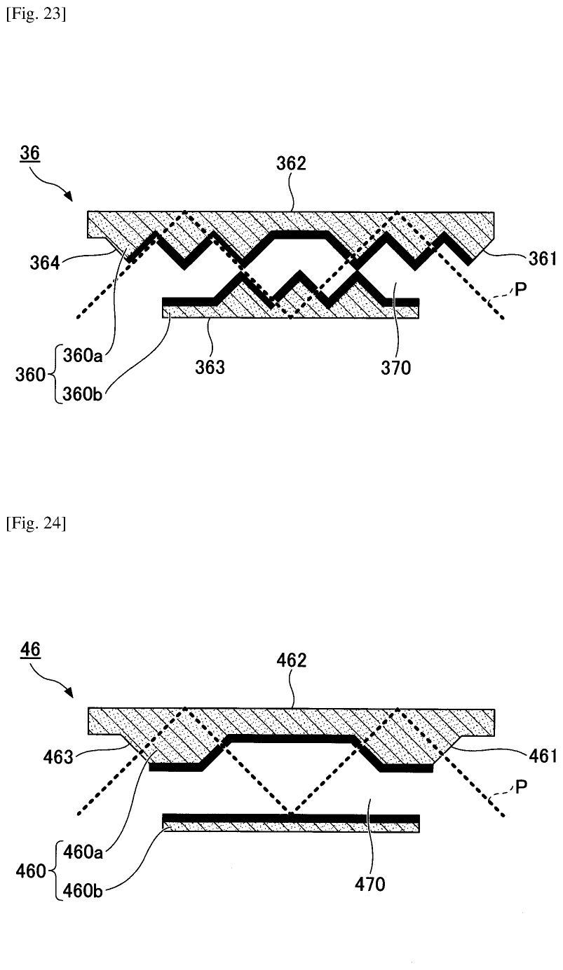

FIG. 23 is a diagram for illustrating the configuration of an optical member 36 according to a first variant of the fifth embodiment. As depicted in FIG. 23, the optical member 36 includes a total reflection member 360 and a hollow section 370. The total reflection member 360 also includes a first optical block 360a and a second optical block 360b. The hollow section 370 is an air gap provided between the first optical block 360a and the second optical block 360b. The gap between the bold solid lines in FIG. 23 is the hollow section 370.

The first optical block 360a includes an incidence face 361, a first total reflection face 362, an outgoing face 364, and 10 inclined faces; and the second optical block 360b includes a second total reflection face 363 and 6 inclined faces. Each of the first and second optical blocks 360a and 360b is made of a silicon material.

Thus, any number of inclined faces may be provided to the first optical block 360a and the second optical block 360b.

FIG. 24 is a diagram illustrating a configuration of an optical member 46 according to a second variant of the fifth embodiment. As depicted in FIG. 24, the optical member 46 includes a total reflection member 460 and a hollow section 470. The total reflection member 460 includes an optical block 460a and a mirror 460b. The hollow section 470 is an air gap provided between the optical block 460a and the mirror 460b. The gap between the bold solid lines in FIG. 24 is the hollow section 470. The mirror 460b is an example of a reflecting member.

The optical block 460a includes an incidence face 461, a total reflection face 462, an outgoing face 463, and 2 inclined faces. The optical block 460a is made of a silicon material.

Thus, not only two optical blocks are positioned opposite, but also the optical member 46 may be the total reflection member 460 including a mirror. In this case, however, the field generated along with total reflection is not generated at the mirror 460b, so blood glucose level measurement is performed on a living body S that is in contact with the total reflection face 462 of the optical block 460a.

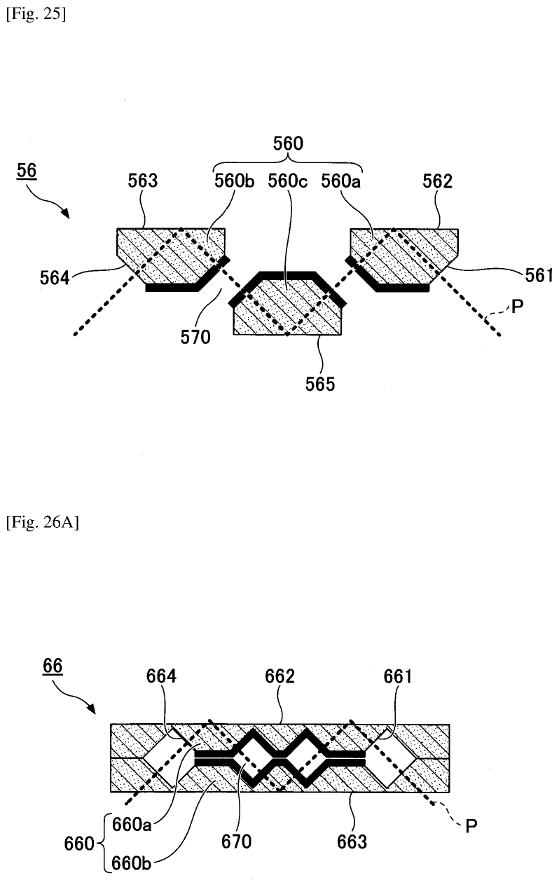

FIG. 25 is a diagram illustrating a configuration of an optical member 56 according to a third variant of the fifth embodiment. As depicted in FIG. 25, the optical member 56 includes a total reflection member 560 and a hollow section 570. The total reflection member 560 includes a first optical block 560a, a second optical block 560b, and a third optical block 560c. The hollow section 570 includes air gaps provided between the first optical block 560a, the second optical block 560b, and the third optical block 560c. The gaps between the bold solid lines in FIG. 25 are the hollow section 570.

The first optical block 560a includes an incidence face 561 and a first total reflection face 562, the second optical block 560b includes a second total reflection face 563 and an outgoing face 564, and the third optical block 560c includes a third total reflection face 565. The first optical block 560a, the second optical block 560b, and the third optical block 560c each is made of a silicon material. The first optical block 560a, the second optical block 560b, and the third optical block 560c are examples of a plurality of plate-like members.

Thus, not only two optical blocks are positioned opposite, but also the optical member 56 may be the total reflection member 560 including three or more optical blocks in combination.

Each of the optical members 36, 46, and 56 described above is positioned in place of the ATR prism 16 of FIG. 1 to serve as a total reflection member which, in contact with a living body S, causes total reflection of incident probe light.

<Example of Manufacturing Optical Member According to Present Embodiment>

A method of manufacturing an optical member according to the present embodiment will now be described.

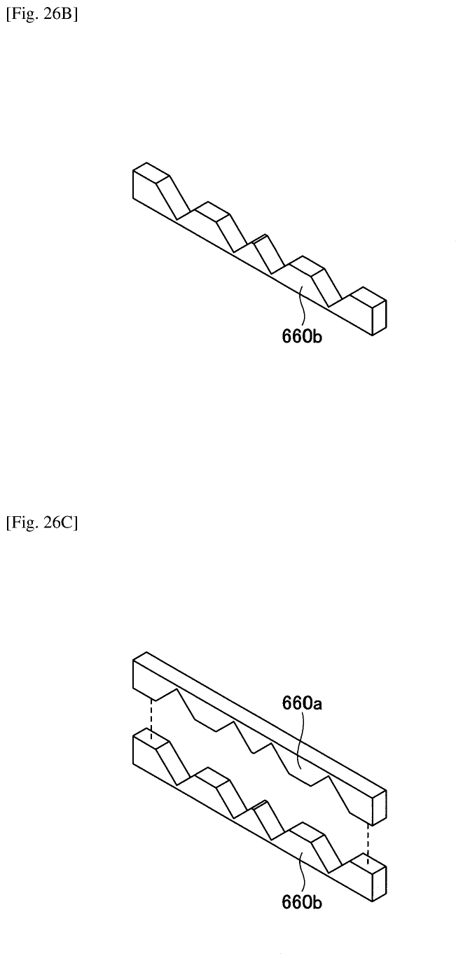

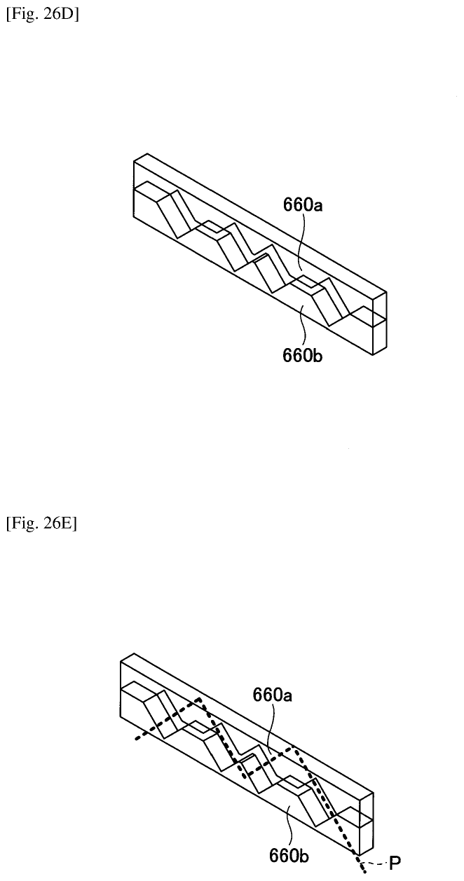

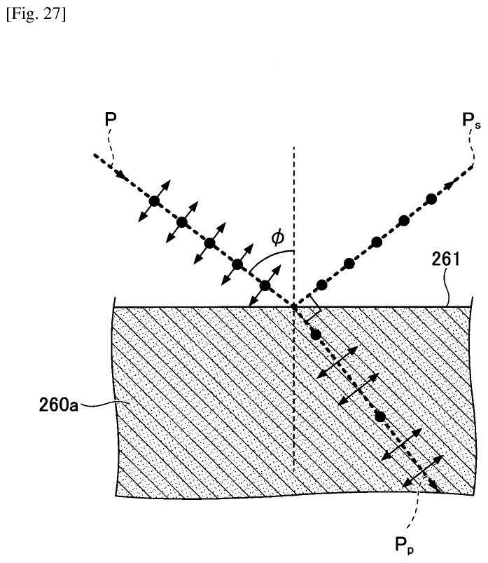

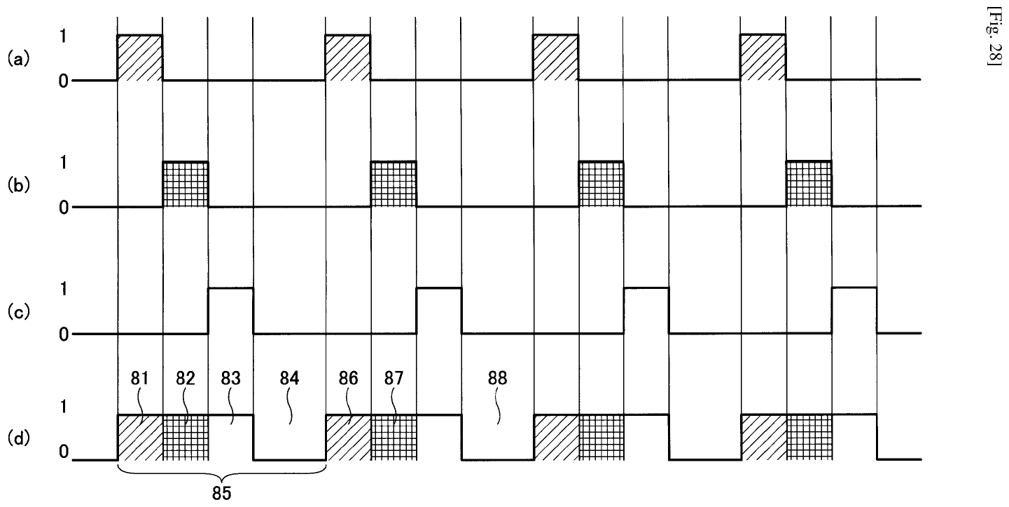

FIGS. 26A-26E are diagrams illustrating an example of a method of manufacturing the optical member 66: FIG. 26A illustrates a configuration of the optical member 66, and FIG. 26B-26E depict respective states of the optical member 66 in the manufacturing process. FIG. 26B depicts the second optical block 660b, FIG. 26C depicts the first optical block 660a and the second optical block 660b before being jointed, FIG. 26D depicts the first optical block 660a and the second optical block 660b after being jointed, and FIG. 26E depicts a propagation of probe light P in the optical member 66.

In manufacturing the optical member 66, the second optical block 660b is manufactured as depicted in FIG. 26B by first forming a groove in a silicon wafer and cutting out a block for a predetermined size through anisotropic etching. Similarly, the first optical block 660a is manufactured by forming a groove in the silicon wafer and cutting out a block for a predetermined size through anisotropic etching. Then, as depicted in FIGS. 26C and 26D, edges of the first optical block 660a and the second optical block 660b are used to adjust the respective positions with each other and the two blocks are bonded together. Thus, the optical member 66 can be manufactured.

However, manufacturing of the first and second optical blocks 660a and 660b is not limited to the above-described way of using anisotropic etching, and any other machining method such as optical or thermal imprinting, injection molding, or cutting may be used. Desirably, a specific machining method is selected depending on the materials of the first optical block 660a and the second optical block 660b.

### Sixth Embodiment

A sixth embodiment of the present invention will now be described. In this regard, the same reference numerals are given to elements identical or corresponding to elements of the first embodiment described above, and duplicate descriptions for these elements may be omitted.

With regard to the fifth embodiment described above, the example of providing an antireflective coating on the inclined faces included in the optical member 26 has been described, but embodiments are not limited to such a configuration. Instead of providing an antireflective coating, or in addition to providing an antireflective coating, the polarization state of probe light P may be p-polarized and caused to be incident on the incidence face 261, outgoing face 264, and inclined faces 271-274, respectively, of the optical member 26. This can reduce reflection of the probe light P from each of the incidence face 261, outgoing face 264, and inclined faces 271-274 as compared to when probe light P includes a component of an s-polarized light state.

It is further desirable that p-polarized probe light P be incident at an angle corresponding to a Brewster angle on each of the incidence face 261, outgoing face 264, and inclined faces 271-274. A Brewster angle is an angle of incidence at which the reflectivity of p-polarized light is zero at an interface between materials having different refractive indexes. An angle corresponding to a Brewster angle refers to each of both an angle that is the same as a Brewster angle and an angle that differs from a Brewster angle by a generally acceptable degree of machining or manufacturing error.

FIG. 27 is a diagram illustrating a state in which probe light P is incident on the incidence face 261 at a Brewster angle φ. When being incident on the incidence face 261 at a Brewster angle φ, the p-polarized component P_{P} of probe light P is incident on the first optical block 260a without reflection, and only the s-polarized component P_{S} is reflected. Therefore, it is possible to eliminate reflection to the utmost, by generating probe light P whose polarization state is a p-polarized state using a polarizing device or the like and causing the probe light to be incident on the incidence face 261 at an angle corresponding to a Brewster angle.

This reduces noise light other than light undergoing total reflection included in probe light P inside the optical member 26, thereby improving the use efficiency of probe light. Then, attenuation of probe light by a living body S can be accurately detected to ensure the accuracy of blood glucose level measurement.

While the fifth and sixth embodiments of the first embodiment have been described above, further variations and modifications are possible.

With regard to the fifth and sixth embodiments described above, the example in which the functions of the absorbance obtaining unit 21, the blood glucose level obtaining unit 22, the drive control unit 23, and so forth are implemented by the single processing unit 2 has been described, but, instead, these functions may be implemented also by separate processing units, or the functions of the absorbance obtaining unit 21 and the blood glucose level obtaining unit 22 may be distributed among a plurality of processing units. In addition, the functions of the processing unit or the functions of the storage device such as the data recording unit 216 can be implemented by an external apparatus such as a cloud server.

In addition, the example in which the first light source 111, second light source 112, and third light source 113 are used as the plurality of light sources, each of which emits light of a different wavelength in the mid-infrared region, has been described, but, instead, a single light source may emit light of multiple wavelengths.

Also, although the examples using the quantum cascade lasers have been depicted as the light sources, the light sources are not limited to quantum cascade lasers. Light sources other than lasers such as infrared lamps, light emitting diodes (LED), super luminescent diodes (SLD) may be used instead. In such a case, it may be desirable to use a wavelength filter for obtaining only a desired wavelength and to cause probe light to be incident on the total reflection member, such as the ATR prism 16, through the filter. Alternatively, the photodetector 17 may desirably receive probe light through a wavelength filter.

With regard to the embodiments and variants described above, the examples of measuring blood glucose levels as biological information have been described. However, as long as it is possible to measure using the ATR method, also any other biological information can be measured with the use of any one of the embodiments and variants.

In addition, an optical element, such as a beam splitter, for branching a portion of probe light after the probe light is emitted by the light source or exits from the hollow optical fiber, as well as a detection element for detecting the probe light intensity of the thus branched portion may be provided to implement feedback control of a driving voltage or a driving current of the light source so as to reduce the variation in the probe light intensity. This reduces the variation in output of the light source and allows for more accurate measurement of biological information.

The embodiment and variants can also be applied to blood glucose level measuring apparatuses where one wavelength of probe light is emitted by one light source for blood glucose level measurement.

With regard to the embodiments and variants, examples using a plurality of light sources have been described, but, instead, the embodiments and variants can also be applied to blood glucose level measuring apparatuses each including one light source which emits first through third probe lights of different wavelengths from the one light source. In that case, the blood glucose level measuring apparatus need not include the first shutter 121, second shutter 122, third shutter 123, first half mirror 131, and second half mirror 132 because switching among first through third probe lights to be incident on the ATR prism 16 is not needed.

The embodiments and variants can also be applied to blood glucose level measuring apparatuses including one light source which emits one wavelength of probe light.

The functions of each of the embodiments and variants described above may also be implemented by one or more processing circuits. The “processing circuit” used herein includes a processor programmed to perform each function by software, such as a processor implemented by electronic circuits, an application specific integrated circuit (ASIC), a digital signal processor (DSP), a field programmable gate array (FPGA), or a conventional circuit module designed to perform each function as described above.

### Seventh Through Twelfth Embodiments

Next, sixth through twelfth embodiments of the present invention will be described.

PTL1 discloses a technology of measuring absorbance with respect to light, which has wavelengths between a plurality of absorption peaks in glucose, by using a total reflection member that causes total reflection of probe light in a specific wavelength region, such as a mid-infrared region, in a state of being in contact with a to-be-measured object.

However, in the technology of PTL 1, it may be impossible to accurately measure absorbance of a to-be-measured object because of a variation in the contact pressure of the object to a total reflection member.

It is an object of embodiments of the present invention to accurately measure absorbance with respect to light in a specific wavelength region.

In order to achieve the object, an absorbance measuring apparatus includes a light source configured to emit probe light in a specific wavelength region; a total reflection member configured to, in contact with a to-be-measured object, causes total reflection of the probe light that is incident; a pressure detector configured to detect a pressure of the to-be-measured object to the total reflection member; a light intensity detector configured to detect light intensity of the probe light exiting from the total reflection member; and an absorbance output unit configured to output absorbance of the probe light obtained on the basis of the light intensity and the pressure.

In the absorbance measuring apparatus, absorbance of light in a specific wavelength region can be accurately measured.

In a technology in which absorbance is measured according to the ATR method using probe light of a specific wavelength region, such as a mid-infrared region, a measurement value may vary due to adhesion of a contact surface to a to-be-measured object. In this regard, PTL 3 discloses a technology in which a groove for lifting the to-be-measured object is provided at a to-be-measured object contact surface of a total reflection member such as an ATR prism.

However, in the technology of PTL 3, it may be impossible to accurately measure absorbance due to a variation in the contact area between the total reflection member and the to-be-measured object.

It is an object of embodiments of the present invention to accurately measure absorbance with respect to light in a specific wavelength region even in the above-described situation.

In order to achieve the object, an absorbance measuring apparatus includes a light source for emitting probe light in a specific wavelength region; a total reflection member having an incidence face on which the probe light emitted from the light source is incident, a total reflection face for, in contact with a to-be-measured object, causing total reflection of the probe light, and an outgoing face from which the probe light that undergoes total reflection by the total reflection face exits; a light intensity detector for detecting light intensity of the probe light exiting from the outgoing face; and an absorbance output unit for outputting absorbance with respect to the probe light obtained on the basis of the detected light intensity. The total reflection member includes an area defining section for defining a measurement sensitivity area for measuring absorbance at the total reflection face.

In such an absorbance measuring apparatus, absorbance with respect to light in a specific wavelength region can be accurately measured.

Hereinafter, the seventh through twelfth embodiments of the present invention will be described with reference to the drawings. In each drawing, the identical elements are indicated by the same reference numerals, and duplicate descriptions may be omitted.

### Seventh Embodiment

First, a blood glucose level measuring apparatus 100 according to the seventh embodiment will be described. The seventh embodiment is similar to the first embodiment described above with reference to FIGS. 1-14B. Therefore, mainly, the points different from the first embodiment will be described, and duplicate description may be omitted.

In the present embodiment, a plurality of probe lights having different wavelengths in the mid-infrared region are incident on a total reflection member provided in contact with a living body, and absorbance with respect to each of the plurality of probe lights is measured on the basis of the ATR method.

A light intensity detector detects light intensity of probe light exiting from the total reflection member, and incidence of probe light on the total reflection member is controlled in such a manner that at least a non-incidence period during which all of the plurality of probe lights are not incident on the total reflection member is provided. Then, absorbance data with respect to light in the mid-infrared region is obtained on the basis of detection values obtained by the light intensity detector when probe light is incident on the total reflecting member and detection values obtained by the light intensity detector when all of the plurality of probe lights are not incident on the total reflecting member. This reduces the influence of the ambient environment of the blood glucose level measuring apparatus on the measurement and a temperature change of the living body, and thus, absorbance is accurately measured.

### <Overall Configuration Example of Blood Glucose Level Measuring Apparatus 100>

The overall configuration of the blood glucose level measuring apparatus 100 according to the seventh embodiment is the same as the overall configuration of the first embodiment described above with reference to FIG. 1.

The measuring unit 1 of the blood glucose level measuring apparatus 100 of the present embodiment is an optical head for performing an ATR method and outputs a detection signal of probe light attenuated in a living body to the processing unit 2. The processing unit 2 obtains absorbance data through calculation on the basis of the detection signal, obtains a blood glucose level through calculation on the basis of absorbance data, and outputs the blood glucose level.

<Function and Configuration of ATR Prism 16>

The function and configuration of the ATR prism 16 is the same as the function and configuration of the ATR prism 16 of the first embodiment described above with reference to FIGS. 2-4.

<Configuration of Processing Unit 2>

The configuration of the processing unit 2 is the same as the configuration of the processing unit 2 of the first embodiment described above with reference to FIGS. 5 and 6.

<Example of Operation of Blood Glucose Level Measuring Apparatus 100>

Next, operation of the blood glucose level measuring apparatus 100 according to the seventh embodiment will be described.

(Example of Probe Light Switching Operation)

An example of a probe light switching operation is the same as an example of a probe light switching operation of the first embodiment described above with reference to FIGS. 7A-7C.

(Example of Probe Light Switching Timing)

FIG. 28 is a timing chart illustrating an example of switching timing of the first through third probe lights. FIG. 28, (a) depicts a state of the first shutter 121, (b) depicts a state of the second shutter 122, (c) depicts a state of the third shutter 123, and (d) depicts an output signal of the photodetector 17. In each figure, when the signal level is 0, the shutter is closed, and when the signal level is 1, the shutter is opened. In addition, the signal represented by diagonal hatching is for first probe light, the signal represented by lattice hatching is for second probe light, and the signal represented without hatching is for third probe light.

In FIG. 28, (a), the shutter control unit 214 opens the first shutter 121, and closes the second shutter 122 and the third shutter 123. As depicted in FIG. 28, (d), for a period 81 during which the first shutter 121 is open, the photodetector 17 outputs a detection signal when first probe light is incident on the ATR prism 16.

Then, after a predetermined time has elapsed, the shutter control unit 214 opens the second shutter 122 at a time of closing the first shutter 121 (FIG. 28, (b)). As depicted in FIG. 28, (d), during a period 82 during which the second shutter 122 is open, the photodetector 17 outputs a detection signal when second probe light is incident on the ATR prism 16.

Then, after a predetermined time has elapsed, the shutter control unit 214 opens the third shutter 123 at a time of closing the second shutter 122 (FIG. 28, (c)). As depicted in FIG. 28, (d), during a period 83 during which the third shutter 123 is open, the photodetector 17 outputs a detection signal when third probe light is incident on the ATR prism 16.

Then, when the shutter control unit 214 closes the third shutter 123 after a predetermined time has elapsed, all of the first shutter 121, the second shutter 122, and the third shutter 123 are in the closed states. The photodetector 17 outputs a detection signal in a state where all of first through third probe lights are not incident on the ATR prism 16 as in a non-incidence period 84 depicted in FIG. 28, (d).

Then, after a predetermined period of time has elapsed, the shutter control unit 214 opens the first shutter 121, the second shutter 122, and the third shutter 123 sequentially each for a predetermined period of time, and then, closes all of the shutters. Then, such operations are repeated.

Thus, the shutter control unit 214 as an incidence control unit can control incidence of first through third probe lights on the ATR prism 16 such that at least a non-incidence period 84 in which all of first through third probe lights are not incident on the ATR prism 16 is provided.

The cycle 85 of FIG. 28, (d) represents one cycle of control operation by the shutter control unit 214. Each single cycle includes a period during which first through third probe lights are sequentially incident on the ATR prism 16 and a period during which all of first through third probe lights are not incident on the ATR prism 16, as depicted in FIG. 28, (d).

During each period in a cycle 85, the photodetector 17 outputs a light intensity detection signal to the data recording unit 216 via the data obtaining unit 215. The data recording unit 216 separately stores a first detection value on the basis of a detection signal of first probe light, a second detection value on the basis of a detection signal of second probe light, a third detection value on the basis of a detection signal of third probe light, and a fourth detection value on the basis of a detection signal at a non-incidence period, in a distinguishable manner.

A function of a fourth detection value on the basis of a detection signal at a non-incidence period will now be described. A detection signal of the photodetector 17 includes light intensity of background light around the blood glucose level measuring apparatus 100 as a bias signal, and, in the mid-infrared region, the photodetector 17 also detects radiation (heat rays) due to heat as light intensity, so that the bias signal includes a large amount of light intensity of heat rays.

When the bias signal level changes due to a change in background light intensity, a change in temperature around the blood glucose level measuring apparatus, or the like, absorbance data obtained on the basis of a detection signal of the photodetector 17 changes, resulting in a measurement error. In particular, the temperature varies from hour to hour depending on the ambient environment of the blood glucose level measuring apparatus, heat emitted by a living body, heat emitted by the light sources and the photodetector, and the like. The level of the bias signal varies accordingly, and thus, the accuracy of measurement may degrade.

A detection signal of the photodetector 17 at a non-incidence period 84 in FIG. 28 represents such a bias signal that does not include first through third probe light intensities. Therefore, according to the present embodiment, the bias signal components included in first through third detection values detected based on first through third probe lights, respectively, are removed by subtracting a fourth detection value of a non-incidence period 84 from each of the first through third detection values. This allows absorbance data to be obtained with reduced influence of ambient environment, a temperature change of a living body, and so forth, using detection values of first through third probe lights with bias signal components removed.

If a time difference between a period when probe light is detected and a non-incidence period increases, the change in the bias signal level due to the temperature and the like due to the time difference may increase, and the influence of the bias signal may not be sufficiently removed. Therefore, in the present embodiment, the influence of the bias signal is removed by using the detection value at the non-incidence period nearest to the period when the detection value of probe light is obtained.

For example, in FIG. 28, the first detection value at the first probe light detection period 86 is corrected using the fourth detection value at the nearest non-incidence period 84, not the non-incidence period 88 after the period 86. The second detection value at the second probe light detection period 87 is corrected using the fourth detection value at the nearest non-incidence period 84 or non-incidence period 88. Thus, an influence of a temporal change in temperature or the like is more desirably reduced.

The above-mentioned period 86 is an example of a first incidence period and the period 87 is an example of a second incidence period. These periods 86, 87, and 88 are periods included in one cycle. Thus, the absorbance output unit 217 can output absorbance data in which an influence of a bias signal is removed.

(Example of Operation of Blood Glucose Level Measuring Apparatus 100)

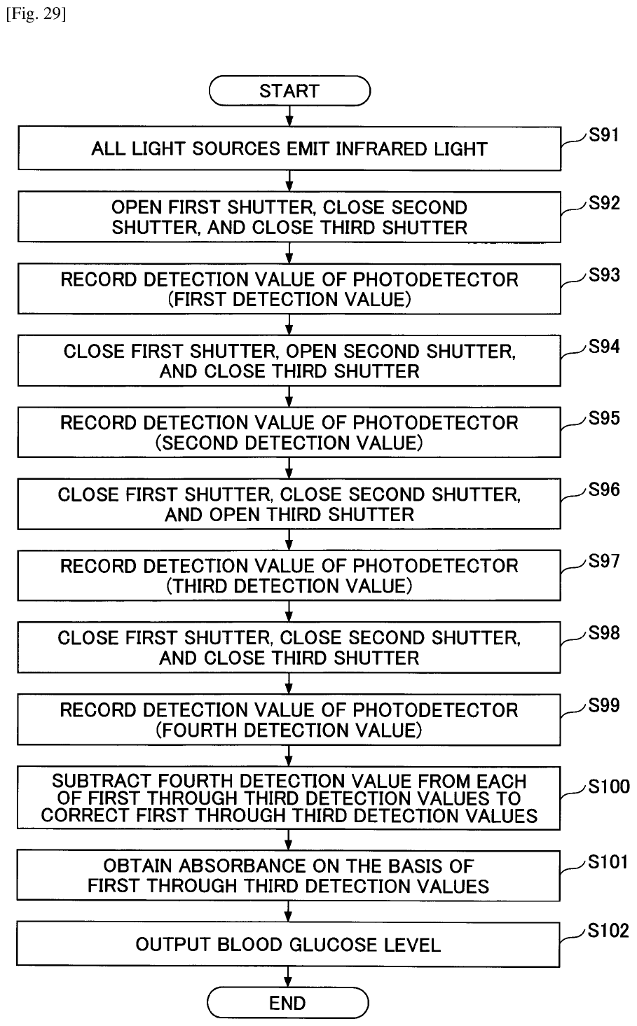

FIG. 29 is a flowchart illustrating an example of an operation of the blood glucose level measuring apparatus 100 according to the seventh embodiment.

First, in step S91, in response to a control signal of the light source control unit 212, all of the first light source 111, the second light source 112, and the third light source 113 emit infrared light. However, in this initial state, the first shutter 121, the second shutter 122, and the third shutter 123 are all closed.

Subsequently, in step S92, the shutter control unit 214 opens the first shutter 121 and keeps the closed states of the second shutter 122 and the third shutter 123.

Subsequently, in step S93, the data recording unit 216 stores a detection value (a first detection value) of the photodetector 17 obtained by the data obtaining unit 215.

Subsequently, in step S94, the shutter control unit 214 opens the second shutter 122, closes the first shutter 121, and keeps the closed state of the third shutter 123.

Subsequently, in step S95, the data recording unit 216 stores a detection value (a second detection value) of the photodetector 17 obtained by the data obtaining unit 215.

Subsequently, in step S96, the shutter control unit 214 opens the third shutter 123, keeps the closed state of the first shutter 121, and closes the second shutter 122.

Subsequently, in step S97, the data recording unit 216 stores a detection value (a third detection value) of the photodetector 17 obtained by the data obtaining unit 215.

Subsequently, in step S98, the shutter control unit 214 keeps the closed states of the first shutter 121 and the second shutter 122, and closes the third shutter 123.

Subsequently, in step S99, the data recording unit 216 stores a detection value (a fourth detection value) of the photodetector 17 obtained by the data obtaining unit 215.

Subsequently, in step 5100, the absorbance output unit 217 corrects each of the first through third detection values read from the data recording unit 216 by subtracting the fourth detection value at the period nearest to the period at which each of the detection values has been obtained.

Subsequently, in step 5101, the absorbance output unit 217 obtains absorbance data of the first through third probe lights on the basis of the first through third detection values after the correction and outputs the absorbance data to the biological information output unit 221.

Subsequently, in step 5102, the biological information output unit 221 performs a predetermined calculation process on the basis of the absorbance data of the first through third probe lights and obtains blood glucose level data. The obtained blood glucose level data is output to the display 506 (see FIG. 5) for display.

Thus, the blood glucose level measuring apparatus 100 according to the seventh embodiment can obtain and output blood glucose level data.

<Advantageous Effect of Seventh Embodiment>

The mid-infrared region is the fingerprint region where glucose absorption is high, and is advantageous in that it is possible to improve measurement sensitivity in comparison to the near-infrared region. However, because the mid-infrared region includes a wavelength region of a radiation spectrum of an object with respect to room temperature, a detection signal of the photodetector varies from time to time depending on the ambient environment of the blood glucose level measuring apparatus, the heat emitted by a living body, and the heat emitted by the light source and the photodetector used in the blood glucose level measuring apparatus. In particular, in a method of contacting a living body with a total reflection member, such as an ATR prism, heat transfer from the living body may cause the temperature of the total reflection member or the living body to change in a short time, making it impossible to accurately measure the absorbance.

Also, the accuracy of measuring a blood glucose level may be reduced when light of a single wavelength or a narrow band of wavelengths near a single wavelength is used (see, e.g., Kasahara. R, Kino. S, Soyama. S, Matsuura. Y, “Noninvasive glucose monitoring using mid-infrared absorptive spectroscopy on the basis of a few wavenumbers,” Biomedical Optics expression, 2018, 9 (1), pages 289-302).

In the present embodiment, the ATR prism 16 provided in contact with a living body S is radiated with first through third probe lights having different wavelengths in the mid-infrared region, and absorbance with respect to each of the first through third probe lights is measured according to the ATR method.

The photodetector 17 is provided for detecting light intensities of first through third probe lights exiting from the ATR prism 16, and incidence of the first through third probe lights on the ATR prism 16 is controlled in such a manner that at least a non-incidence period during which all of the first through third probe lights are not incident on the ATR prism 16 is provided. Then, absorbance data with respect to light in the mid-infrared region is obtained on the basis of first through third detection values of the photodetector 17 obtained when the respective first through third probe lights are incident on the ATR prism 16 and the fourth detection value of the photodetector 17 obtained during the non-incidence period .

Because a fourth detection value is based on a bias signal caused by the ambient environment of the blood glucose level measuring apparatus or the heat of a living body S, the influence of measurement of the surrounding environment of the blood glucose level measuring apparatus or the temperature change of the living body can be reduced by subtracting the fourth detectable value from each of the first through third detectable values for correction of the first through third detection values. This allows accurate measurement of absorbance.

The above-described correction can be implemented by any correction process with the use of first through third detection values and a fourth detection value, but the correction can be more easily implemented through an operation of subtracting a fourth detection value from each of first through third detection values.

In the present embodiment, the shutter control unit 214 as the incident control unit perform periodic control to cause one cycle to include a period during which first through third probe lights are incident on the ATR prism 16 one by one in sequence and a non-incidence period during which all of first through third probe lights are not incident on the ATR prism 16.

Thus, absorbance data corrected on the basis of first through third detection values and a fourth detection value can be obtained repeatedly, and absorbance that changes in time can be accurately measured at each time point.

If a time difference between each of periods at which first through third detection values are obtained and a corresponding non-incidence period at which a fourth detection value is obtained is large, a variation in temperature or the like due to the time difference may be large, and the influence of the bias signal may not be removed sufficiently.

Therefore, in the present embodiment, the shutter control unit 214 as the incidence control unit periodically controls a period 86 (first period of incidence) during which first probe light among first through third probe lights are incident on the ATR prism 16, a period 87 (second period of incidence) during which second probe light among first through third probe lights is incident on the ATR prism 16, and a non-incidence period 88 to be included in one cycle.

The absorbance output unit 217 obtains first absorbance data on the basis of a first detection value at the period 86 and a fourth detection value in the period 84 nearest to the period 86, and also, obtains second absorbance data on the basis of a second detection value at the period 87 and a fourth detection value at the nearest non-incidence period 84 or non-incidence period 88. The first absorbance data is an example of first absorbance, and the second absorbance data is an example of second absorbance.

Thus, a detection value on the basis of a bias signal nearest to the time when a detection value of probe light is obtained can be obtained, and absorbance can be more accurately measured by minimizing the influence of a temperature change in a living body or the like.

In the seventh embodiment, an example in which the first shutter 121, the second shutter 122, and the third shutter 123, which are electromagnetic shutters, are controlled to switch incident probe light on the ATR prism 16 has been described, but incident light switching control is not limited to such a control manner. Incidence of probe light on the ATR prism 16 may be instead switched between turning on (emission) and turning off (not emission) of a plurality of light sources. In addition, a single light source that emits light of multiple wavelengths may be instead used to switch incident light by turning on and turning off with respect to each wavelength.

With regard to the seventh embodiment, the example where the first half mirror and the second half mirror are used as elements each transmitting a portion of probe light and reflecting the rest. However, instead, also a beam splitter, a polarizing beam splitter, or the like may be used for the same purpose.

In addition, high refractive index materials, such as germanium, that transmit probe light have high surface reflectivity due to material characteristics. For example, when light (s-polarized) polarized in the vertical direction with respect to the plane direction of a substrate enters the substrate at an angle of incidence of 45 degrees, the ratio of transmission to reflection becomes approximately 1:1. This can be used to install a germanium plate at an angle of incidence of 45 degrees to replace the half mirror. In the same way, a back side has a 50% reflective component, so an anti-reflection coating is applied to the back side.

<Variants of Seventh Embodiment>

Hereinafter, variations will be described, because there are variants to elements of the seventh embodiment.

(Timing of Non-Incidence Period)

First, with regard to the seventh embodiment described above, an example in which first through third probe lights are sequentially incident on the ATR prism 16 for corresponding periods of time, followed by a non-incidence period has been described.

Alternatively, a non-incidence period may be provided after a period during which first probe light is incident on the ATR prism 16; a non-incidence period may be provided after a period during which second probe light is incident on the ATR prism 16; and a non-incidence period may be provided after a period during which third probe light is incident on the ATR prism 16. In this manner, it is easier to obtain a detection value on the basis of a bias signal at the nearest period, and it is possible to more accurately remove the influence of a temperature change at a living body or the like.

(Control of Influence of Linearity Error of Photodetector 17)

Variants of the seventh embodiment concerning control of influence of linearity error of the photodetector 17 are almost the same as variants concerning control of influence of linearity error of the photodetector 17 of the first embodiment described above with reference to FIGS. 9A and 9B. Therefore, mainly, different points will now be described. As described above, the light intensity of probe light in the case of FIG. 9B changes at a cycle shorter than the probe light switching control cycle of the shutter control unit 214, i.e., according to the seventh embodiment, for example, the cycle from Step S92 to S94 in FIG. 29. The probe light switching control period of the shutter control unit 214 corresponds to “a control period of an incidence control unit.”

### (Detection of Probe Light by Image Sensor)

Variants of the seventh embodiment concerning detection of probe light by an image sensor are the same as variants concerning detection of probe light by an image sensor of the first embodiment described above with reference to FIGS. 10A and 10B.

### (Incidence Face of Total Reflection Member)

Variants of the incidence face 161 of the ATR prism 16 are the same as variants of the incidence face 161 of the ATR prism 16 of the first embodiment described above with reference to FIGS. 11A-11E.

(Support of Light Guide and Total Reflection Member)

Variants of the seventh embodiment concerning a support of the light guide and the total reflection member are the same as variants concerning a support of the light guide and the total reflection member of the first embodiment described above with reference to FIGS. 12A-13.

(Detection and Indication of Contact State)

When the ATR prism 16 comes into contact with a body part (a lip or the like) of a to-be-measured person not included in the view of the to-be-measured person for whom a blood glucose level is measured, the contact state between the body part and the ATR prism 16 cannot be visually perceived by the person. Therefore, the contact state may change for each measurement occasion and a measurement variation may increase.

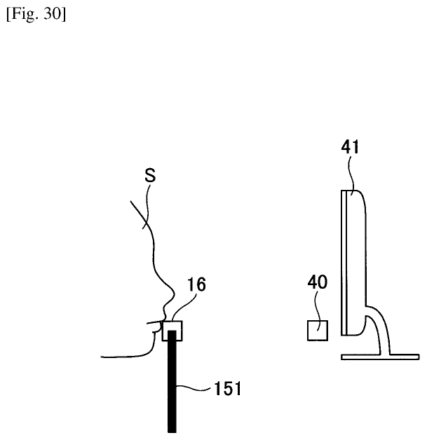

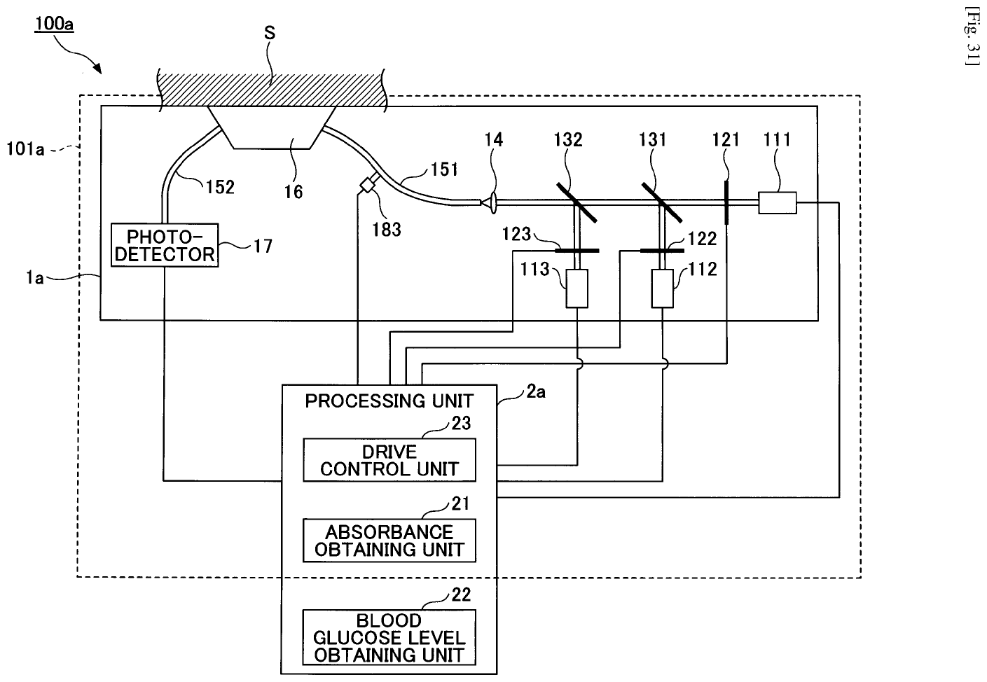

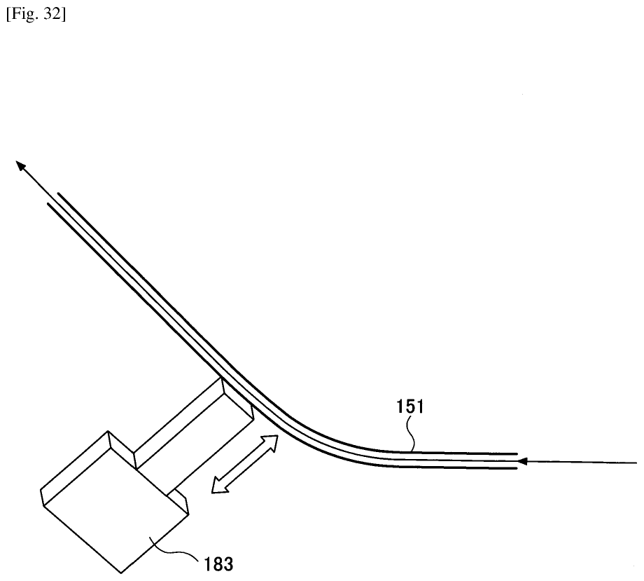

In this regard, as depicted in FIG. 30, a camera 40 for capturing an image of the contact position between the lip of the person, i.e., a living body S and the ATR prism 16, as well as a display unit 41 such as a liquid crystal display for displaying the image captured by the camera 40, may be added to the blood glucose level measuring apparatus 100.

By adjusting the contact state between the lip and the ATR prism 16 while the person visually sees the image displayed on the display unit 41, the reproducibility of the contact state can be improved and a measurement variation can be reduced.

### Eighth Embodiment

Next, a blood glucose level measuring apparatus 100a according to an eighth embodiment of the present invention will be described.

The eighth embodiment is similar to the first embodiment described above with reference to FIGS. 1-14B. Therefore, mainly, the points different from the first embodiment will be described, and duplicate description may be omitted.

In the present embodiment, a first hollow optical fiber 151 (an example of a light guide) that guides probe light to the ATR prism 16 is driven by a drive unit. Thus, a detection signal of probe light obtained from the photodetector 17 may be temporally averaged to reduce a variation in measurement of absorbance otherwise occurring due to a probe light speckle and a variation of the output of the light source, a variation of the position of each element due to a vibration of the blood glucose level measuring apparatus, and the like.

FIG. 31 is a diagram illustrating an example of an overall configuration of the blood glucose level measuring apparatus 100a. As depicted in FIG. 31, the blood glucose level measuring apparatus 100a includes a measuring unit la and a processing unit 2a. The measuring unit la includes a piezoelectric drive unit 183 (an example of a drive unit) for driving the first hollow optical fiber 151, and the processing unit 2a includes a drive control unit 23 for controlling the piezoelectric drive unit 183. An absorbance measuring apparatus 101a includes the measuring unit la, the drive control unit 23, and an absorbance obtaining unit 21, as enclosed by a broken line in FIG. 31.

The piezoelectric drive unit 183 includes a piezoelectric element that expands and contracts in predetermined directions in response to input driving voltages. The piezoelectric drive unit 183 is positioned in contact with an intermediate portion in the length direction of the first hollow optical fiber 151 so as to extend and contract in directions intersecting the direction of propagation of probe light through the first hollow optical fiber 151. The first hollow optical fiber 151 is an example of an “optical fiber,” and a position at which one end of the first hollow optical fiber 151 is connected to the ATR prism 16 is an example of a “predetermined position.”

The drive control unit 23 is an electric circuit that outputs a driving signal for driving the piezoelectric drive unit 183 to the piezoelectric drive unit 183. The drive control unit 23 outputs a modulated driving voltage, modulated at a predetermined cycle shorter than a cycle of detecting probe light intensity by the photodetector 17, to the piezoelectric drive unit 183.

FIG. 32 is an enlarged view for illustrating a contact position between the piezoelectric drive unit 183 and the first hollow optical fiber 151.

As depicted in FIG. 32, the piezoelectric drive unit 183 expands and contracts in directions intersecting the direction of propagation of probe light (the directions of the outlined arrow) to change the position of the intermediate portion in the length directions of the first hollow optical fiber 151 in the direction of the outlined arrow. More specifically, the piezoelectric drive unit 183 repeatedly expands and contracts in accordance with the driving voltages input from the drive control unit 23, thereby causing the intermediate portion of the first hollow optical fiber 151 in the length direction to vibrate (to be driven) in the directions of the outlined arrow, thereby changing the position of the intermediate portion periodically.

Because one end of the first hollow optical fiber 151 is connected to the ATR prism 16, the one end of the first hollow optical fiber 151 does not move even though the intermediate portion of the first hollow optical fiber 151 in the longitudinal direction vibrates. Thus, the piezoelectric drive unit 183 can periodically change the position of the intermediate portion in the longitudinal directions of the first hollow optical fiber 151 while the position and angle of incidence of probe light incident on the ATR prism 16 are maintained.

As long as the position of the intermediate portion can be changed, the extending end of the piezoelectric drive unit 183 may be connected to the intermediate portion by adhesion or the like, or the intermediate portion may vibrate as a result of the piezoelectric drive unit 183 periodically contacting the intermediate portion without connection with the intermediate portion.

The frequency of vibration caused by the piezoelectric drive unit 183 is 130 Hz as an example. However, the frequency of vibration is not limited to this value. Vibration at a frequency sufficiently higher than the frequency of detection of probe light intensity by the photodetector 17 is sufficient, and it is desirable to determine an appropriate frequency depending on the weight of the driving target. For a lightweight member such as the first hollow optical fiber 151, a high frequency of 100 kHz or higher may be used. The frequency of detecting the probe light intensity by the photodetector 17 is in a range between 2 Hz and 3 Hz as an example.

In addition, it is desirable that the vibration amplitude of the piezoelectric drive unit 183 be approximately in a range between 1/10 of the beam diameter of probe light and the same as the beam diameter. By vibrating the first hollow optical fiber 151 at this amplitude, the pattern of probe light on the photodetector 17 can be varied and light intensity is integrated at the photodetector 17 so that a time-averaged value can be obtained.

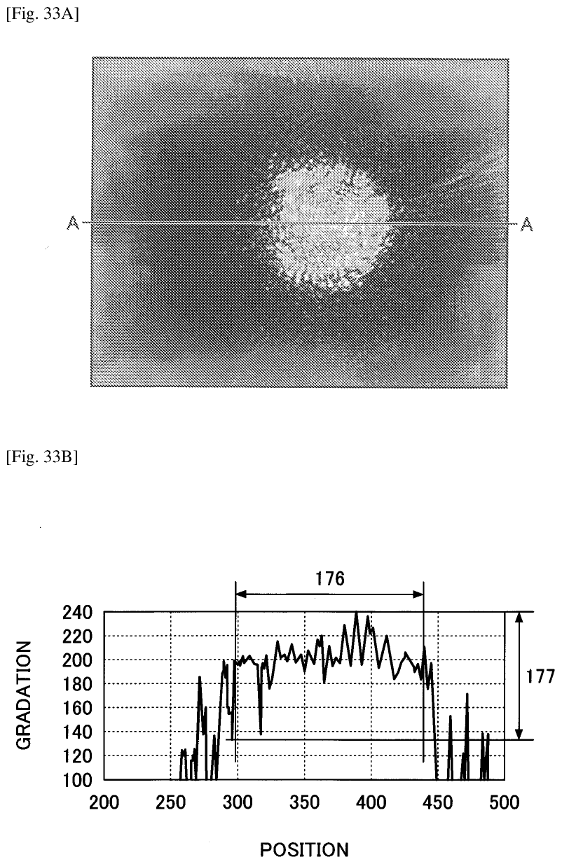

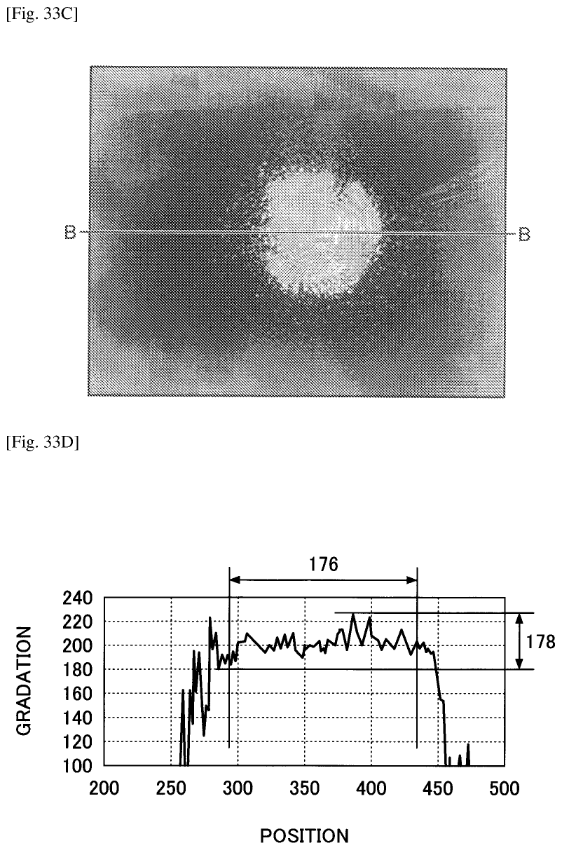

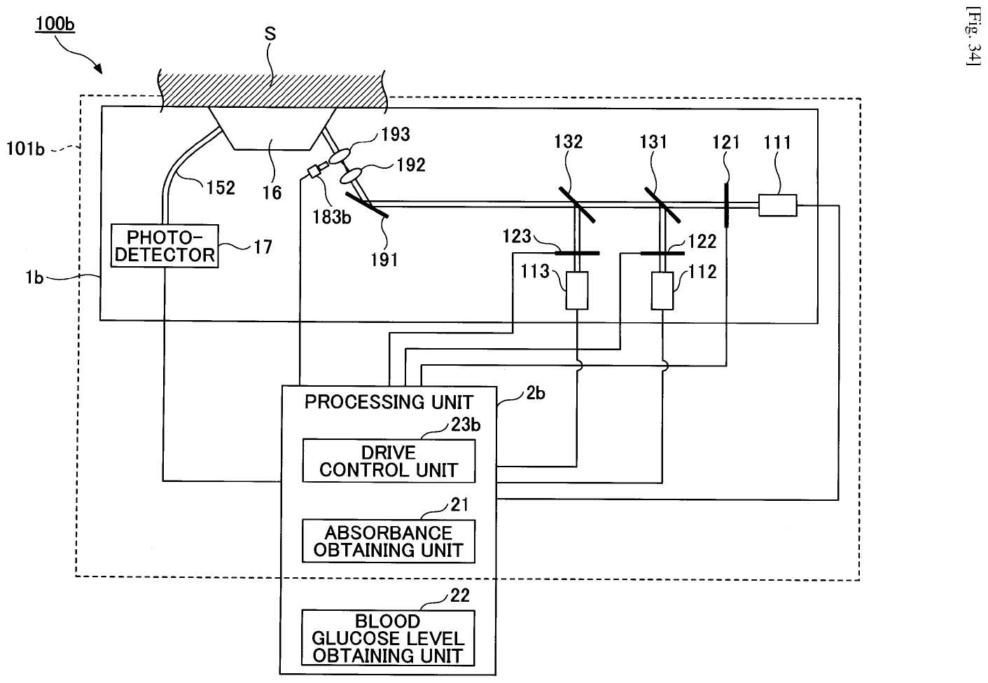

FIGS. 33A-33D are diagrams for illustrating the function of the piezoelectric drive unit 183. FIG. 33A depicts a probe light image in a comparative example, FIG. 33B depicts an A-A cross-sectional light intensity distribution with respect to FIG. 33A, FIG. 33C depicts a probe light image according to the present embodiment, and FIG. 33D depicts a B-B cross-sectional light intensity distribution with respect to FIG. 33C.

The probe light images depicted in FIGS. 33A and 33C are images of probe light emitted from the second hollow optical fiber 152 and captured by an infrared camera, and are used to illustrate the light intensity distributions of probe light detected by the photodetector 17.

With regard to the probe light image depicted in FIG. 33A, the piezoelectric drive unit 183 is not driven and the first hollow optical fiber 151 is not vibrated. In this state, the probe light image remarkably has a spot pattern due to speckles. The A-A cross-sectional light intensity distribution depicted in FIG. 33B includes variations in light intensity corresponding to the speckles, and the variation range of light intensity distribution 177 in the detection range 176 corresponding to the detection range of the photodetector 17 is relatively large as 140 through 240 gradations.

On the other hand, with regard to the probe light image according to the present embodiment depicted in FIG. 33C, the piezoelectric drive unit 183 is driven and the first hollow optical fiber 151 vibrates. In this state, the probe light image varies finely due to the vibration of the first hollow optical fiber 151 in the image capturing cycle of the infrared camera, and the probe light image which is averaged over time of the image capturing cycle of the infrared camera is captured. This time-averaging effect smooths the light intensity distribution, and the variation range of the light intensity distribution 178 in the detection range 176 depicted in FIG. 33D is small as 180 through 230 gradations. Because the first hollow optical fiber 151 is vibrated while the position and angle of incidence of probe light to the ATR prism 16 are maintained, a time-averaging effect can be obtained without changing the position of probe light on the photodetector 17.

By thus reducing the variation of the light intensity distribution, the variation of the detection value of the photodetector 17 is also reduced.

Meanwhile, measurement variations may increase due to variations in the output of the light source that emits probe light, resulting in variations in detection values of the photodetector 17. Measurement variations may also increase as the position of probe light on the photodetector 17 changes due to the positional variations of the elements of the blood glucose level measuring apparatus due to vibrations or the like of the blood glucose level measuring apparatus. Also for such a situation, the time-averaging effect of probe light obtained from vibrations of the first hollow optical fiber 151 described above can reduce the variations in detection values.

<Advantageous Effects of Eighth Embodiment>

As described above, in the present embodiment, the first hollow optical fiber 151, which guides probe light to the ATR prism 16, is driven by the piezoelectric drive unit 183. Accordingly, because a detection signal of the photodetector 17 is thus averaged over time, it is possible to reduce the variations in measurement of absorbance resulting from speckles of probe light, the variations in the output of the light source, and the variations in the position of each element due to vibration of the blood glucose level measuring apparatus. Absorbance can then be accurately measured and blood glucose levels can be accurately measured.

When probe light with low coherence is used, the variations of detection values by speckles are reduced. However, even in this case, the variations of measurement due to output variations of the light source, the positional variations of each element due to vibration of the blood glucose level measuring apparatus or the like are reduced according to the eighth embodiment.

With regard to the present embodiment, an example of vibrating the intermediate portion of the first hollow optical fiber 151 in the length direction is described, but the position of at least a portion of the first hollow optical fiber 151, even other than the intermediate portion, may be changed instead. However, because changing the position of the intermediate portion as described above enables maintaining the position and angle of incidence of probe light on the ATR prism 16, it is desirable to reduce possible measurement errors caused by variations in the probe light intensity due to possible variations in the position and angle of incidence of probe light on the ATR prism 16.

The directions in which the piezoelectric drive unit 183 changes the position of the intermediate portion of the first hollow optical fiber 151 are not limited to the directions perpendicular to the direction of propagation of probe light through the first hollow optical fiber 151. As long as the position of at least a portion of the first hollow optical fiber 151 can be changed as mentioned above, the directions can be any directions. Also, the directions are not limited to fixed directions, but the position of at least a portion of the first hollow optical fiber 151 may be changed in various directions, or the directions in which the position of at least a portion of the first hollow optical fiber 151 is changed may be varied two-dimensionally over time.

With regard to the present embodiment, an example of using the piezoelectric drive unit is described as a drive unit, but the drive unit is not limited to such a piezoelectric drive unit. An ultrasonic vibrator, a voice coil motor, or the like can be used as the drive unit provided that at least one of the position and the angle of the light guide can be changed.

The advantageous effects other than the foregoing are the same as the advantageous effects described above for the seventh embodiment.

<Variations of Eighth Embodiment>

Hereinafter, variants will be described, because there are variants to elements of the present embodiment.

(First Variant)

In a first variant, a light guide that guides probe light to the ATR prism 16 includes a mirror (an example of a deflecting unit) and a lens (an example of a condensing unit). The lens included in the light guide is driven to average a detection signal of the photodetector 17. This reduces the variation in measurement of absorbance due to variation of probe light speckles, variation of the output of the light source, and the positional variation of each element of the blood glucose level measuring apparatus due to vibration.

FIG. 34 is a diagram illustrating an example of the overall configuration of the blood glucose level measuring apparatus 100b according to the present variant. As depicted in FIG. 34, the blood glucose level measuring apparatus 100b includes a measuring unit 1b and a processing unit 2b. The measuring unit 1b includes a deflecting mirror 191 that deflects first through third probe lights toward the ATR prism 16, first and second condenser lenses 192 and 193 that condense the deflected light exiting from the deflecting mirror 191, and a piezoelectric drive unit 183b that drives the second condenser lens 193. The configuration including the deflecting mirror 191, the first condenser lens 192, and the second condenser lens 193 is an example of a light guide. Further, it is desirable that the deflecting mirror 191 be made of a gold or silver material having a high reflectivity with respect to infrared light. In addition, desirably, the first and second condenser lenses 192 and 193 have high condensing efficiency with respect to the mid-infrared region.

The processing unit 2b includes a drive control unit 23b for controlling the piezoelectric drive unit 183b. An absorbance measuring apparatus 101b includes the measuring Unit 1b, the drive control unit 23b, and the absorbance obtaining unit 21, as enclosed by a broken line in FIG. 34.

The piezoelectric drive unit 183b includes a piezoelectric element that expands and contracts in predetermined directions in response to input driving voltages. The piezoelectric drive unit 183b is disposed in contact with a lateral side of the second condenser lens 193 so as to expand and contract in directions intersecting the optical axis of the second condenser lens 193.

The drive control unit 23b is an electrical circuit that outputs a driving voltage for driving the piezoelectric drive unit 183b to the piezoelectric drive unit 183b. The drive control unit 23b outputs a driving voltage modulated at a predetermined cycle shorter than the cycle of detecting probe light intensity by the photodetector 17 to the piezoelectric drive unit 183b.

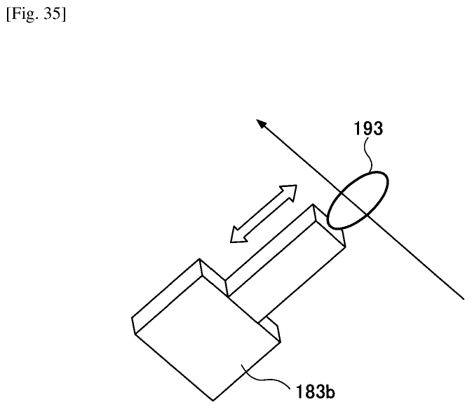

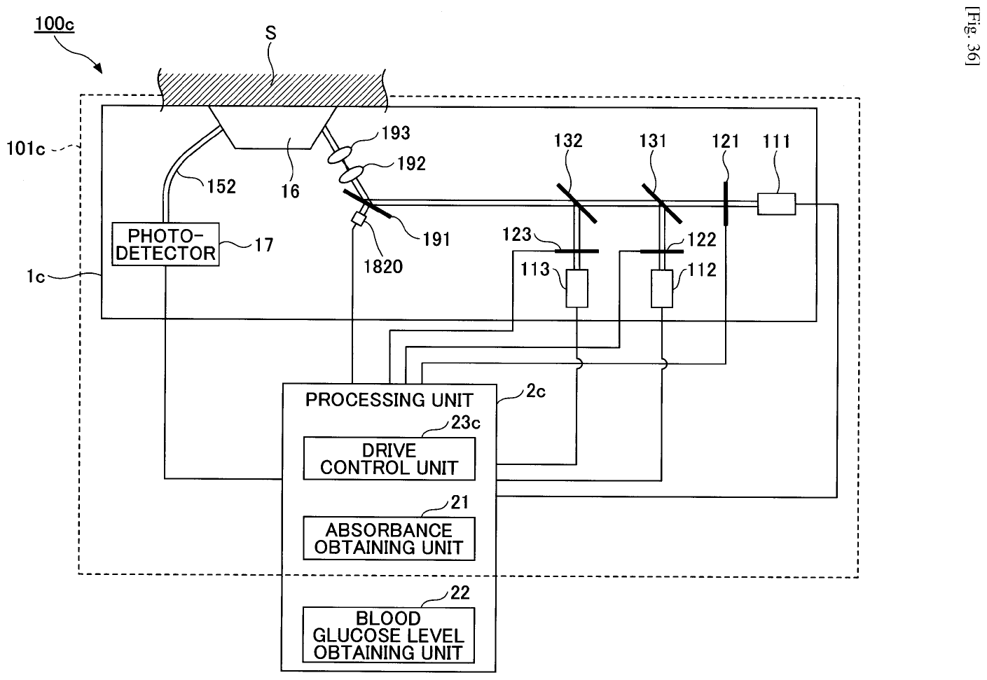

FIG. 35 is an enlarged view for illustrating a driving example of the second condenser lens 193. As depicted in FIG. 35, the piezoelectric drive unit 183b expands and contracts in directions (in the directions of the outlined arrow) intersecting the optical axis of the second condenser lens 193 to change the position of the second condenser lens 193 in the directions of the outlined arrow. More specifically, the piezoelectric drive unit 183b repeatedly expands and contracts in accordance with the driving voltages input from the drive control unit 23b so that the lateral side of the second condenser lens 193 vibrates (is driven) in the directions of the outlined arrow and periodically changes the position of the second condenser lens 193. This causes the position of probe light incident on the photodetector 17 to change finely in a periodic manner as the position of probe light incident on the ATR prism 16 changes periodically.

In this regard, as long as the position of the second condenser lens 193 can be changed, the extending end of the piezoelectric drive unit 183b and the lateral side of the second condenser lens 193 may be connected together by adhesion or the like, or the second condenser lens 193 may be caused to be vibratable by being periodically contacted by the piezoelectric drive unit 183b without being connected with the piezoelectric drive unit 183b.

The frequency of vibration by the piezoelectric drive unit 183b is 130 Hz as an example. However, the frequency of vibration is not limited to this value, and the piezoelectric drive unit 183b may be vibrated at a frequency sufficiently higher than the frequency of detecting probe light intensity by the photodetector 17, and it is desirable to determine an appropriate frequency depending on the weight of the driving target.

Because the second condenser lens 193 is heavier than the first hollow optical fiber 151 of the eighth embodiment (see FIG. 31), it is desirable to use a frequency lower than the frequency at which the first hollow optical fiber 151 is vibrated.

In the eighth embodiment described above, because probe light is vibrated while the position and angle of incidence of probe light on the ATR prism 16 is maintained, the position of probe light on the photodetector 17 does not change even when the first hollow optical fiber 151 is vibrated. However, in the present variant, the position of probe light on the photodetector 17 changes as the position of the probe light incident on the ATR prism 16 changes due to vibration of the second condenser lens 193.

In this regard, the amplitude of the vibration caused by the piezoelectric drive unit 183b is set in a range between 1/10 of the beam diameter of probe light and the same as the beam diameter, so that portions of probe light at the photodetector 17 overlap with each other while the second condenser lens 193 vibrates and the probe light is changed in position. This allows a time-averaging effect to be obtained in the area of overlapping probe light at the photodetector 17.

Because the advantageous effects of the blood glucose level measuring apparatus 100b according to the present variant are the same as the advantageous effects described above for the eighth embodiment, the duplicate description will be omitted.

In the present variant, the piezoelectric drive unit 183b contacts the lateral side of the second condenser lens 193 to vibrate the second condenser lens 193. However, the piezoelectric drive unit 183b may contact a holding unit (not depicted) holding the second condenser lens 193 to vibrate the second condenser lens 193 via the holding unit.

Further, although the example of using the piezoelectric drive unit as a drive unit has been described for the present variant, the drive unit is not limited to this example. An ultrasonic vibrator, a voice coil motor, or the like can be used as the drive unit provided that at least one of the position and angle of the light guide can be changed by the drive unit.

(Second Variant)

In the first variant, the second condenser lens 193 included in the light guide is driven. In a second variant, the deflecting mirror 191 included in the light guide is driven, and a detection signal of the photodetector 17 with respect to probe light is averaged over time. This reduces the variation in measurement of absorbance due to probe light speckles, the variations in output of the light source, and the positional variations of each element of the blood glucose level measuring apparatus due to vibration.

FIG. 36 is a diagram illustrating an example of the overall configuration of the blood glucose level measuring apparatus 100c according to the present variant. As depicted in FIG. 36, the blood glucose level measuring apparatus 100c includes a measuring unit 1c and a processing unit 2c. The measuring unit 1c includes a deflecting mirror 191 that deflects first through third probe lights toward the ATR prism 16, a first condenser lens 192 and a second condenser lens 193 that condense light deflected by the deflecting mirror 191, and a piezoelectric drive unit 1820 that drives the deflecting mirror 191.

The processing unit 2c includes a drive control unit 23c for controlling the piezoelectric drive unit 1820. An absorbance measuring apparatus 101c includes the measuring unit lc, the drive control unit 23b, and an absorbance obtaining unit 21, as enclosed by a broken line in FIG. 36.

The piezoelectric drive unit 1820 includes a piezoelectric element that expands and contracts in predetermined directions in response to input driving voltages. The piezoelectric drive unit 1820 is disposed in contact with a back portion of the deflecting mirror 191 to extend and contract in directions perpendicular to the mirror surface of the deflecting mirror 191.

The drive control unit 23c is an electrical circuit that outputs a driving voltage for driving the piezoelectric drive unit 1820 to the piezoelectric drive unit 1820. The drive control unit 23c outputs a driving voltage modulated at a predetermined cycle shorter than the cycle of detection of probe light intensity by the photodetector 17 to the piezoelectric drive unit 1820.

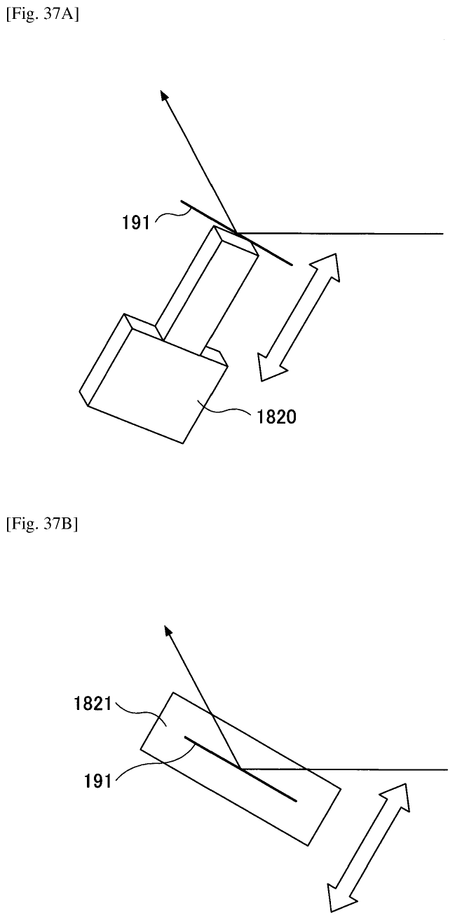

FIGS. 37A-37C are diagrams illustrating a driving example of the deflecting mirror 191. FIG. 37A depicts a case in which the piezoelectric drive unit 1820 is vibrated by a driving source, FIG. 37B depicts a case in which a motor 1821 is vibrated by a drive source, and FIG. 37C depicts a case in which the piezoelectric drive unit 1820 is oscillated by a micro mechanical electro system (MEMS) mirror 1822.

As depicted in FIG. 37A, the piezoelectric drive unit 1820 extends and contracts in directions perpendicular to the mirror surface of the deflecting mirror 191 (in the directions of the outlined arrow) to change the position of the deflecting mirror 191 in the directions of the outlined arrow. The piezoelectric drive unit 1820 repeatedly expands and contracts in accordance with the driving voltages input from the drive control unit 23c to cause the deflecting mirror 191 to vibrate (be driven) in the directions of the outlined arrow and to periodically change the position of the deflecting mirror 191. This causes the position of probe light incident on the ATR prism 16 to be varied and the position of probe light on the photodetector 17 to be varied finely periodically.

In this regard, as long as the position of the deflecting mirror 191 can be changed, the extending end of the piezoelectric drive unit 1820 and the back portion of the deflecting mirror 191 may be connected together by adhesion or the like, or the deflecting mirror may be caused to be vibratable through periodical contacting of the piezoelectric drive unit 1820 without connection between these members.

As depicted in FIG. 37B, the motor 1821 vibrates in directions perpendicular to the mirror surface of the deflecting mirror 191 (in the directions of the outlined arrow) to change the position of the deflecting mirror 191 in the directions of the outlined arrow. The motor 1821 is a motor, such as a ring-shaped (hollow) voice coil motor. The motor 1821 holds the deflecting mirror 191 inside the ring and vibrates in the directions of the outlined arrow according to the driving voltages input from the drive control unit 23c to cause the deflecting mirror 191 to vibrate in the directions of the outlined arrow and to periodically change the position of the deflecting mirror 191. This causes the position of probe light incident on the ATR prism 16 to be varied and the position of probe light on the photodetector 17 to be varied finely periodically.

As depicted in FIG. 37C, the MEMS mirror 1822 is a mirror in which a drive unit, such as a piezoelectric drive unit, is integrally formed by a semiconductor process. The piezoelectric drive unit deforms according to a driving voltage input from the drive control unit 23c, causing the deflecting mirror 191 to rotate about an axis parallel to the mirror surface (for example, an axis perpendicular to the plane of the paper in FIG. 37C), thereby changing the angle of the deflecting mirror 191. This causes the deflection angle of probe light by the deflecting mirror 191 to change, the position of probe light incident on the ATR prism 16 to change, and the position of probe light on the photodetector 17 to change periodically and finely.

The driving frequency, the amplitude of the driving, and the advantageous effects are the same as the driving frequency, the amplitude of the driving, and the advantageous effects of the first variant, and thus the duplicate description will be omitted.

With regard to the present variant, the examples of the piezoelectric drive unit, voice coil motor, MEMS mirror, and so forth have been described as drive units, but the drive unit is not limited to these examples. An ultrasonic vibrator, an acousto-optic device, a polygon mirror, or the like may be used as the drive unit, provided that at least one of the position and angle of the light guide can be varied by the drive unit.

(Third variant)

With regard to the first and second variants, examples in which the light guide is driven to reduce measurement variations caused by speckles of probe light have been described. Because speckles are generated by interference of scattered light of probe light or the like, generation of speckles can be reduced by reducing the coherence of probe light. Therefore, in a third variant, by superimposing a high frequency modulation component with a current driving the light source, the coherence of the light source included in the blood glucose level measuring apparatus is reduced, and measurement variations of absorbance due to speckles of probe light is reduced.

FIGS. 14A and 14B, described above, are again used as diagrams illustrating examples of light source driving currents according to the present variant. FIG. 14A depicts a light source driving current according to a comparative example, and FIG. 14B represents a high frequency modulated light source driving current according to the present variant.

The light source control unit 212 (see FIG. 6) periodically outputs a pulsed driving current as depicted in FIG. 14A to each of the first light source 111, the second light source 112, and the third light source 113 to emit pulsed probe light.

In the present variant, a high-frequency modulated component is superimposed with the pulsed driving current of FIG. 14A to output to the first light source 111, the second light source 112, and the third light source 113. The waveform of the high-frequency modulated component may be of a sinusoidal wave or a rectangular wave. The modulation frequency can be any from among 1 MHz (megahertz) through several GHz (gigahertz).

By superimposing a high frequency modulated component, the first light source 111, the second light source 112, and the third light source 113 can emit pseudo multimode laser light as probe light, respectively, to reduce the coherence of probe light. This reduces speckles of probe light due to reduced coherence and decreases the measurement variations due to speckles.

Thus, the eighth embodiment and the first through third variants of the eighth embodiment have been described. In this regard, it is also possible to combine some elements of these embodiment and variants to implement an absorbance measuring apparatus or a blood glucose level measuring apparatus.

In addition, with regard to the above-described examples, the examples of applying the present embodiment and variants to a blood glucose level measuring apparatus have been described, but application of the present embodiment and variants is not limited to this application. The embodiment and variants are also applicable to light guide devices including light guides for guiding probe light, drive units for driving light guides, and control units for controlling drive units. Such light guide devices can obtain the same advantageous effects as the advantageous effects of the above-described absorbance measuring apparatuses.

### Ninth Embodiment

Next, a blood glucose level measuring apparatus according to a ninth embodiment will be described.

The ninth embodiment is similar to the first embodiment described above with reference to FIGS. 1-14B. Therefore, mainly, the points different from the first embodiment will be described, and duplicate description may be omitted.

In the present embodiment, by limiting the measurement sensitivity area in the ATR prism 16, the variation in measurement of absorbance due to a variation in the contact area between the ATR prism 16 and a living body S for each measurement is reduced.

The term “measurement sensitivity area” refers to an area of the total reflection face having measurement sensitivity for measurement on the basis of the ATR method. More specifically, the term “measurement sensitivity area” refers to an area at which an attenuation, caused by a living body, of a field penetrating from the total reflection face, can be caused to occur.

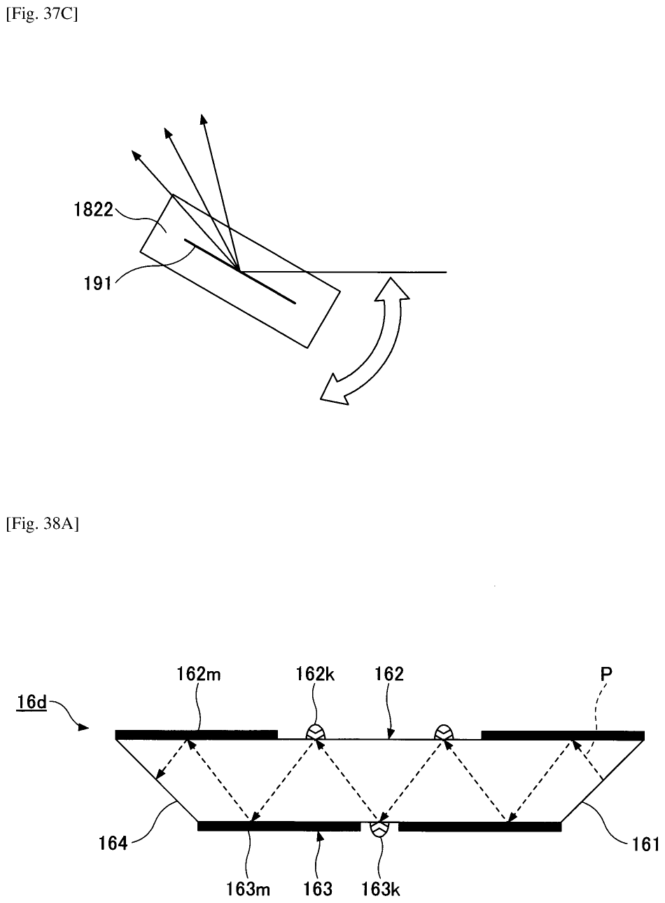

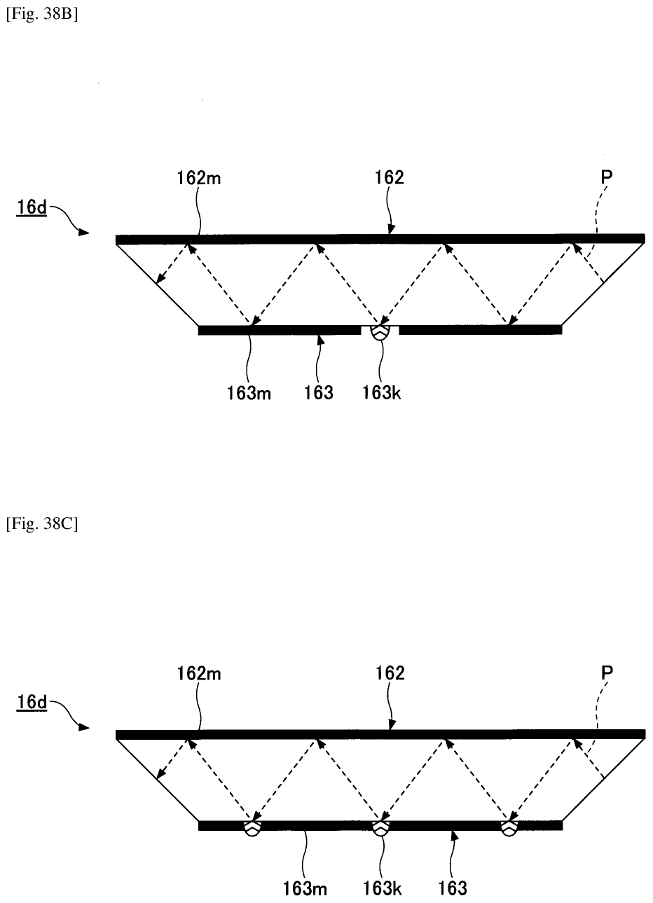

FIGS. 38A-38C are diagrams illustrating a configuration example of an ATR prism 16d in which a measurement sensitivity area is defined in accordance with the present embodiment. FIGS. 38A-38C depict three examples of different measurement sensitivity areas. In FIGS. 38A-38C, probe light P, represented by the broken arrows, is incident from the incidence face 161 of the ATR prism 16d and undergoes total reflection four times by the first total reflection face 162 and three times by the second total reflection face 163, before exiting from the outgoing face 164.

An area of the first total reflection face 162 in each figure is provided with a reflective film 162m made of gold or silver with a high reflectivity to infrared rays. An area of the second total reflection face 163 is similarly provided with a reflective film 163m made of gold or silver with a high reflectivity to infrared light. Such reflective films 162m and 163m can be formed by vapor deposition of gold or silver on the total reflection face. When a mask is used for vapor deposition, gold or silver can be formed through vapor deposition in areas other than the masked areas.

In the areas where the reflective films 162m and 163m are provided in the first and second total reflection faces 162 and 163, total reflection does not occur and penetration of a field does not occur. Therefore, these areas cannot function as measurement sensitivity areas because an attenuation of the field caused by a living body S does not occur. In other words, each of the reflective films 162m and 163m has the function of defining a measurement sensitivity area in the total reflection face. The reflective films 162m and 163m are examples of area defining sections. The areas where the reflective films 162m and 163m are provided in the first total reflection face 162 and the second total reflection face 163 are examples of “an area other than a measurement sensitivity area”, while the areas where the reflective films 162m and 163m are not provided are examples of “an area other than an edge”.

FIG. 38A depicts a case where measurement sensitivity areas are provided at both of the first and second total reflection faces 162 and 163. At each of the first and second total reflection faces 162 and 163, the reflective films 162m and 163m are provided at the areas other than the center area. The center areas without the reflective films 162m and 163m correspond to the measurement sensitivity areas.

The fields 162k indicated as being filled with diagonal hatching represent fields penetrating from the first total reflection face 162. Because of two times of total reflection, the two fields 162k are generated. Similarly, the field 163k represents a field penetrating from the second total reflection face 163. One time of total reflection generates the field 163k at one point.

FIG. 38B depicts a case where there is a measurement sensitivity area at a center area of the second total reflection face 163. Because the first total reflection face 162 is provided with a reflective film 162m throughout the entire face, the first total reflection face 162 does not have a measurement sensitivity area. The second total reflection face 163 is provided with a reflective film 163m except at the center area. The field 163k is generated at the center area which thus acts as the measurement sensitivity area.

FIG. 38C depicts a case where measurement sensitivity areas are at multiple points (in this example, three points) in the second total reflection face 163. Because the first total reflection face 162 is provided with a reflective film 162m throughout the entire surface, the first total reflection face 162 does not have a measurement sensitivity area. The second total reflection face 163 is provided with reflective films 163m except for the above-mentioned three points. At the three points having no reflective film 163m, fields 163k are generated, and thus, these three areas function as the measurement sensitivity areas.

In blood glucose level measurement using the ATR prism, a to-be-measured person may put the ATR prism 16d in the mouth in such a way that the first total reflection face 162 contacts the upper lip of the living body S of the to-be-measured person and the second total reflection face 163 contacts the lower lip of the living body S. In this case, the center of the lip is easy to apply holding force, allowing the lip to be in relatively stable contact with the ATR prism. On the other hand, near any one of both ends of the lip, measurement variations may increase due to variations in the contact area because of the relative difficulty of applying holding force to the lip or individual variations in the size of the mouth.

In this regard, in the example of FIG. 38A, the measurement sensitivity areas near both ends of the ATR prism 16d in contact with both ends of the lip can be covered by the reflective films 162m and 163m, so that the areas in which a contact point tends to vary can be caused not to be used for measurement.

The areas provided with the reflective films 162m in FIG. 38A correspond to both ends of the first total reflection face 162. However, a reflective film 162m may be provided at either one end. The areas where the reflective films 163m are provided in FIG. 38A corresponds to both ends of the second total reflection face 163. However, a reflective film 163m may be provided at either one end.

In addition, because the lower lip is easier to apply holding force to the ATR prism 16d than the upper lip, a measurement variation for absorbance may be reduced by using only the second total reflection face 163, which is contacted by the lower lip.

In the example of FIG. 38B, the measurement sensitivity areas are defined by the reflective films 162m and 163m by covering of the entire surface of the first total reflection face 162 contacting the upper lip and covering of near both ends of the second total reflection face 163 contacting both ends of the lower lip, so that only areas at which the contact areas are unlikely to vary are used for measurement.

In addition, the greater the number of total reflections from the total reflection faces is, the greater the attenuation caused by a living body S is, and the higher the sensitivity of measurement is. In the example of FIG. 38C, the three areas where total reflection occurs at the second total reflection face 163 where the lower lip contacts are used as areas where reflective films 163m are not formed. As a result, a blood glucose level can be measured with the total number of reflections (three times) that is greater than the total number of reflections (one time) of the case of FIG. 38B, and high accuracy measurement with higher measurement sensitivity can be achieved.

The blood glucose level measuring apparatus 100 according to the seventh embodiment is applicable to the overall configuration of the blood glucose level measuring apparatus according to the present embodiment, with the ATR prism 16 being replaced with the ATR prism 16d.

The areas where total reflection occurs at the first total reflection face 162 and the second total reflection face 163 can be identified experimentally or through simulation on the basis of the angle of incidence of probe light on the ATR prism 16. Then, reflective films 162m and 163m can be provided at the areas other than the thus identified areas where total reflection occurs.

### <Advantageous Effects of Ninth Embodiment>

Depending on the contact area of a living body to the ATR prism, the area of generation of a field that penetrates from the total reflection face of the ATR prism varies. When measuring a blood glucose level, it is desirable that the contact area be constant. However, in practice, it is difficult to precisely make the contact area of a living body to the ATR prism be constant for each measurement, so the contact area may vary from measurement to measurement, and variation in absorbance may increase due to variation in the contact area. Especially, when a lip is used as a measurement target portion, the contact area is easily changed near a lip edge depending on an individual difference in the lip size and the degree of application of force to hold the ATR prism, and thus, a measurement variation is likely to occur.

In the present embodiment, the measurement sensitivity areas of the ATR prism 16d are defined by the reflective films 162m and 163m as area defining sections. Accordingly, an area in which the contact area in the ATR prism 16d is easily variable is not used for measurement, but only an area in which the contact area is relatively unlikely to vary can be used for measurement. As a result, a variation in measurement of absorbance due to a variation in the contact area between the ATR prism 16d and a living body S can be reduced, and a variation in measurement of a blood glucose level can be reduced.

### Tenth Embodiment

Next, a blood glucose level measuring apparatus according to a tenth embodiment will be described. The tenth embodiment is similar to the first embodiment described above with reference to FIGS. 1-14B. Therefore, mainly, the points different from the first embodiment will be described, and duplicate description may be omitted.

In the present embodiment, the contact pressure (pressure) of a living body S on the ATR prism 16 is detected by a pressure sensor (an example of a pressure detector). By obtaining absorbance data with respect to probe light on the basis of the light intensity of the probe light and the contact pressure detected by the pressure sensor, a variation in measurement of absorbance due to a variation in the contact pressure for each measurement is reduced and a variation in measurement of a blood glucose level is reduced.

<Example of Layout of Pressure Sensor 30>

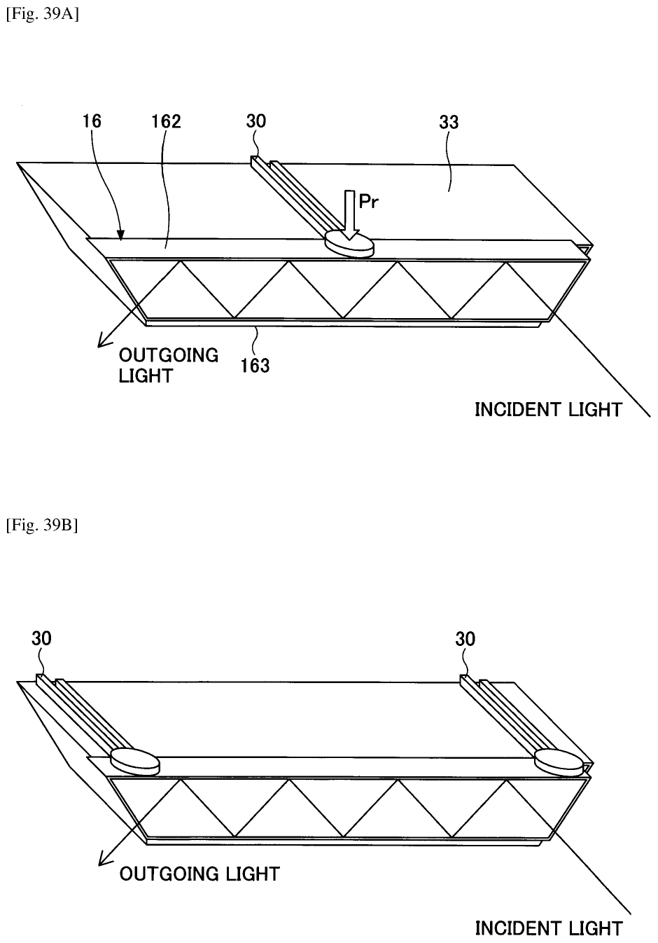

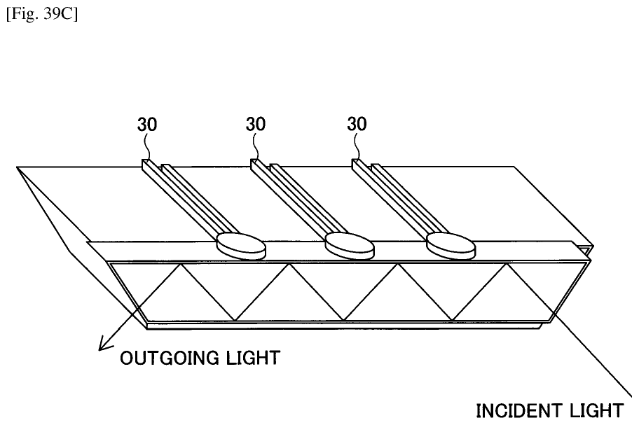

FIGS. 39A-39C are diagrams illustrating examples of arrangements of pressure sensors 30 at the ATR prism 16. FIGS. 39A-39C depict three examples of different layouts and numbers of pressure sensors 30. FIG. 39A depicts a case where one pressure sensor 30 is provided, FIG. 39B depicts a case where pressure sensors 30 are provided at both ends of the ATR prism 16, and FIG. 39C depicts a case where a plurality (in this example, three) of pressure sensors 30 are provided.

As depicted in the figures, a total reflection support 33 contacts a side face of the ATR prism 16 (other than the incident and outgoing faces with respect to probe light) to support the ATR prism 16 and supports the pressure sensor(s) 30 on the first total reflection face 162.

The pressure sensors 30 are fixed by adhesion or the like in contact with at least one of the ATR prism 16 and the total reflection support 33. The pressure sensors 30 are sensors that detect the contact pressure Pr received by the ATR prism 16 from a lip when a to-be-measured person as a living body S has put the ATR prism 16 in the mouth. Any one of various types of pressure sensors may be used as the pressure sensors 30, such as a capacitive sensor, a strain gauge sensor, a pressure-sensitive resistance sensor whose resistance value varies with pressure, and a pressure sensor utilizing MEMS technology.

Although FIG. 39A-39C depict examples in each of which the pressure sensor(s) 30 is(are) disposed only on the first total reflection face 162 of the ATR prism 16, the pressure sensor(s) 30 may be disposed on at least one of the first total reflection face 162 and the second total reflection face 163 of the ATR prism 16.

As depicted in FIG. 39B, when the pressure sensors 30 are provided near both ends of the ATR prism 16, the pressures, near both ends of the lip where the contact pressure is easy to vary because it is relatively hard to apply a force to hold or the size of the mouth varies from person to person, can be detected. Further, as depicted in FIG. 39C, when the three pressure sensors 30 are provided, a distribution of contact pressures can be detected.

When the pressure sensor(s) 30 is(are) positioned at the total reflection face, the area where the pressure sensor(s) 30 is(are) positioned is not a measurement sensitivity area because penetration of a field from the total reflection face does not occur and an attenuation of a penetrating field caused by a living body S does not occur.

Therefore, the pressure sensor(s) 30 can be provided as an area defining section(s) described above with regard to the ninth embodiment, and the pressure sensor 30 is disposed in an area in which a contact area easily vary, such as the vicinity of both ends of the ATR prism 16 or the like. Therefore, it is possible to reduce a variation in measurement of absorbance due to a variation in a contact area.

In this regard, if the pressure sensors 30 were placed at all the areas where total reflection occurs in the ATR prism 16, measurement on the basis of the ATR method would not be possible, so it is desirable not to place pressure sensors 30 in at least some of the areas where total reflection occurs to secure a measurement sensitivity area.

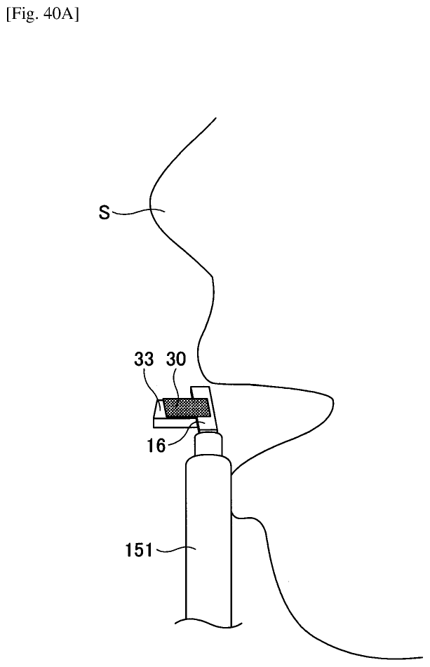

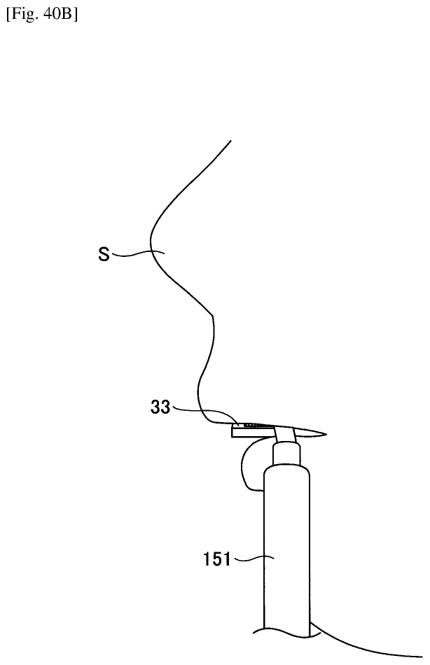

FIGS. 40A and 40B are diagrams illustrating an example of the ATR prism 16 and the pressure sensor 30 positioned at lips. FIG. 40A depicts a state of before contact with the lips, and FIG. 40B depicts a state where a person puts the ATR prism 16 in the mouth.

As can be seen in FIGS. 40A and 40B, the size of the ATR prism 16 is small relative to the lips of a person as a living body S. As a result, when the person puts the ATR prism 16 in the mouth, the lips are accessible to both the ATR prism 16 and the total reflection support 33. Accordingly, although FIG. 39A-39C illustrate the examples in which the pressure sensor(s) 30 is(are) disposed at both the total reflection face of the ATR prism 16 and the total reflection support 33, the pressure sensor(s) 30 may be disposed and fixed only to the total reflection support 33.

<Functional Configuration of Processing Unit 2d>

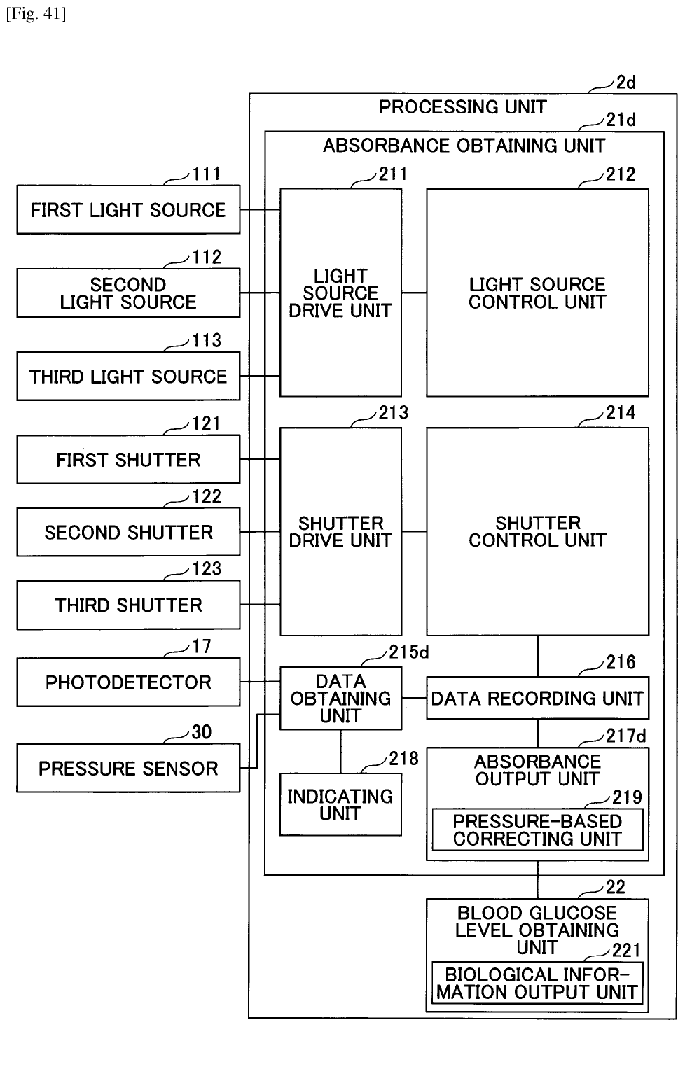

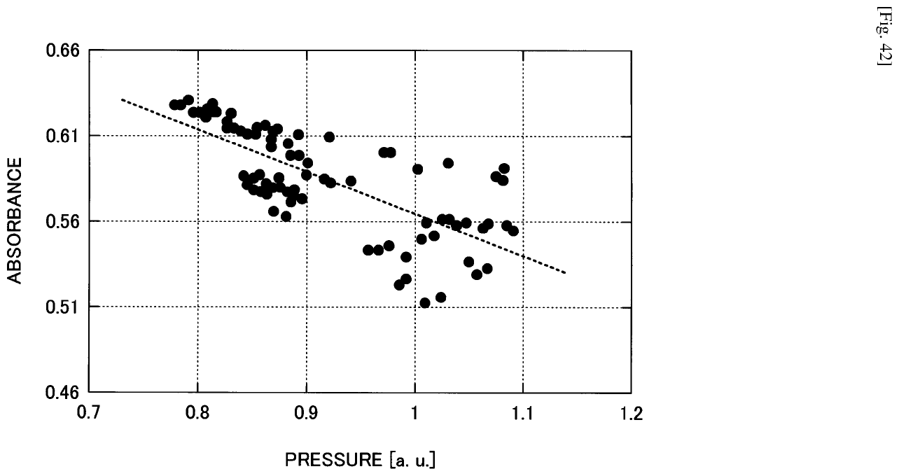

Next, a functional configuration of a processing unit 2d provided in the blood glucose level measuring apparatus according to the present embodiment will be described. FIG. 41 is a block diagram illustrating an example of a functional configuration of the processing unit 2d. As depicted in FIG. 41, the processing unit 2d includes an absorbance obtaining unit 21d, and the absorbance obtaining unit 21d includes a data obtaining unit 215d, an indicating unit 218, and an absorbance output unit 217d. The absorbance output unit 217d includes a pressure-based correcting unit 219.

The function of the data obtaining unit 215d is implanted by the detecting I/F 519 (see FIG. 5) or the like, and the function of the indicating unit 218 is implemented by the display 506 or the like. The functions of the absorbance output unit 217d and the pressure-based correcting unit 219 are implemented by executing of predetermined programs by the CPU 501 or the like.

The data obtaining unit 215d samples a detection signal continuously output by the photodetector 17 at a predetermined sampling cycle and outputs a detection value of the obtained light intensity to the data recording unit 216. At the same time, a detection signal continuously output by the pressure sensor 30 is sampled at a predetermined sampling cycle, and contact pressure data thus obtained is output to the indicating unit 218. However, the data obtaining unit 215d may output contact pressure data to the indicating unit 218 through the data recording unit 216.

The indicating unit 218 displays contact pressure data on the display 506 so that a person putting the ATR prism 16 in the mouth can see the contact pressure data. The person putting the ATR prism 16 in the mouth can adjust the contact pressure between the ATR prism 16 and his/her lips while visually recognizing the contact pressure data displayed on the display 506.

However, indicating of the contact pressure is not limited to such a display of contact pressure by the indicating unit 218. Indicating of a contact pressure may be implemented in such a manner that, in response to contact pressure data exceeding a predetermined contact pressure threshold, a beep may be generated and a message may be displayed on the display 506 indicating that contact pressure exceeds the threshold.

The absorbance output unit 217d performs a predetermined calculation process on the basis of detection values of probe light intensity read from the data recording unit 216 and obtains absorbance data. The pressure-based correcting unit 219 of the absorbance output unit 217d corrects the absorbance data by referring to a table indicating correspondence relationships between a contact pressure and absorbance obtained in advance. The absorbance output unit 217d outputs the corrected absorbance data to the blood glucose level obtaining unit 22. The absorbance output unit 217d is an example of “an absorbance output unit configured to output absorbance of probe light obtained on the basis of light intensity of probe light and a pressure.”

Either one of the indicating by the indicating unit 218 and the correcting of absorbance data by the pressure-based correcting unit 219 may be performed, or both of the indicating by the indicating unit 218 and the correcting of absorbance data by the pressure-based correcting unit 219 may be performed in combination.

FIG. 42 is a diagram illustrating an example of a correspondence between a contact pressure to the ATR prism 16 by a lip and absorbance. The horizontal axis and the vertical axis of FIG. 42 depict a contact pressure and absorbance, respectively. The correspondence relationships depicted in FIG. 42 were experimentally obtained. The pressure sensor used in this experiment was of a pressure-sensitive resistance type.

A table corresponding to the data depicted in FIG. 42 is stored in a storage device such as the HD 504 (see FIG. 5), and the pressure-based correcting unit 219 corrects absorbance data by referring to the table on the basis of obtained contact pressure data.

As depicted in FIG. 42, because a contact pressure and absorbance have a linear relationship, a linear equation corresponding to the linear relationship may be stored in the HD 504, and the pressure-based correcting unit 219 may correct absorbance data using the linear equation on the basis of obtained contact pressure data.

<Example of Placement of Pressure Sensor on Total Reflection Support 33>

As noted above, the ATR prism 16 is small relative to to-be-measured person's lips, so that when a person puts the ATR prism 16 in the mouth, the lips are accessible to both the ATR prism 16 and the total reflection support 33. Therefore, the pressure sensor need not be positioned at both the ATR prism 16 and the total reflection support 33, but the pressure sensor 30 may be positioned only on the total reflection support 33 to detect the contact pressure between the lip and the ATR prism 16.

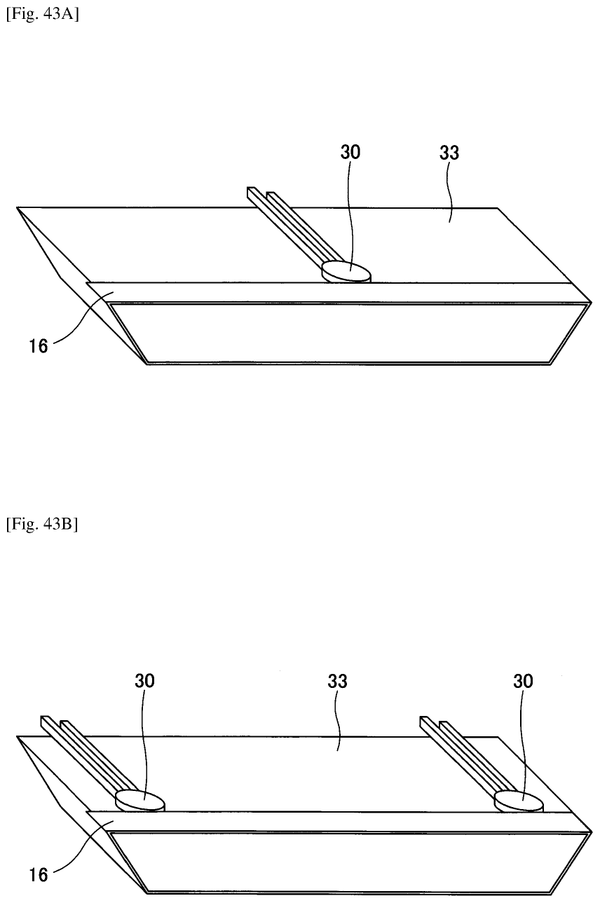

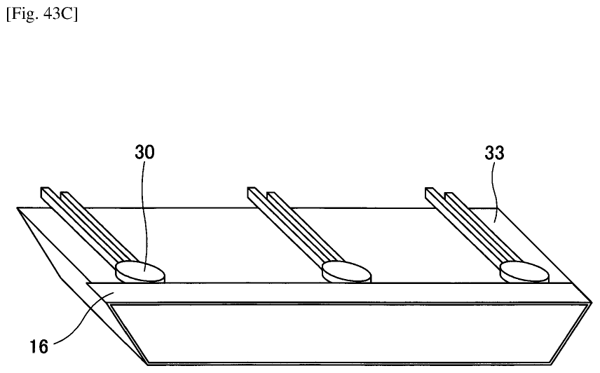

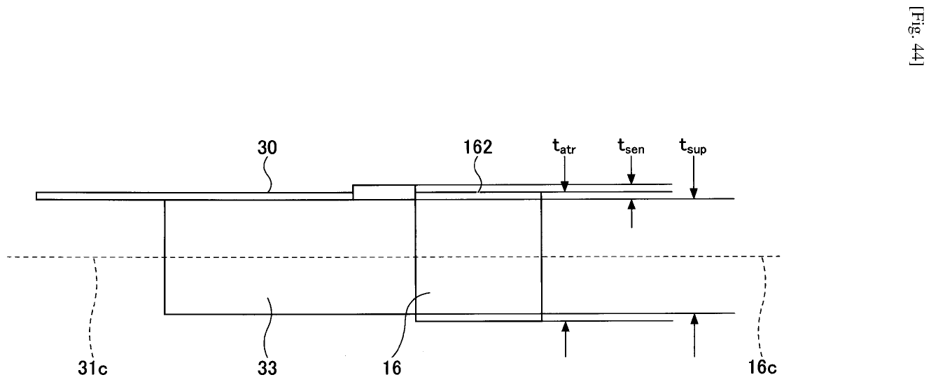

FIGS. 43A-43C are diagrams illustrating an example in which the pressure sensor 30 is disposed only on the total reflection support 33. FIGS. 43A-43C illustrate three examples of different placement positions and numbers of pressure sensors 30. FIG. 43A depicts a case where one pressure sensor 30 is provided, FIG. 43B depicts a case where two pressure sensors 30 are provided at both ends of the ATR prism 16, and FIG. 43C depicts a case where a plurality (in the example, three) of pressure sensors 30 are provided.

As illustrated in FIG. 43B, when the pressure sensors 30 are provided at both ends of the ATR prism 16, the contact pressures near the ends of the lips where the contact pressures are variable because it is relatively hard to apply holding force and there may be an individual variation in the size of the mouth can be detected. In case of using the three pressure sensors 30, as depicted in FIG. 43C, the distribution of contact pressure can be detected by using three pressure sensors 30.

FIG. 44 is a diagram illustrating an example of positional relationships in the thickness direction between the pressure sensor 30, the total reflection support 33, and the ATR prism 16. FIG. 44 depicts a side view (along the longitudinal direction of the ATR prism 16) of a state where the pressure sensor 30 is placed on the total reflection support 33 and a side face of the ATR prism 16 is in contact with and joined to the total reflection support 33.

In FIG. 44, tₐₜᵣ represents the thickness of the ATR prism 16, tₛₑₙ represents the thickness of the pressure sensor 30, and tₛᵤₚ represents the thickness of the total reflection support 33.

In this case, it is desirable to determine the thickness of each element to satisfy the following expression (1).

[Math.1]

tₛᵤₚ<t ₐₜᵣ (1)

As a result of the relationships of the expression (1) being satisfied, a lip can be firmly in contact with the total reflection face of the ATR prism 16 while the total reflection support 33 is prevented from inhibiting contact between the lip and the total reflection face of the ATR prism 16.

In addition, it is desirable to determine the thickness of each element so as to satisfy the following expression (2) when the centerline 16c of the ATR prism 16 with respect to the thickness direction is the same as the centerline 31c of the total reflection support 33 with respect to the thickness direction.

[Math.2]

(tₐₜᵣ−tₛᵤₚ)/2≤tₛₑₙ (2)

As a result of the relationships of the expression (2) being satisfied, the sensor surface of the pressure sensor 30 can be caused to protrude slightly in the thickness direction with respect to the first total reflection face 162 of the ATR prism 16, allowing the contact pressure of the lip to the ATR prism 16 to be suitably detected by the pressure sensor 30.

However, if the protruding amount is too large, it may be impossible for the lip to be in proper contact with the ATR prism 16. Therefore, it is desirable to determine the thickness of each element so as to satisfy the following expression (3).

[Math.3]

0≤tₛₑₙ−(tₐₜᵣ−tₛᵤₚ)/2<(mm) (3)

As a result of the relationships of the expression (3) being satisfied, the sensor surface of the pressure sensor 30 can be prevented from protruding too much relative to the first total reflection face 162 of the ATR prism 16, making the contact pressure of the lip to the ATR prism 16 more desirably detectable by the pressure sensor 30.

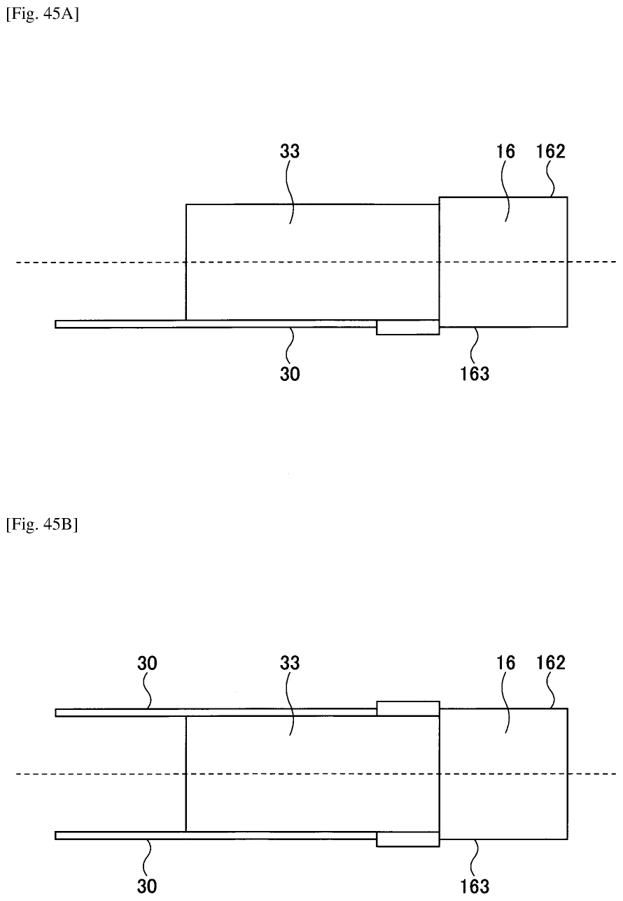

FIGS. 45A and 45B illustrate other examples of positional relationships in the thickness direction between the pressure sensor 30, the total reflection support 33, and the ATR prism 16. Similar to FIG. 44, FIGS. 45A and 45B depict a side view (along the longitudinal direction of the ATR prism 16) of a state where the pressure sensor 30 is placed on the total reflection support 33 and a side face of the ATR prism 16 is in contact with and fixed to the total reflection support 33.

FIG. 45A depicts a case where the pressure sensor 30 is disposed on the second total reflection face 163 side, and FIG. 45B depicts a case where the pressure sensors 30 are disposed on both of the first total reflection face 162 side and the second total reflection face 163 side.

Also in the arrangements of FIGS. 45A and 45B, it is desirable to determine the thickness of each element so as to satisfy expressions (1)-(3) in the same manner as described above.

### <Advantageous Effects of Tenth Embodiment>

As described above, in the present embodiment, a contact pressure of a living body S on the ATR prism 16 is detected by a pressure sensor 30, and absorbance data with respect to probe light is obtained on the basis of a detection value of probe light intensity obtained by the photodetector 17 and the contact pressure.

More specifically, in the present embodiment, contact pressure data is displayed on the display 506 and indicated to a to-be-measured person so that the person putting the ATR prism 16 in the mouth can visually recognize the data. This allows the person putting the ATR prism 16 in the mouth to adjust the contact pressure between the ATR prism 16 and his/her lip while viewing the contact pressure data displayed on the display 506. As a result, it is possible to reduce a variation in a contact pressure at each measurement, to reduce a variation in measurement of absorbance caused by a contact pressure variation, and to reduce a variation in measurement of a blood glucose level.

In the present embodiment, absorbance data is corrected by referring to data indicating correspondence relationships between a contact pressure and absorbance, and the corrected absorbance data is output to the blood glucose level obtaining unit 22. Thus, it is possible to reduce a variation in a contact pressure at each measurement, reduce a variation in measurement of absorbance occurring due to the variation, and reduce a variation in measurement of a blood glucose level.

In addition, both a process of indicating a contact pressure and a process of correcting absorbance data on the basis of contact pressure data can reduce a variations in measurement of absorbance occurring due to a variation in a contact pressure at each measurement, thereby reducing a variation in measurement of a blood glucose level. By performing both of these processes, the correction accuracy can be ensured as a result of the time required for a to-be-measured person to adjust a contact pressure performed being able to be reduced and the amount to be corrected being able to be reduced.

The pressure sensor 30 may be provided on at least one of the ATR prism 16 and the total reflection support 33.

### Eleventh Embodiment

Next, a blood glucose level measuring apparatus according to an eleventh embodiment will be described.

The eleventh embodiment is similar to the first embodiment described above with reference to FIGS. 1-14B. Therefore, mainly, the points different from the first embodiment will be described, and duplicate description may be omitted.

In the present embodiment, by obtaining blood glucose level data on the basis of light intensity of probe light and the temperature of at least one of a living body S and the ATR prism 16, the influence of heat of the ATR prism 16 on the living body S is reduced, and the influence of heat of the living body S on the ATR prism 16 is reduced, thereby accurately measuring a blood glucose level.

<Function Configuration of Processing Unit 2e>

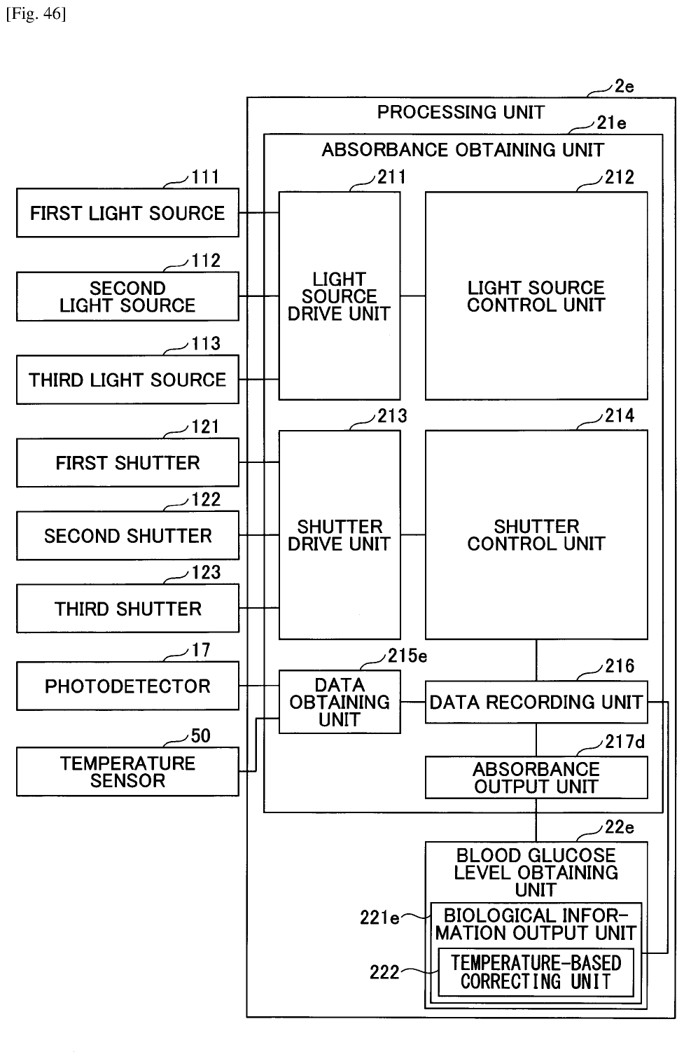

The functional configuration of a processing unit 2e provided in the blood glucose level measuring apparatus according to the present embodiment will be described with reference to FIG. 46. FIG. 46 is a block diagram illustrating an example of a functional configuration of the processing unit 2e. As depicted in FIG. 46, the processing unit 2e includes an absorbance obtaining unit 21e and a blood glucose level obtaining unit 22e. The absorbance obtaining unit 21e includes a data obtaining unit 215e, and the blood glucose level obtaining unit 22e includes a biological information output unit 221e. The biological information output unit 221e includes a temperature-based correcting unit 222.

The function of the data obtaining unit 215e is implemented by the detecting I/F 519 (see FIG. 5), and the function of the biological information output unit 221e and the temperature-based correcting unit 222 are implemented by executing of predetermined programs by the CPU 501.

The data obtaining unit 215e samples a detection signal continuously output by the photodetector 17 at a predetermined sampling cycle and outputs the detection value of the obtained light intensity to the data recording unit 216. At the same time, the temperature sensor 50 continuously outputs a detection signal at a predetermined sampling period and outputs the obtained temperature data to the data recording unit 216. The temperature sensor 50 is disposed under the tongue of a to-be-measured person corresponding to a living body S, and a thus-obtained sublingual temperature detection signal can be output to the data obtaining unit 215e. The temperature sensor 50 is an example of a temperature detector.

The biological information output unit 221e performs a predetermined calculation process on the basis of absorbance data input from the absorbance output unit 217 to obtain blood glucose level data. The temperature-based correcting unit 222 corrects the blood glucose level data on the basis of previously obtained correspondence relationships between an obtained temperature and a blood glucose level. The biological information output unit 221e is an example of “a biological information output unit that outputs biological information obtained on the basis of light intensity of probe light and a temperature of at least one of a to-be-measured object and a total reflection member.”

<Advantageous Effects of Temperature-Based Correction>

Advantageous effects of correction of blood glucose level data on the basis of temperature will now be described. First, the correlation between a temperature detected by the temperature sensor 50 (in this example, sublingual temperature) and a blood glucose level will now be described.

To investigate this correlation, an experiment was performed to measure absorbance and detect a sublingual temperature of a to-be-measured person from about 1 hour before the person's meal to about 5 hours after the meal.

A normalized multiple linear regression (MLR) model was used to obtain (calculate) a blood glucose level on the basis of a result of absorbance measurement. The normalized wavenumber of 1000 cm⁻¹ was used in the normalized MLR model. The expression for the normalized MLR model is depicted in the expression (4) below.

$$\begin{matrix}[ Math \text{.4} ] & \\ y = \frac{\begin{matrix}- 1175 \cdot x ( 1050 {cm}^{- 1} ) + 1849 \cdot x ( 1070 {cm}^{- 1} ) - \\ 859 \cdot x ( 1100 {cm}^{- 1} ) + 276\end{matrix}}{x ( 1000 {cm}^{- 1} )} & ( 4 )\end{matrix}$$

In the expression (4), y represents blood glucose level data (blood glucose level data before correction) not corrected by the temperature-based correcting unit 222, and x(k) represents absorbance data before normalization measured at the wavenumber k. Blood glucose level data can be obtained using the expression (4) above on the basis of absorbance data.

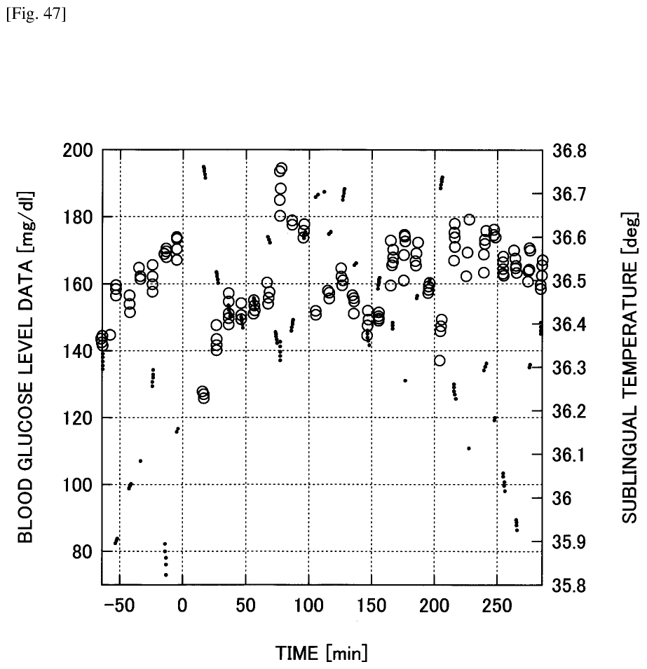

FIG. 47 is a diagram illustrating an example of a temperature detection result and a blood glucose level data obtaining result. The horizontal axis of FIG. 47 represents time, the first axis of the vertical axis (the left axis) represents a blood glucose level, and the second axis of the vertical axis (the right axis) represents a temperature detected by the temperature sensor 50. 0 minutes on the horizontal axis indicates the time at which the person ate the meal, with the minus side indicating before the meal and the plus side indicating after the meal.

The white circles in FIG. 47 represent blood glucose levels obtained, and the black dots represent detected sublingual temperatures. The white circles in FIG. 47 represent blood glucose level data before correction.

A Blood glucose level is considered to be generally low on an empty stomach before meal, and high after meal. In FIG. 47, blood glucose levels are relatively high before meal, and sublingual temperatures before meal are low. Thus, FIG. 47 suggests a correlation between sublingual temperature and a blood glucose level.

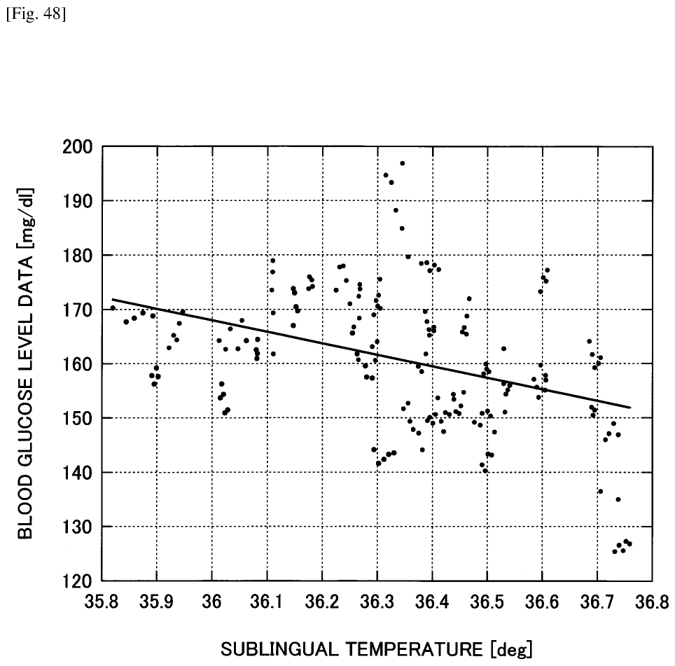

FIG. 48 depicts the results of investigating a correlation between a sublingual temperature and a blood glucose level using the data of FIG. 47. The horizontal axis of FIG. 48 depicts a sublingual temperature and the vertical axis depicts a blood glucose level. A blood glucose level should be independent of a sublingual temperature, but the negative correlation is seen in FIG. 48. The slope of the regression line in this negative correlation is −21 (mg/dl/deg). Therefore, by correcting blood glucose level data obtained on the basis of absorbance data using the slope of this regression line, more accurate blood glucose level data can be obtained. An expression for blood glucose level data correction using the slope of the regression line is as depicted in the expression (5) below.

[Math.5]

y_c=y+21×T−765 (5)

In the expression (5) above, y_c denotes corrected blood glucose level data, y denotes uncorrected blood glucose level data, and T denotes a detected sublingual temperature. The intercept “−765” is obtained from an adjustment made in such a manner that corrected blood glucose level data is almost the same regardless of a temperature.

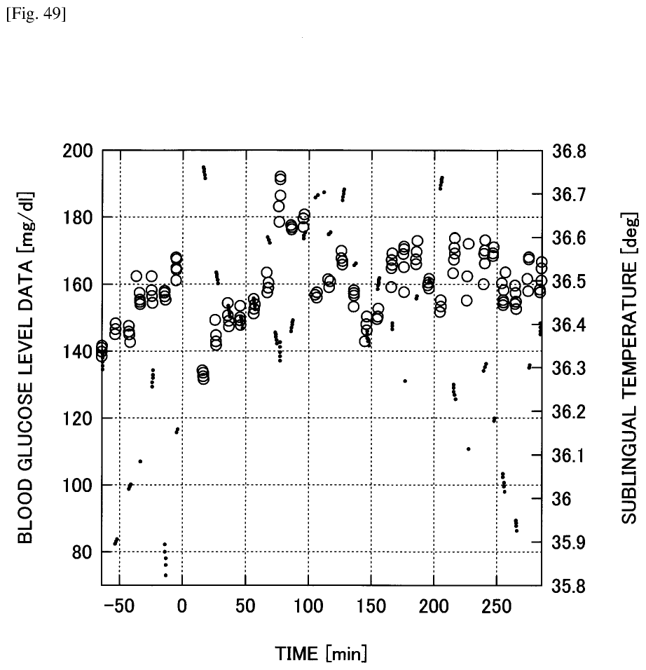

FIG. 49 is a diagram illustrating an example of a temperature detection result and a blood glucose level data obtaining result when the blood glucose level data is corrected using expression (5). Because the description as to how to view FIG. 49 is the same for FIG. 47 described above, the duplicate description will be omitted here.

Compared to FIG. 47 before correction, in FIG. 49, the blood glucose level data before meal is smaller, and even after a long time after meal, the blood glucose level data are smaller. Thus, it can be seen that the blood glucose level data is corrected in line with a tendency that a blood glucose level is lower in a fasting state before meal and also after a long period of time after meal.

With regard to the present embodiment, an example of correction on the basis of the correlation between the sublingual temperature of a living body S and blood glucose level data has been described. However, the same advantageous effects can be obtained by detecting the temperature of the ATR prism 16 instead of the body temperature of a living body S and performing correction on the basis of the correlation between a temperature of the ATR prism 16 and blood glucose level data.

<Advantageous Effects of Eleventh Embodiment>

When the ATR prism 16 is in contact with a living body S to measure a blood glucose level, blood glucose level data obtained may vary depending on the temperature of the living body S contacting the ATR prism 16 or the temperature of the ATR prism 16.

One possible reason for this phenomenon is that the ATR prism 16 itself is heated by the temperature of the contacting living body S, and the amount of mid-infrared light exiting from by the ATR prism 16 itself changes, thereby affecting the measurement. It is also possible that a contact of the ATR prism 16 causes a change in the temperature at the corresponding portion of the living body S, thereby altering the metabolism in the living body or radiation of mid-infrared light from the portion of the living body S.

In the related art, because an optical measuring unit, such as an ATR prism 16, is configured to make measurement while contacting a to-be-measured object, it may be impossible to accurately measure a blood glucose level due to an influence of the temperature of a portion of a living body S contacting the ATR prism 16 or the temperature of the ATR prism 16.

In the present embodiment, blood glucose level data is obtained on the basis of light intensity of probe light and of the temperature of at least one of a living body S and the ATR prism 16. More specifically, blood glucose level data is obtained on the basis of absorbance data obtained on the basis of light intensity of probe light, and the blood glucose level data is corrected on the basis of the temperature of the living body S detected by the temperature sensor 50.

This correction of blood glucose level data uses a correcting expression (a mathematical expression) on the basis of the correspondence relationships between a temperature and a blood glucose level previously obtained. Accordingly, a blood glucose level can be accurately measured by reducing the influence of the heat of the ATR prism 16 on the living body S and the influence of the heat of the living body S on the ATR prism 16.

With regard to the present embodiment, an example in which the temperature sensor 50 detects a sublingual temperature of a to-be-measured person corresponding to a living body S has been described, but the specific method is not limited to this method. The temperature sensor 50 may be located at any portion of a to-be-measured person's body to detect the temperature of the portion of the person's body, or the temperature sensor 50 may be located at the ATR prism 16 to detect the temperature of the ATR prism 16 or detect the temperature of the person's portion in contact with the ATR prism 16. In this regard, it is desirable to obtain the correcting expression in advance on the basis of the correspondence relationships between a temperature and a blood glucose level at each setting position of the temperature sensor 50, and use the correcting expression corresponding to the setting position of the temperature sensor 50 at a time of measurement.

When the temperature sensor 50 is disposed at the ATR prism 16, it is suitable to place a living body S in contact with the ATR prism 16 at an end of the total reflection face to prevent the temperature sensor 50 from blocking probe light and interfering with the absorbance measurement.

In addition, when body temperature data of a living body S is used, it is desirable to detect the temperature of a portion of the living body S in contact with the ATR prism 16, so that blood glucose level data can be correctly corrected. For example, when the ATR prism 16 in contact with a lip is used for measurement, it is desired to place the temperature sensor 50 at a position suitable to detect the temperature of the lip. However, a blood glucose level can be measured while contacting the ATR prism 16 also with any one of various portions other than a lip, such as an earlobe or a finger.

With respect to the present embodiment, an example of correction using a correcting expression obtained on the basis of corresponding relationships between a temperature and a blood glucose level has been described, but the specific method is not limited to this method. A table indicating the correlation relationships between a temperature and a blood glucose levels may be prepared in advance and stored in a storage device, such as the HD 504, and a corrected blood glucose level data may be obtained by referring to the table on the basis of a detected temperature at the time of measurement.

With respect to the present embodiment, an example of using a linear correcting expression has been described, but correction may be implemented using a non-linear polynomial as the correcting expression. The use of a non-linear polynomial allows for more detailed corrections.

### Twelfth Embodiment

Next, a blood glucose level measuring apparatus according to a twelfth embodiment will be described.

The twelfth embodiment is similar to the first embodiment described above with reference to FIGS. 1-14B. Therefore, mainly, the points different from the first embodiment will be described, and duplicate description may be omitted.

In the present embodiment, on the basis of relationships between first absorbance with respect to first probe light and second absorbance with respect to second probe light having a different wavelength from the first probe light among a plurality of probe lights including the first probe light and the second probe light, the second absorbance is converted to converted absorbance. Then, blood glucose level data is obtained on the basis of absorbance with respect to a plurality of probe lights including the converted absorbance. Accordingly, without obtaining data for conversion (correction) in advance, a blood glucose level is accurately measured by reducing an influence of a change in the surrounding environment of the blood glucose level measuring apparatus, the temperature of a living body, and so forth.

<Function Configuration of Processing Unit 2f>

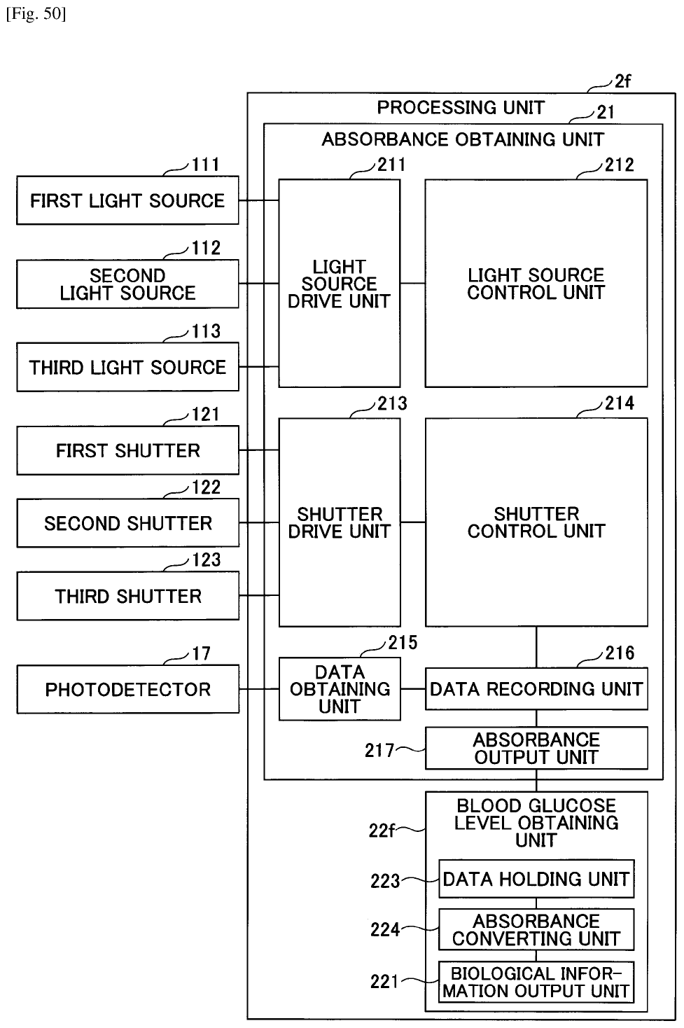

First, a functional configuration of a processing unit 2f provided in the blood glucose level measuring apparatus according to the present embodiment will be described with reference to FIG. 50. FIG. 50 is a block diagram illustrating an example of the functional configuration of the processing unit 2f. As depicted in FIG. 50, the processing unit 2f includes a blood glucose level obtaining unit 22f. The blood glucose level obtaining unit 22f includes a data holding unit 223 and an absorbance converting unit 224.

The function of the data holding unit 223 is implemented by the HD 504 (see FIG. 5), and the function of the absorbance converting unit 224 is implemented by the CPU 501 executing a predetermined program or the like.

The data holding unit 223 temporarily stores first absorbance data with respect to first probe light input from the absorbance output unit 217, second absorbance data with respect to second probe light, and third absorbance data with respect to third probe light. The data holding unit 223 can overwrite with and stores newly input first through third absorbance data after a predetermined period of time.

The absorbance converting unit 224 reads out the first through third absorbance data temporarily stored by the data holding unit 223 and, with the use of the first absorbance data as reference absorbance data, converts the second absorbance data to second converted absorbance data on the basis of the relationships between the reference absorbance data and the second absorbance data. In addition, on the basis of the relationships between the reference absorbance data and the third absorbance data, the third absorbance data is converted to third converted absorbance data. The second converted absorbance data and the third converted absorbance data are examples of converted absorbance, respectively.

It is noted that, in the present embodiment, as an example, probe light having a wavenumber of 1100 cm⁻¹ is referred to as first probe light, probe light having a wavenumber of 1050 cm⁻¹ is referred to as second probe light, and probe light having a wavenumber of 1070 cm⁻¹ is referred to as third probe light.

Thereafter, the absorbance converting unit 224 outputs the first absorbance data, the second absorbance data, and the third absorbance data to the biological information output unit 221. The biological information output unit 221 uses the first absorbance data, the second absorbance data, and the third absorbance data as input data to obtain blood glucose level data on the basis of the normalized MLR model of the above-mentioned expression (4). The normalized MLR model of the expression (4) is an example of a linear model.

<Function of Absorbance Converting Unit 224>

Next, the function of the absorbance converting unit 224 will be described. First, the correlation of each of second absorbance and third absorbance with respect to reference absorbance is described.

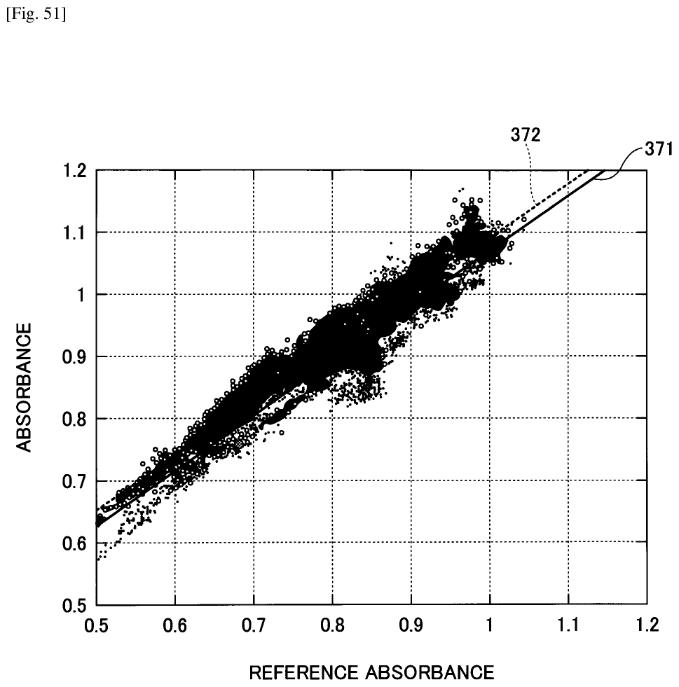

In order to investigate the correlation, first through third absorbance with respect to a lip of a to-be-measured person corresponding to a living body S were measured dozens of times from before meal through three hours after the meal. FIG. 51 depicts the correlation of each of second absorbance and third absorbance with respect to reference absorbance. The horizontal axis of FIG. 51 depicts reference absorbance. Black dots represent second absorbance, and white dots represent third absorbance.

Absorbance measured varies depending on a state of contact between a living body S and the ATR prism 16, a variation in detection sensitivity of the photodetector 17, and the like, but on average, second absorbance and third absorbance are considered to be proportional to reference absorbance. However, as depicted in FIG. 51, the regression line 371 of the second absorbance relative to the reference absorbance (the regression line of the solid line) and the regression line 372 of the third absorbance relative to the reference absorbance (the regression line of the broken line) differ in intercept values. Specifically, the intercept of the regression line of the second absorbance is 0.187 and the intercept of the regression line of the third absorbance is 0.217.

It is probable that such a difference in intercepts seems to occur due to the temperature of the surrounding environment of the blood glucose level measuring apparatus, the sensitivity difference of the photodetector 17 due to the difference in wavelength, the zero point drift, or the like. Therefore, in the present embodiment, the second and third absorbance data are converted in such a manner as to correct such a difference in intercepts.

According to the normalized MLR model, if a measurement sensitivity varies at each wavelength of probe light, blood glucose level data obtained on the basis of the absorbance varies. The measurement sensitivity corresponds to a slope of a regression line. In the example of FIG. 51, the slope of the regression line 371 is 0.883 and the slope of the regression line 372 is 0.872, indicating that the measurement sensitivity is different. Therefore, in the present embodiment, the second and third absorbance data are converted to compensate for this slope difference.

Expressions (6) and (7) below are for performing a conversion process to correct such intercept and slope differences.

[Math.6]

a1050_c=(a1050−c1050)/k1050 (6)

[Math.7]

a1070_c=(a1070−c1070)/k1070 (7)

In the expression (6), a1050_c represents second absorbance data after conversion (second converted absorbance data), a_1050 represents second absorbance data before conversion, c1050 represents the intercept of the regression line 371, and k1050 represents the slope of the regression line 371. In the expression (7), a1070_c represents third absorbance data after conversion (third converted absorbance data), a_1070 represents third absorbance data before conversion, c1070 represents the intercept of the regression line 372, and k1070 represents the slope of the regression line 372.

The second converted absorbance data and the third converted absorbance data are input to the normalized MLR model. The coefficient at each term in the normalized MLR model of expression (4) is predetermined to correspond to absorbance data after conversion.

FIG. 51 depicts an example in which absorbance was measured dozens of times from before meal through three hours after the meal in order to obtain correlation data of each of second absorbance and third absorbance with respect to reference absorbance. However, correlation data of each of second absorbance and third absorbance with respect to reference absorbance can be obtained even from the smaller number of times of absorbance measurement.

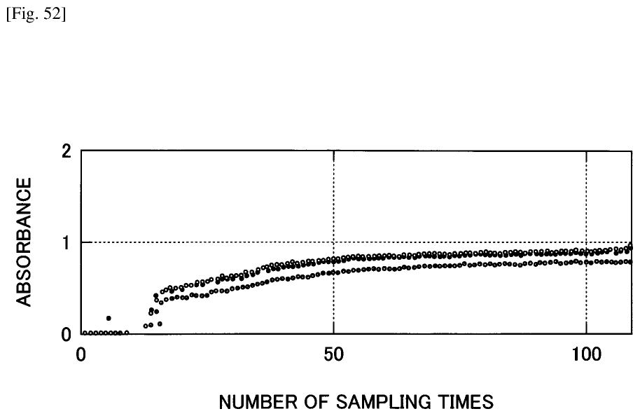

FIG. 52 depicts reference absorbance, second absorbance and third absorbance at a single absorbance measurement. Absorbance data is sampled multiple times at a single absorbance measurement. The horizontal axis of FIG. 52 indicates the number of sampling times and the vertical axis indicates absorbance. In the example depicted in FIG. 52, the number of sampling times in one absorbance measurement is 120. The graph in FIG. 52 depicts results of measurements of reference absorbance, second absorbance, and third absorbance in a mixed manner.

At the approximately 15th sampling, the ATR prism 16 became in contact with a lip and then absorbance increased. However, the absorbance did not become constant after the contact, but increased gradually. This is because of a change in the contact state between the ATR prism 16 and the lip, or a change in the temperature of the ATR prism 16 or the lip due to the contact of the ATR prism 16 with the lip. The time required for the single measurement is approximately 1 minute.

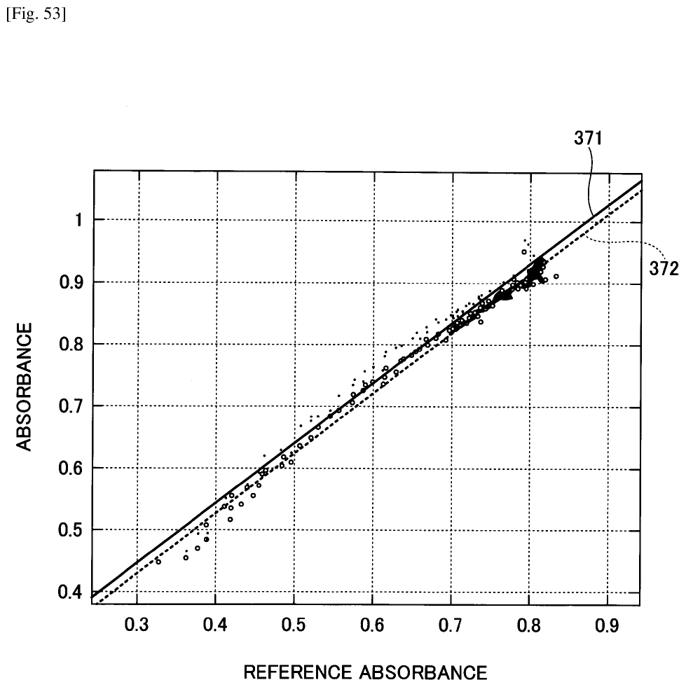

The correlation of each of second and third absorbance with respect to reference absorbance obtained using the measurement results in FIG. 52 are depicted in FIG. 53. Because the description as to how to view FIG. 53 is the same for FIG. 51 described above, the duplicate description will be omitted.

As depicted in FIG. 53, the regression line 371 of second absorbance and the regression line 372 of third absorbance can be obtained even through a single absorbance measurement. Then, the slope and intercept of the regression line 371 can be used to obtain second converted absorbance data, and the slope and intercept of the regression line 372 can be used to obtain third converted absorbance data .

In blood glucose level measurement according to the present embodiment, the absorbance converting unit 224 reads the first through third absorbance data temporarily stored by the data holding unit 223. Then, the slope and intercept of the regression line 371 of the second absorbance data, obtained by using the first absorbance data as the reference absorbance data, are then used to obtain the second converted absorbance data. In addition, the third conversion absorbance data is obtained by using the slope and intercept of the regression line 372 of the third absorbance data, obtained by using the first absorbance data as the reference absorbance data.

Thus, the absorbance data can be converted in a manner of removing the influence of the temperature of the ambient environment of the blood glucose level measuring apparatus, the sensitivity difference of the photodetector 17 due to the difference in the wavelength, the zero point drift, and the like, without the need of obtaining data for conversion (correction) in advance.

<Advantageous Effect According to Twelfth Embodiment>

As described above, in the present embodiment, among a plurality of probe lights including first probe light and second probe light having a different wavelength from the first probe light, first absorbance data with respect to the first probe light and second absorbance data with respect to the second probe light, as examples of first absorbance and second absorbance, are used to determine a regression line 371. Then, the slope and intercept of the regression line 371 are used to convert the second absorbance data into second converted absorbance data, and a blood glucose level is measured on the basis of absorbance data with respect to a plural sets of probe data including the second converted absorbance data.

Because previously obtained data for conversion (correction) is not used, second absorbance data can be converted to second converted absorbance data in such a manner that, even though the measurement conditions vary by the minute due to a variation in the ambient environment or a variation in the temperature of a living body, the variation in the ambient environment of the blood glucose level measuring apparatus or the variation in the temperature of the living body can be removed depending on the variation. This can reduce the influence of the variations in the surrounding environment and temperature of the living body, to enable implementing accurate measurement of a blood glucose level.

With regard to the present embodiment, an example of the conversion process using the slope and intercept of the regression line is described, but the specific method is not limited to the above-described method. Because the photodetector 17 may have non-linear sensitivity characteristics, in such a case, the conversion process may be performed using at least one of the coefficients at the respective terms in a regression polynomial of a quadratic or cubic expression, for example. Accordingly, even when the photodetector 17 has non-linear sensitivity characteristics, an influence of a surrounding environment of the blood glucose level measuring apparatus and a temperature variation of a living body can be further reduced finely, and a blood glucose level can be accurately measured.

Further, in the present embodiment, the blood glucose level obtaining unit 22f includes the data holding unit 223. However, instead, the function of the data holding unit 223 may be provided in the data recording unit 216, an external memory device, or the like.

In the present embodiment, the conversion process is performed using both a slope and an intercept. However, the conversion process may be performed using at least one of a slope and an intercept.

Although the measuring apparatuses, the biological information measuring apparatuses, and the absorbance measuring apparatuses have been described above with regard to the embodiments, variations and modifications can be made to the embodiments.

With regard to the embodiments, an example in which the functions of the absorbance obtaining unit 21, the blood glucose level obtaining unit 22, the drive control unit 23, and so forth are implemented by the processing unit has been described, but, instead, these functions may be implemented also by separate processing units, or the functions of the absorbance obtaining unit 21 and the blood glucose level obtaining unit 22 may be distributed among a plurality of processing units. In addition, the function of the processing unit and the function of the storage device such as the data recording unit 216 can be implemented by an external apparatus such as a cloud server is implemented.

With regard to the embodiments, the example where the first light source 111, the second light source 112, and the third light source 113 are used as the plurality of light sources, each of which emits light of different wavelengths in the mid-infrared region, but, instead, a single light source may be used to emit light of multiple wavelengths.

Also, although the examples using the quantum cascade lasers have been described as the light sources, the light sources are not limited to quantum cascade lasers. Light sources other than lasers such as infrared lamps, light emitting diodes (LED), super luminescent diodes (SLD) may be used instead. In such a case, it is desirable to use a wavelength filter for obtaining only a desired wavelength to cause probe light to be incident on the total reflection member, such as the ATR prism 16, through the filter. Alternatively, the photodetector 17 may desirably receive probe light through a wavelength filter.

With regard to the embodiments described above, the examples of measuring blood glucose levels as biological information have been described. However, as long as it is possible to measure using the ATR method, any other biological information can be measured with the use of any one of the embodiments.

In addition, an optical element, such as a beam splitter, for branching a portion of probe light after the probe light is emitted by the light source or exits from the hollow optical fiber, and a detection element for detecting the probe light intensity of the thus branched portion may be provided to implement feedback control of the driving voltage or the driving current of the light source so as to reduce a variation in probe light intensity. This reduces a variation in output of the light source and allows for more accurate measurement of biological information.

Further, an example of a total reflection member including the ATR prism 16 has been described, but is not limited to this example. A total reflection member may be provided using parallel plates, an optical fiber, or the like, provided that total reflection can be caused to occur and penetration of a field upon total reflection can be caused to occur.

Further, although the examples of applying the seventh through twelfth embodiments to the configuration of the blood glucose level measuring apparatus 100 according to the first embodiment have been described, examples are not limited to these examples. Each of the seventh through tenth embodiments can be applied also when a blood glucose level measuring apparatus includes one light source and emits first through third probe lights of different wavelengths from the one light source. In that case, the blood glucose level measuring apparatus need not include the first shutter 121, the second shutter 122, the third shutter 123, the first half mirror 131, and the second half mirror 132, as incidences of first through third probe lights on the ATR prism 16 need not be switched.

In addition, each of the seventh through eleventh embodiments can be applied to a blood glucose level measuring apparatus that includes one light source and emits one wavelength of probe light from the one light source.

In addition, each of the seventh through twelfth embodiments can be applied to absorbance measurement and biological information measurement where light intensities of first through third probe lights are not corrected using a detection value of the photodetector 17 during a non-incidence period .

In addition, a blood glucose level measuring apparatus may be configured by combining a plural embodiments from among the seventh through twelfth embodiments.

Absorbance measuring methods are also included in embodiments of the present invention. For example, an absorbance measuring method includes: emitting a plurality of probe lights of different wavelengths in a specific wavelength region; causing total reflection of incident probe light by a total reflection member in a state of being in contact with a to-be-measured object; controlling incidence of the probe light to the total reflection member in such a manner that there is a period in which all of the plurality of probe lights are not incident on the total reflection member; detecting by a light intensity detector the probe light exiting from the total reflection member; and outputting absorbance obtained on the basis of the detection value of the light intensity detector when the probe light is incident on the total reflection member and the detection value of the light intensity detector when all of the plurality of probe lights are not incident on the total reflection member. By such an absorbance measuring method, the same advantageous effects as the advantageous effects of the absorbance measuring apparatus according to the seventh embodiment can be obtained.

As yet another embodiment, an absorbance measuring method includes: emitting probe light in a specific wavelength region; causing total reflection of the incident probe light by a total reflection member in a state of being in contact with a to-be-measured object; guiding the probe light to the total reflection member by a light guide; driving the light guide; controlling driving of the light guide; detecting the light intensity of the probe light exiting from the total reflection member; and outputting absorbance with respect to the probe light obtained on the basis of the detected light intensity. Such an absorbance measuring method can obtain the same advantageous effects as the advantageous effects of the absorbance measuring apparatus according to the eighth embodiment.

As a further another embodiment, a biological information measuring method includes: emitting probe light in a specific wavelength region; causing total reflection of the incident probe light by a total reflection member in a state of being in contact with a to-be-measured object; detecting light intensity of the probe light exiting from the total reflection member; and outputting biological information obtained on the basis of the detected light intensity and a temperature of at least one of the to-be-measured object and the total reflection member. Such a biological information measuring method can obtain the same advantageous effects as the advantageous effects of the biological information measuring apparatus according to the eleventh embodiment.

As a still another embodiment, a biological information measuring method includes: emitting a plurality of probe lights including first probe light and second probe light having a different wavelength from the first probe light; detecting light intensity of the probe light after the probe light is partially absorbed by a to-be-measured object; obtaining absorbance with respect to the probe light on the basis of the detected light intensity; converting, on the basis of a relationship between first absorbance with respect to the first probe light and second absorbance with respect to the second probe light, the second absorbance to converted absorbance; and outputting biological information obtained on the basis of absorbance with respect to the plurality of probe lights including the converted absorbance. Such a biological information measuring method can obtain the same advantageous effects as the advantageous effects of the biological information measuring apparatus according to the twelfth embodiment.

The functions of each of the embodiments described above may also be implemented by one or more processing circuits. The “processing circuit” used herein includes a processor programmed to perform each function by software, such as a processor implemented by electronic circuits, an application specific integrated circuit (ASIC), a digital signal processor (DSP), a field programmable gate array (FPGA), or a conventional circuit module designed to perform each function as described above.

The present disclosures non-exhaustively include the subject matter set out in the following clauses:

Clause 1. An optical member including:

a total reflection member that includes a total reflection face configured to, in contact with an object, cause total reflection of probe light that is incident; and

a hollow section inside the total reflection member.

Clause 2. The optical member according to clause 1,

wherein

the total reflection member is made of a silicon material.

Clause 3. The optical member according to clause 1 or 2,

wherein

an inclined face is provided at a portion of the hollow section facing the total reflection face and is inclined from the total reflection face at an angle equal to an angle of incidence of the probe light on the total reflection face.

Clause 4. The optical member according to clause 3,

wherein

the inclined face is inclined from the total reflection face at an angle greater than or equal to a critical angle.

Clause 5. The optical member according to clause 3 or 4,

wherein

the inclined face is provided with antireflective coating that prevents reflection of the probe light.

Clause 6. The optical member according to any one of clauses 3-5,

wherein

the probe light has a p-polarized state and is incident on the inclined face from the hollow section at an angle corresponding to a Brewster angle.

Clause 7. The optical member according to any one of clauses 1 to 6,

wherein

the total reflection member includes a plurality of plate-like members, and

the hollow section is a gap between the plurality of plate-like members.

Clause 8. The optical member according to any one of clauses 1-6,

wherein

the total reflection member includes a first plate-like member and a second plate-like member opposite to the first plate-like member, and

the hollow section is a gap between the first plate-like member and the second plate-like member.

Clause 9. The optical member according to any one of clauses 1-6,

wherein

the total reflection member includes a first plate-like member including the total reflection face, and a reflecting member opposite to the first plate-like member and including a reflecting face, and

the hollow section is a gap between the first plate-like member and the reflecting member.

Clause 10. A biological information measuring apparatus, including:

the optical member according to any one of clauses 1-9;

a light source configured to emit the probe light;

a light intensity detector configured to detect light intensity of the probe light exiting from the optical member; and

a biological information output unit configured to output biological information obtained on the basis of the light intensity,

wherein

the object is a to-be-measured object.

Clause 11. The biological information measuring apparatus according to clause 10,

wherein

the biological information is blood glucose level information.

Clause 12. The biological information measuring apparatus according to clause 11,

wherein
the probe light has at least any one of wavenumbers 1050 cm⁻¹, 1070 cm⁻¹, and 1100 cm⁻¹.

Clause 13. An absorbance measuring apparatus, including:

a light source configured to emit probe light in a specific wavelength region;

a total reflection member configured to, in contact with a to-be-measured object, cause total reflection of the incident probe light;

a pressure detector configured to detect a pressure of the to-be-measured object to the total reflection member;

a light intensity detector configured to detect light intensity of the probe light exiting from the total reflection member; and

an absorbance output unit configured to output absorbance with respect to the probe light on the basis of the light intensity and the pressure.

Clause 14. The absorbance measuring apparatus according to clause 13, further including

an indicating unit configured to indicate the pressure.

Clause 15. The absorbance measuring apparatus according to clause 13 or 14,

wherein

the absorbance output unit is configured to output absorbance corrected on the basis of the pressure.

Clause 16. The absorbance measuring apparatus according to any one of clauses 13-15,

wherein

the pressure detector is configured to detect the pressure at several points of the to-be-measured object.

Clause 17. The absorbance measuring apparatus according to clause 15 or 16,

wherein

the to-be-measured object is a living body, and

the pressure detector is configured to be in contact with a lip of the living body to detect the pressure.

Clause 18. The absorbance measuring apparatus according to any one of clauses 15-17,

wherein

the pressure detector is configured to detect pressures of upper and lower lips of the living body.

Clause 19. The absorbance measuring apparatus according to any one of clauses 15-18,

wherein

the pressure detector is provided at the total reflection member.

Clause 20. The absorbance measuring apparatus of clause 19,

wherein

the pressure detector is at a predetermined portion of a total reflection face of the total reflection member.

Clause 21. The absorbance measuring apparatus according to clause 19 or 20,

wherein

the pressure detector is at an end of a total reflection face of the total reflection member.

Clause 22. The absorbance measuring apparatus according to clause 19 or 20,

wherein

a plurality of pressure detectors are at a total reflection face of the total reflection member.

Clause 23. The absorbance measuring apparatus according to any one of clauses 13-22, further including

a total reflection support configured to support the total reflection member,

wherein

the pressure detector is at, at least one of the total reflection member and the total reflection support.

Clause 24. The absorbance measuring apparatus according to clause 23,

wherein

a condition “Tₛᵤₚ<tₐₜᵣ” is satisfied where tₛᵤₚ denotes a thickness of the total reflection support and tₐₜᵣ denotes a thickness of the total reflection member.

Clause 25. The absorbance measuring apparatus according to clause 23 or 24,

wherein

a condition “(tₐₜᵣ−tₛᵤₚ)/2≤tₛₑₙ” is satisfied where tₛᵤₚ denotes a thickness of the total reflection support, tₐₜᵣ denotes a thickness of the total reflection member, and tₛₑₙ denotes a thickness of the pressure detector.

Clause 26. The absorbance measuring apparatus according to any one of clauses 23-25,

wherein

a condition “0≤tₛₑₙ−(t^{atr}−tₛᵤₚ)/2<1 (mm)” is satisfied where tₛᵤₚ denotes a thickness of the total reflection support, tₐₜᵣ denotes a thickness of the total reflection member, and tₛₑₙ denotes a thickness of the pressure detector.

Clause 27. The absorbance measuring apparatus according to any one of clauses 13-26,

wherein

the pressure detector includes at least one of a capacitive pressure sensor, a strain gauge pressure sensor, a pressure-sensitive resistance type sensor, and a micro mechanical electro system (MEMS) pressure sensor.

Clause 28. A biological information measuring apparatus, including:

the absorbance measuring apparatus according to any one of clauses 13-27; and

a biological information output unit configured to output biological information obtained on the basis of the absorbance.

Clause 29. The biological information measuring apparatus according to clause 28,

wherein

the biological information is blood glucose level information.

Clause 30. The biological information measuring apparatus according to clause 29,

wherein

the probe light includes at least any one of wavenumbers 1050 cm¹, 1070 cm⁻¹, and 1100 cm⁻¹.

Clause 31. An absorbance measuring apparatus, including:

a light source configured to emit probe light in a specific wavelength region;

a total reflection member including an incidence face on which the probe light emitted from the light source is incident; a total reflection face from which, in a state of the total reflection face being in contact with a to-be-measured object, the probe light undergoes total reflection; and an outgoing face from which the probe light having undergone total reflection by the total reflection face exits;

a light intensity detector configured to detect light intensity of the probe light exiting from the outgoing face; and

an absorbance output unit configured to output absorbance of the probe light obtained on the basis of the light intensity,

wherein

the total reflection member includes an area defining section configured to define a measurement sensitivity area for measuring the absorbance in the total reflection face.

Clause 32. The absorbance measuring apparatus according to clause 31,

wherein

the area defining section is configured to define the measurement sensitivity area in such a manner that the probe light undergoes total reflection at the measurement sensitivity area.

Clause 33. The absorbance measuring apparatus according to clause 31 or 32.

wherein

the area defining section is configured to define the measurement sensitivity area in such a manner that the probe light undergoes total reflection at an area other than an end of the total reflection face.

Clause 34. The absorbance measuring apparatus according to any one of clauses 31-33,
wherein
the area defining section is configured to define the measurement sensitivity area by providing a reflective film configured to reflect the probe light at an area other than the measurement sensitivity area.

Clause 35. The absorbance measuring apparatus according to clause 34,

wherein

the reflective film is made of at least one of a gold material and a silver material.

Clause 36. The absorbance measuring apparatus according to any one of clauses 31-35,

wherein

the incidence face includes a diffusing surface.

Clause 37. The absorbance measuring apparatus according to any one of clauses 31-36,

wherein

the incidence face has a curvature.

Clause 38. The absorbance measuring apparatus according to any one of clauses 31-37, further including:

a light guide configured to guide the probe light to the total reflection member;

a drive unit configured to drive the light guide; and

a drive control unit configured to control the drive unit.

Clause 39. The absorbance measuring apparatus according to clause 38,

wherein

the drive unit changes at least one of a position and an angle of the light guide.

Clause 40. The absorbance measuring apparatus according to clause 38 or 39, further including

a light guide support supporting the total reflection member and the light guide.

Clause 41. The absorbance measuring apparatus according to any one of clauses 31-40, further including

a pressure detector configured to detect a pressure of the to-be-measured object on the total reflection member,

wherein

the absorbance output unit is configured to output the absorbance obtained on the basis of the light intensity of the probe light and the pressure.

Clause 42. The absorbance measuring apparatus according to any one of clauses 31-41,

wherein

the probe light is changed in light intensity at a predetermined cycle.

Clause 43. The absorbance measuring apparatus according to clause 42,

wherein

the probe light is changed in light intensity at three or more levels.

Clause 44. A biological information measuring apparatus, including:

the absorbance measuring apparatus according to any one of clauses 31-43; and

a biological information output unit configured to output biological information obtained on the basis of the absorbance.

Clause 45. The biological information measuring apparatus according to clause 44,

wherein

the biological information is blood glucose level information.

Clause 46. The biological information measuring apparatus according to clause 45,

wherein

the probe light includes at least any one of wavenumbers 1050 cm⁻¹, 1070 cm⁻¹, and 1100 cm⁻¹.

Although the measuring apparatuses and biological information measuring apparatuses have been described with reference to the embodiments, embodiments of the present invention are not limited to the above-described embodiments, and variations can be made within the scope of the present invention.

The present application is based on and claims priority to Japanese patent application No. 2019-195633 filed on Oct. 28, 2019, Japanese patent application No. 2019-195636 filed on Oct. 28, 2019, Japanese patent application No. 2019-201307 filed on Nov. 6, 2019, and Japanese patent application No. 2019-201786 filed on Nov. 6, 2019. The entire contents of Japanese patent application No. 2019-195633, Japanese patent application No. 2019-195636, Japanese patent application No. 2019-201307, and Japanese patent application No. 2019-201786 are hereby incorporated herein by reference.

### REFERENCE SIGNS LIST

1, 1a, 1b, 1c Measuring units

100, 100a, 100b, 100c Blood glucose level measuring apparatus (example of biological information measuring apparatus)

101, 101a, 101b, 101c Absorbance measuring apparatus

110 QCL (an example of a light source)

111 First light source (an example of a light source)

112 Second light source (an example of a light source)

113 Third light source (an example of a light source)

121 First shutter

122 Second shutter

123 Third shutter

131 First half mirror

132 Second half mirror

14 Coupling lens

151 First hollow optical fiber (an example of a light guide)

152 Second hollow optical fiber

153 Light guide support

154 Outgoing support

16 ATR prism (an example of a total reflection member)

161 Incidence face

162 First total reflection face

162m, 163m reflective films (an example of an area defining section)

162k, 163k fields

163 Second total reflection face

164 Outgoing face

17 Photodetector (an example of a light intensity detector)

181 Light source support

182 Photodetector support

1820 Piezoelectric drive unit (an example of a drive unit)

1821 Motor (an example of a drive unit)

1822 MEMS mirror (an example of a drive unit)

183 Piezoelectric drive unit (an example of a drive unit)

191 Deflecting mirror (an example of deflecting unit)

192 First condenser lens

193 Second condenser lens (an example of a condensing unit)

2, 2a, 2b, 2c, 2d, 2e, 2f Processing units

21, 21d, 21e Absorbance obtaining units

211 Light source drive unit

212 Light source control unit

213 Shutter drive unit

214 Shutter control unit (an example of an incident control unit)

215, 215d, 215e Data obtaining units

216 Data recording unit

217, 217d Absorbance output units

22 Blood glucose level obtaining unit

221, 221e Biological information output units (an example of an output unit)

218 Indicating unit

219 Pressure-based correcting unit

222 Temperature-based correcting unit

223 Data holding unit

224 Absorbance converting unit

23 Drive control unit

26 Optical member

260 Total reflection member

260a First optical block (one example of first plate-like member)

260b Second Optical Block (Example of second plate-like member)

261 Incidence face

262 First total reflection face

263 Second total reflection face

264 Outgoing face

270 Hollow section

271-274 Inclined faces

281-283 Protrusions

30 Pressure sensor (an example of a pressure detector)

31 First support

311 Box-shaped member

312 Back plate

313 Tap hole (one example of a coupling unit)

314 Knock pin (an example of a coupling unit)

315 Knock hole (an example of a coupling unit)

316 Knock pins (an example of a plurality of coupling units)

317 Convex or protrusion with latches (an example of coupling unit)

32 Second support

321 Through hole (an example of a to-be-coupled unit)

322, 324 Knock holes (examples of a to-be-coupled unit)

323 Knock pin (an example of a to-be-coupled unit)

325 Concave or recess with latches (an example of a to-be-coupled unit)

326 Open section

33 Total reflection support

460b Mirror (an example of a reflecting member)

50 Temperature sensor (an example of a temperature detector)

501 CPU

506 Display

519 Detecting I/F

560a First optical block (one example of a plurality of plate-like members)

560b Second Optical Block (one example of a plurality of plate-like members)

560c Third optical block (one example of a plurality of plate-like members)

85 Cycle (an example of one cycle)

86 Period (one example of the first incidence period)

87 Period (one example of a second incidence period)

84,88 Non-incidence periods

P Probe light

Pr contact pressure (example of pressure)

S Living body (an example of a to-be-measured object)

y Blood glucose level data before correction

y_c Blood glucose level data after correction

T Sublingual temperature

θ₀ Angle of incidence

θ₁-θ₄ Inclined angles

θ_{C} Critical angle

φ Brewster angle

### CITATION LIST

### Patent Literature

[PTL 1] Japanese Patent No. 5376439

[PTL 2] Japanese Unexamined Patent Application Publication No. H06-281568

[PTL 3] Japanese Patent No. 4047903

1. A measuring apparatus comprising:
a light source to emit probe light;
a total reflection structure in contact with a to-be-measured object and to cause total reflection of the probe light that is incident;
a light intensity detector to detect light intensity of the probe light exiting from the total reflection structure;
a first support supporting the light source and the light intensity detector; and
a second support provided to the first support, detachable from the first support, and supporting the total reflection structure.

2. The measuring apparatus according to claim wherein:
the second support supports a face of the total reflection structure orthogonal to a total reflection face.

3. The measuring apparatus according to claim 1, wherein:
the first support includes a coupler,
the second support includes a structure to be coupled with the coupler, and
the structure to be coupled has a predetermined positional relationship with the total reflection structure supported by the second support.

4. The measuring apparatus according to claim 1, wherein:
the first support has a plurality of couplers at a face that is in contact with the second support,
the second support has a plurality of structures to be coupled at a face that is in contact with the first support, the structures to be coupled being coupled with the plurality of couplers, respectively, and
at least two of the plurality of couplers are positioned asymmetrically relative to a center of the face of the first support.

5. The measuring apparatus according to claim 1, further comprising:
a light guide to guide the probe light to the total reflection structure,
wherein the first support further supports the light guide.

6. The measuring apparatus according to claim 1, wherein:
the second support includes an open section below the total reflection structure.

7. A biological information measuring apparatus, comprising:
the measuring apparatus according to claim 1,
the measuring apparatus further comprising output circuitry configured to output a measurement value obtained using the light intensity which has been detected;
wherein the output circuitry of the measuring apparatus is further configured to output biological information obtained on the basis of the light intensity.

8. The biological information measuring apparatus according to claim 7, wherein:
the to-be-measured object is a living body, and
the second support supports a face of the total reflection structure, the face being opposite to a face which the living body faces, from among two faces of the total reflection structure orthogonal to a total reflection face of the total reflection structure.

9. The biological information measuring apparatus according to claim 7, wherein:
the to-be-measured object is a lip of a living body, and
the second support supports, from among two faces of the total reflection structure orthogonal to a total reflection face of the total reflection structure, a face opposite to a face which the living body faces when the lip is in contact with the total reflection face.

10. The biological information measuring apparatus according to claim 7, wherein:
the biological information is blood glucose level information.

11. The biological information measuring apparatus according to claim 10, wherein:
the probe light has at least any one of wavenumbers 1050 cm⁻¹, 1070 cm⁻¹, and 1100 cm⁻¹.
